SEQUENCE LISTING (44 sequences)

SEQ ID NO: 1 - protein, 3460 aa, Homo sapiens [sequence omitted]

SEQ ID NO: 2 - protein, 3461 aa, Mus musculus
MERGCWAPRALVLAVLLLLATLRARAATGYYPRFSPFFFLCTHHGELEGDGEQGEVLISLHIAGNPTYYVPGQEYHVTISTSTFFDGLLVTGLYTSTSIQSSQSIGGSSAFGFGIMSDHQFGNQFMCSVVASHVSHLPTTNLSFVWIAPPAGTGCVNFMATATHRGQVIFKDALAQQLCEQGAPTEATAYSHLAEIHSDSVILRDDFDSYQQLELNPNIWVECSNCEMGEQCGTIMHGNAVTFCEPYGPRELTTTCLNTTTASVLQFSIGSGSCRFSYSDPSITVSYAKNNTADWIQLEKIRAPSNVSTVIHILYLPEEAKGESVQFQWKQDSLRVGEVYEACWALDNILVINSAHREVVLEDNLDPVDTGNWLFFPGATVKHSCQSDGNSIYFHGNEGSEFNFATTRDVDLSTEDIQEQWSEEFESQPTGWDILGAVVGADCGTVESGLSLVFLKDGERKLCTPYMDTTGYGNLRFYFVMGGICDPGVSHENDIILYAKIEGRKEHIALDTLTYSSYKVPSLVSVVINPELQTPATKFCLRQKSHQGYNRNVWAVDFFHVLPVLPSTMSHMIQFSINLGCGTHQPGNSVSLEFSTNHGRSWSLLHTECLPEICAGPHLPHSTVYSSENYSGWNRITIPLPNAALTRDTRIRWRQTGPILGNMWAIDNVYIGPSCLKFCSGRGQCTRHGCKCDPGFSGPACEMASQTFPMFISESFGSARLSSYHNFYSIRGAEVSFGCGVLASGKALVFNKDGRRQLITSFLDSSQSRFLQFTLRLGSKSVLSTCRAPDQPGEGVLLHYSYDNGITWKLLEHYSYVNYHEPRIISVELPDDARQFGIQFRWWQPYHSSQGEDVWAIDEIVMTSVLFNSISLDFTNLVEVTQSLGFYLGNVQPYCGHDWTLCFTGDSKLASSMRYVETQSMQIGASYMIQFSLVMGCGQKYTPHMDNQVKLEYSANHGLTWHLVQEECLPSMPSCQEFTSASIYHASEFTQWRRVTVVLPQKTWSGATRFRWSQSYYTAQDEWALDNIYIGQQCPNMCSGHGSCDHGVCRCDQGYQGTECHPEAALPSTIMSDFENPSSWESDWQEVIGGEVVKPEQGCGVVSSGSSLYFSKAGKRQLVSWDLDTSWVDFVQFYIQIGGESAACNKPDSREEGILLQYSNNGGIQWHLLAEMYFSDFSKPRFVYLELPAAGKTPCTRFRWWKPVFSGEDYDQWAVDDIIILSEKQKQVIPVVNPTLPQNFYEKPAFDYPMNQMSVWLMLANEGMAKNDSFCATTPSAMVFGKSDGDRFAVTRDLTLKPGYVLQFKLNIGCTSQFSSTAPVLLQYSHDAGMSWFLLKEGCFPASAAKGCEGNSRELSEPTVYYTGDFEEWTRITIAIPRSLASSKTRFRWIQESSSQKNVPPFGLDGVYISEPCPSYCSGHGDCISGVCFCDLGYTAAQGTCVSNTPNHSEMFDRFEGKLSPLWYKITGGQVGTGCGTLNDGRSLYFNGLGKREARTVPLDTRNISLVQFYIQIGSKTSGITYITPRARYEGLVVQYSNDNGILWHLLRELDFMSFLEPQIISIDLPREAKTPATAFRWWQPQHGKHSAQWALGDVLIGVNDSSQTGFQDKLDGSIDLQANWYRIQGGQVDIDCLSMDTALIFTENIGNPRYAETWDFHVSESSFLQWEMNMGCSKPFSGAHGIQLQYSLNNGKDWQLVTEECVPPTIGCVHYTESSTYTSERFQNWRRVTVYLPLATNSPRTRFRWIQTNYTVGADSWAIDNVILASGCPWMCSGRGICDSGRCVCDRGFGGPFCVPVVPLPSILKDDFNGNLHPDLWPEVYGAERGNLNGETIKSGTCLIFKGEGLRMLISRDLDCTNTMYVQFSLRFIAKGTPERSHSILLQFSVSGGVTWHLMDEFYFPQTTSILFINVPLPYGAQTNATRFRLWQPYNNGKKEEIWIIDDFIIDGNNLNNPVLLLDTFDFGPREDNWFFYPGGNIGLYCPYSSKGAPEEDSAMVFVSNEVGEHSITTRDLSVNENTIIQFEINVGCSTDSSSADPVRLEFSRDFGATWHLLLPLCYHSSSLVSSLCSTEHHPSSTYYAGTTQGWRREVVHFGKLHLCGSVRFRWYQGFYPAGSQPVTWAIDNVYIGPQCEEMCYGHGSCINGTKCICDPGYSGPTCKISTKNPDFLKDDFEGQLESDRFLLMSGGKPSRKCGILSSGNNLFFNEDGLRMLVTRDLDLSHARFVQFFMRLGCGKGVPDPRSQPVLLQYSLNGGLSWSLLQEFLFSNSSNVGRYIALEMPLKARSGSTRLRWWQPSENGHFYSPWVIDQILIGGNISGNTVLEDDFSTLDSRKWLLHPGGTKMPVCGSTGDALVFIEKASTRYVVTTDIAVNEDSFLQIDFAASCSVTDSCYAIELEYSVDLGLSWHPLVRDCLPTNVECSRYHLQRILVSDTFNKWTRITLPLPSYTRSQATRFRWHQPAPFDKQQTWAIDNVYIGDGCLDMCSGHGRCVQGSCVCDEQWGGLYCDEPETSLPTQLKDNFNRAPSNQNWLTVSGGKLSTVCGAVASGLALHFSGGCSRLLVTVDLNLTNAEFIQFYFMYGCLITPSNRNQGVLLEYSVNGGITWNLLMEIFYDQYSKPGFVNILLPPDAKEIATRFRWWQPRHDGLDQNDWAIDNVLISGSADQRTVMLDTFSSAPVPQHERSPADAGPVGRIAFEMFLEDKTSVNENWLFHDDCTVERFCDSPDGVMLCGSHDGREVYAVTHDLTPTENWIMQFKISVGCKVPEKIAQNQIHVQFSTDFGVSWSYLVPQCLPADPKCSGSVSQPSVFFPTEGWKRITYPLPESLTGNPVRFRFYQKYSDVQWAIDNFYLGPGCLDNCGGHGDCLKEQCICDPGYSGPNCYLTHSLKTFLKERFDSEEIKPDLWMSLEGGSTCTECGVLAENTALYFGGSTVRQAITQDLDLRGAKFLQYWGRIGSENNMTSCHRPVCRKEGVLLDFSTDGGITWTLLHEMDFQKYISVRHDYILLPEGALTNTTRLRWWQPFVISNGLVVSGVERAQWALDNILIGGAEINPSQLVDTFDDEGSSHEENWSFYPNAVRTAGFCGNPSFHLYWPNKKKDKTHNALSSRELIIQPGYMMQFKIVVGCEATSCGDLHSVMLEYTKDARSDSWQLVQTQCLPSSSNSIGCSPFQFHEATIYNAVNSSSWKRITIQLPDHVSSSATQFRWIQKGEETEKQSWAIDHVYIGEACPKLCSGHGYCTTGAVCICDESFQGDDCSVFSHELPSYIKDNFESARVTEANWETIQGGVIGSGCGQLAPYAHGDSLYFNGCQIRQAATKPLDLTRASKIMFVLQIGSPAQTDSCNSDLSGPHTVDKAVLLQYSVNNGITWHVIAQHQPKDFTQAQRVSYNVPLEARMKGVLLRWWQPRHNGTGHDQWALDHVEVVLVSTRKQNYMMNFSRQHGLRHFYNRRRRSLRRYP

SEQ ID NO: 3 - DNA, 20 bp, Artificial Sequence
  Synthetic- Bcl2l11 F
gagatacggattgcacagga

SEQ ID NO: 4 - DNA, 20 bp, Artificial Sequence
  Synthetic- Bvl2l11 R
atttgagggtggtcttcagc

SEQ ID NO: 5 - DNA, 21 bp, Artificial Sequence
  Synthetic- P21 F
gaaagaagcggaagatcctcc

SEQ ID NO: 6 - DNA, 20 bp, Artificial Sequence
  Synthetic- P21 R
gggcctcagggattgtttgg

SEQ ID NO: 7 - DNA, 20 bp, Artificial Sequence
  Synthetic- Pdcd4 F
gaaattggatttccgcatct

SEQ ID NO: 8 - DNA, 19 bp, Artificial Sequence
  Synthetic- Pdcd4 R
taaccgcttcacttccatt

SEQ ID NO: 9 - DNA, 20 bp, Artificial Sequence
  Synthetic- Stat1 F
aggggccatcacattcacat

SEQ ID NO: 10 - DNA, 20 bp, Artificial Sequence
  Synthetic- Stat1 R
agatacttcaggggattctc

SEQ ID NO: 11 - DNA, 20 bp, Artificial Sequence
  Synthetic- Trp53ip1 F
tcctcagcagagcacacttc

SEQ ID NO: 12 - DNA, 20 bp, Artificial Sequence
  Synthetic- Trp53ip1 R
tccattggacaggactcaaa

SEQ ID NO: 13 - DNA, 20 bp, Artificial Sequence
  Synthetic- Cdc6 F
agggtgactttgagccaaga

SEQ ID NO: 14 - DNA, 20 bp, Artificial Sequence
  Synthetic- Cdc6 R
atgaagattctgggggctct

SEQ ID NO: 15 - DNA, 20 bp, Artificial Sequence
  Synthetic- E2f1 F
tgcagaaacggcgcatctat

SEQ ID NO: 16 - DNA, 19 bp, Artificial Sequence
  Synthetic- E2f1 R
ccgcttaccaatccccacc

SEQ ID NO: 17 - DNA, 21 bp, Artificial Sequence
  Synthetic- Pcna F
ttgcacgtatatgccgagacc

SEQ ID NO: 18 - DNA, 23 bp, Artificial Sequence
  Synthetic- Pcna R
ggtgaacaggctcattcatctct

SEQ ID NO: 19 - DNA, 20 bp, Artificial Sequence
  Synthetic- Mcm5 F
ggaggctattgtgcgcattg

SEQ ID NO: 20 - DNA, 20 bp, Artificial Sequence
  Synthetic- Mcm5 R
ctggtcctcctgggtagtga

SEQ ID NO: 21 - DNA, 20 bp, Artificial Sequence
  Synthetic- Ccne2 F
tctgtgcattctagccatcg

SEQ ID NO: 22 - DNA, 20 bp, Artificial Sequence
  Synthetic- Ccne2 R
acaaaaggcaccatccagtc

SEQ ID NO: 23 - DNA, 21 bp, Artificial Sequence
  Synthetic- Reelin F
ggactaagaatgcttatttcc

SEQ ID NO: 24 - DNA, 22 bp, Artificial Sequence
  Synthetic- Reelin R
ggaagtagaattcatccatcag

SEQ ID NO: 25 - DNA, 19 bp, Artificial Sequence
  Synthetic- Rlp32 F
gcctctggtgaagcccaag

SEQ ID NO: 26 - DNA, 22 bp, Artificial Sequence
  Synthetic- Rlp32 R
ttgttgctcccataaccgatgt

SEQ ID NO: 27 - DNA, 19 bp, Artificial Sequence
  Synthetic- Reelin F
caatctgaatggcgaaacc

SEQ ID NO: 28 - DNA, 24 bp, Artificial Sequence
  Synthetic- Reelin R
ctttcgctataaatcggagagaga

SEQ ID NO: 29 - DNA, 20 bp, Artificial Sequence
  Synthetic- Gapdh F
tgaccacagtccatgccatc

SEQ ID NO: 30 - DNA, 20 bp, Artificial Sequence
  Synthetic- Gapdh R
gacggacacattgggggtag

SEQ ID NO: 31 - DNA, 19 bp, Artificial Sequence
  Synthetic- MMRN1 F
ttggattggaggtgctgtc

SEQ ID NO: 32 - DNA, 19 bp, Artificial Sequence
  Synthetic- MMRN1 R
gcctggttggtgtgtatca

SEQ ID NO: 33 - DNA, 19 bp, Artificial Sequence
  Synthetic- THBS1 F
caccaaccgcattccagag

SEQ ID NO: 34 - DNA, 19 bp, Artificial Sequence
  Synthetic- THBS1 R
tcagggatgccagaaggag

SEQ ID NO: 35 - DNA, 20 bp, Artificial Sequence
  Synthetic- HSPG2 F
ctccatcgtcatctccgtct

SEQ ID NO: 36 - DNA, 19 bp, Artificial Sequence
  Synthetic- HSPG2 R
gtctgcccttctgccactc

SEQ ID NO: 37 - DNA, 20 bp, Artificial Sequence
  Synthetic- FN1 F
ccatcgcaaaccgctgccat

SEQ ID NO: 38 - DNA, 22 bp, Artificial Sequence
  Synthetic- FN1 R
aacacttctcagctatgggctt

SEQ ID NO: 39 - DNA, 20 bp, Artificial Sequence
  Synthetic- FSTL1 F
cgatggacactgcaaagaga

SEQ ID NO: 40 - DNA, 20 bp, Artificial Sequence
  Synthetic- FSTL1 R
ccagccatctggaatgatct

SEQ ID NO: 41 - DNA, 16 bp, Artificial Sequence
  Synthetic- LAMA4 F
gcggccgagaaatgca

SEQ ID NO: 42 - DNA, 19 bp, Artificial Sequence
  Synthetic- LAMA4 R
agtcgcagggcacacattc

SEQ ID NO: 43 - DNA, 32 bp, Artificial Sequence
  Synthetic- SERPINE1 F
acaagttcaactatactgagttcaccacgccc

SEQ ID NO: 44 - DNA, 30 bp, Artificial Sequence
  Synthetic- SERPINE1 R
tgaaactgtctgaacatgtcggtcattccc

(19) United States
(12) Patent Application Publication
(10) Pub. No.: US 2023/0390359 A1
(43) Pub. Date: Dec. 7, 2023

(54) USE OF REELIN FOR TREATING CARDIAC DISEASES

(71) Applicant: Northwestern University, Evanston, IL (US)

(72) Inventors: Guillermo Oliver, Chicago, IL (US); Xiaolei Liu, Chicago, IL (US)

(21) Appl. No.: 18/249,057

(22) PCT Filed: Oct. 14, 2021
(86) PCT No.: PCT/US21/55028

§ 371 Date: Apr. 13, 2023

Related U.S. Application Data
(60) Provisional application No. 63/091,558, filed on Oct. 14, 2020.

(51) Int. Cl. A61K 38/17; A61L 15/32; A61P 9/00

(52) U.S. Cl. CPC A61K 38/1709; A61L 15/325; A61P 9/00; A61L 2300/252

(57) ABSTRACT

Disclosed herein are compositions and methods useful for treating diseases, conditions and injuries of the heart, and/or improving cardiac function in a subject in need thereof. In some embodiments, the methods comprise administering a therapeutically effective amount of a reelin polypeptide, a functional fragment or variant thereof, or a nucleic acid that expresses a reelin polypeptide or a functional fragment or variant thereof, to the subject.

### CROSS-REFERENCE TO RELATED APPLICATIONS

This application claims the benefit of U.S. Provisional Application No. 63/091,558, filed Oct. 14, 2020, the contents of which is incorporated herein by reference in its entirety.

### STATEMENT REGARDING FEDERALLY SPONSORED RESEARCH OR DEVELOPMENT

This invention was made with government support under HL073402 awarded by the National Institutes of Health. The government has certain rights in the invention.

### SEQUENCE LISTING

A Sequence Listing accompanies this application and is submitted as an ASCII text file of the sequence listing named “702581_2038_ST25.txt” which is 68 KB in size and was created on Oct. 4, 2021. The sequence listing is electronically submitted via EFS-Web with the application and is incorporated herein by reference in its entirety.

### BACKGROUND

Over the past decades, the molecular and functional characterization of the lymphatic vasculature in normal and pathophysiological conditions has greatly improved. Recent data suggest that natural or therapeutic formation of new lymphatics (lymphangiogenesis) correlates with improved systolic function after experimental myocardial infarction (MI); it delays atherosclerotic plaque formation, facilitates the healing process after MI, and can be a natural response to fluid accumulation into the myocardium during cardiac edema. These new findings argue that specific stimulation of lymphangiogenesis in the infarcted heart could be a valuable therapeutic approach to improve cardiac function and prevent adverse cardiac remodeling. Studies in mouse and zebrafish suggested that newly formed lymphatics provide a route for the clearance of immune cells in the injured heart, and therefore promote cardiac repair. However, whether lymphatics could have additional functional roles during heart development and regeneration is not yet known, nor is it known how cardiac lymphatics improve cardiac repair.

### SUMMARY

Disclosed herein are compositions and methods useful for treating diseases, conditions and injuries of the heart, and/or improving cardiac function in a subject in need thereof. In some embodiments, the methods comprise administering a therapeutically effective amount of a reelin protein, a functional fragment or variant thereof, or a nucleic acid that expresses a reelin polypeptide or a functional fragment or variant thereof, to the subject. In some embodiments, the subject is at risk of, or has suffered a cardiac injury, or is at risk of or has been diagnosed with a cardiac disease. In some embodiments, the cardiac injury, condition, or disease includes but is not limited to: (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) and endocarditis (bacterial, viral, or fungal).

In some embodiments disclosed herein, therapeutic compositions are disclosed. In some embodiments, a therapeutic composition includes a reelin polypeptide, or a functional fragment or variant thereof, embedded in a collagen matrix or in nanoparticles or viral particles.

### BRIEF DESCRIPTION OF THE DRAWINGS

FIG. 1a-j. Lymphatics are required for embryonic heart growth. a, Wild type mouse cardiac lymphatic vasculature development as depicted by anti-Lyve1 whole mount immunostaining. Yellow arrowheads indicate cardiac lymphatics at E14.5. b-c, Bright field images of E17.5 control and Prox1^{ΔLEC/ΔLEC} embryos and hearts. White arrow indicates edema in Prox1^{ΔLEC/ΔLEC} embryos. d-i, Whole mount immunostaining shows that E17.5 Prox1^{ΔLEC/ΔLEC} hearts lack Lyve1+ cardiac lymphatics and have normal major coronary arteries and veins, as indicated by α-SMA and endomucin (EMCN) staining. Arrowheads indicate developing lymphatics in control hearts. j, Quantification of organ weight relative to body length (BL) shows reduced heart size and normal liver and kidney sizes in E17.5 Prox1^{ΔLEC/ΔLEC} embryos (N=13 controls and N=10 Prox1^{ΔLEC/ΔLEC} embryos; 3 different litters). Data is presented as mean+S.E.M. ***p=3.19062E-06 by unpaired two-tailed Student's t test. n.s, not significant. Control embryos are TAM treated Cre⁻ and Cre⁺; Prox1^{+/+} littermates. HW, heart weight; LW, liver weight; KW, kidney weight. N+3 embryos/genotype, d-i). Scale bars, 500 μm c-i), 2 mm (b).

FIG. 2a-h. Lymphatics are required for CM proliferation and survival. a, H&E staining shows no obvious defects in cardiac valves (arrows) or ventricular wall compaction in E17.5 Prox1^{ΔLEC/ΔLEC} hearts (TAM injected at E13.5 and E14.5). N=4 embryos/genotype. b, α-Laminin staining shows no differences in Prox1+ CM size between E17.5 controls and Prox1^{ΔLEC/ΔLEC} hearts. Right panel shows quantification of Prox1+ CM size (α-Laminin+ area). Average cell size was measured from five fields/ventricle, 8-10 Prox1+ CMs/field, 3 embryos per genotype; N=152 (control) and 155 (Prox1^{ΔLEC/ΔLEC}). c-f, Immunostaining with proliferation markers (EdU, pH3, Ki67 and AuroraB) together with CM markers (cardiac Troponin C [cTnC], Prox1, αActinin and/or Mef2c). In all images, arrows indicate the double positive CMs selected for counting. N=4 embryos/genotype from 3 separate litters. g, Quantification of the immunostaining in c-f shows reduced number of EdU+, Ki67+, AuroraB+ and pH3+ CMs in E17.5 Prox1^{ΔLEC/ΔLEC} hearts. N=4 embryos/genotype from 3 separate litters. **p=0.003 (EdU, Ki67 and AuroraB), **p=0.002 (pH3). h, Active Caspase-3 immunostaining shows increased CM apoptosis in Prox1+ CMs in E17.5 Prox1^{ΔLEC/ΔLEC} hearts. Arrows indicate apoptotic CMs. N=4 embryos/genotype from 3 separate litters. ***p=0.0003. Control embryos are TAM treated Cre⁻ and Cre⁺; Prox1^{+/+} littermates. Data are presented as mean±S.E.M. p values were calculated by unpaired two-tailed Student's t test. n.s, not significant. Scale bars, 1 mm (a), 25 μm (b, c-f, h). Lower magnification of panels c-e and h are included in FIG. 16.

FIG. 3a-i. LECs-secreted Reelin promotes CM proliferation and survival. a, b, Quantitative Western blot results show increased p-AKT and p-ERK in LEC-conditioned media-treated hiPSC-CMs (a) and mouse primary CMs (b). *p=0.012 (p-AKT, a), *p=0.015 (p-ERK, a), *p=0.013 (p-AKT and p-ERK, b). N=4 (a) and N=3 (b). c, Bright field images of E17.5 Reln^{ΔLEC/ΔLEC} and control embryos and hearts (TAM injected at E13.5 and E14.5). Quantification of organ weight heart (HW), liver (LW) and kidney (KW) relative to body length (BL) indicates that hearts are smaller in Reln^{ΔLEC/ΔLEC} embryos. N=22 (controls) and N=11 (Reln^{ΔLEC/ΔLEC}) from 5 litters. *p=0.016. Controls are TAM treated Cre⁻ embryos and Cre⁺; Reln^{+/+} littermates. d-g, Double immunostaining using proliferation (EdU, pH3, Ki67 and AuroraB) and CM (cardiac Troponin C [cTnC], Prox1, αActin and/or Mef2c) markers shows reduced CM proliferation in E17.5 Reln^{ΔLEC/ΔLEC} hearts. Arrows indicate proliferating CMs. h, Quantification of the immunostaining in d-g shows reduced number of EdU+, Ki67+, AuroraB+ and pH3+ CMs in E17.5 Reln^{ΔLEC/ΔLEC} hearts. N=4 embryos/genotype from 3 separate litters. *p=0.02 (EdU), *p=0.01 (Ki67), **p=0.001 (pH3) and *p=0.035 (AuroraB). i, Active Caspase-3 immunostaining shows increased CM apoptosis (arrows) in E17.5 Reln^{ΔLEC/ΔLEC} hearts. Right panel shows quantification of the percentage of active caspase-3+ CMs in E17.5 control and Reln^{ΔLEC/ΔLEC} hearts. N=4 embryos/genotype from 3 separate litters. **p=0.002. Control embryos are TAM treated Cre⁻ embryos and Cre⁺; Reln^{+/+} littermates. Data are presented as Mean±S.E.M. p values were calculated by unpaired two-tailed Student's t test. n.s, not significant. Scale bars, 1 mm (c), 25 μm (d-g, i). Lower magnification for panels d-f and i are included in FIG. 17. For western blot source data, see FIGS. 20 and 21.

FIG. 4. Reelin improves neonatal and adult cardiac function after myocardial Infarction. a, Echocardiography reveals relatively normal cardiac function at P7 and reduced at P14 and P21 in Reln^{-/-} mice after MI at P2. P7: N=9 (WT), N=9 (Reln^{+/-}), N=10 (Reln^{-/-}); P14: N=10 (WT), N=8 (Reln^{+/-}), N=7 (Reln^{-/-}); P21: N=9 (WT), N=13 (Reln^{+/-}), N=8 (Reln^{-/-}). *p=0.04 (P14) and *p=0.012 (P21) by two-way ANOVA followed by Bonferroni test. b, Masson's trichrome staining shows increased fibrosis in P21 Reln^{-/-} hearts (MI at P2). Right panel, quantification of the percentage of fibrotic area. N=6 (WT), N=4 (Reln^{+/-}), N=5 (Reln^{-/-}). **p=0.002 by one-way ANOVA followed by Tukey's test. c, CM proliferation is decreased in the border of the infarcted area of P7 Reln−/− hearts (N=4 mice/genotype). *p=0.026 (EdU), *p=0.025 (Ki67), *p=0.022 (pH3) and *p=0.023 (AuroraB) by unpaired two-tailed Student's t test. d, CM apoptosis increases significantly in the infarcted area of P7 Reln−/− hearts (N=4 mice/genotype). *p=0.032 by unpaired two-tailed Student's t test. e, Sutured collagen patch onto the adult mouse heart following MI. f, Residual collagen patch remains up to 42 days after MI. g, Echocardiography reveals significantly improved cardiac function (EF %) in adult mice with REELIN patches starting at around 21 days after MI and up to 42 days post-MI. N=6 (sham), N=6 (control patch) and N=7 (REELIN patch). *p=0.04 (P21), **p=0.001 (P35) and *p=0.01 (P42) by two-way ANOVA followed by Bonferroni test. h, Masson's trichrome staining shows reduced cardiac fibrotic area in REELIN patch-treated mice 42 days post-MI. Arrowhead shows fibrotic area; arrows indicate residual collagen patch. Right panel, quantification of the percentage of fibrotic area. N=4 (sham), N=4 (control patch), N=6 (REELIN patch). **p=0.003 by one-way ANOVA followed by Tukey's test. i, No differences in CM proliferation between control patch and REELIN patch treated hearts were observed in the infarcted areas 7 days post-MI (N=4 hearts/group). n.s. not significant difference by unpaired two-tailed Student's t test. j, CM apoptosis is reduced in the infarcted area of REELIN patch treated hearts (N=4 mice/group). *p 0.039 by unpaired two-tailed Student's t test. Data are presented as Mean±S.E.M. Scale bars, 500 μm (b, h). Representative images of c, d, i and j are in FIG. 15.

FIG. 5a-g. E17.5 Prox1^{ΔLEC/ΔLEC} hearts lack LECs and have a reduced number of CMs. a, Whole mount immunostaining with anti-Prox1 antibody shows that cardiac lymphatics are missing in E17.5 Prox1^{ΔLEC/ΔLEC} hearts (TAM injected at E13.5 and E14.5). Squared areas are shown in larger magnification in the adjacent images. N=3 embryos/group from two litters. b, Co-immunostaining of E17.5 control and Prox1^{ΔLEC/ΔLEC} heart sections with anti-α-Actinin and F-actin antibodies show that cardiac muscle is not affected in Prox1^{ΔLEC/ΔLEC} embryos (TAM injected at E13.5 and E14.5). N=3/group. c, Flow cytometry analysis shows reduced CM numbers in E17.5 Prox1^{ΔLEC/ΔLEC} hearts (TAM injected at E13.5 and E14.5). d, Hoechst 33342 labeling shows no significant differences in CMs ploidy between control and Prox1^{ΔLEC/ΔLEC} hearts. N=3 (control) and N=4 (Prox1^{ΔLEC/ΔLEC}) embryos from the same litter used in c and d. Data are presented as mean±S.E.M. *p=0.001 and n.s (not significant) were calculated by unpaired two-tailed Student's t test. e-g, The percentage of multinucleated CMs in E17.5 mutant hearts is increased (e, f), and no global differences in CM size were detected (e, g) after CM dissociation and o/n plating. N=3 embryo/genotype from the same litter. White arrows indicate CMs and yellow arrows indicate a bi-nucleated CM (g). The average cell size was calculated from 25 cTnC+ CMs/culture (1 whole heart/culture; 3 cultures per genotype). N=75 (control CMs) and N=75 (Prox1^{ΔLEC/ΔLEC} CMs). Data are presented as mean±S.E.M. p=0.023 and n.s (not significant) as calculate by unpaired two-tailed Student's t test. Scale bars, 500 μm (a), 25 μm (b, e). Flow cytometry gating strategy is included in FIG. 25.

FIG. 6a-g. CM proliferation is reduced in E17.5 Prox1^{ΔLEC/ΔLEC} hearts. a. EdU labeling shows an overall reduction in the number of EdU+ cells in sections of E17.5 Prox1^{ΔLEC/ΔLEC} hearts. Dashed boxes indicate the corresponding areas of the heart that are shown at higher magnification in panels b-e. b-e, Immunostaining results show the presence of Prox1+Lyve1+ cardiac lymphatics (white arrows) in sections of control hearts (b, c), and lack of lymphatics in Prox1^{ΔLEC/ΔLEC} hearts (d, e). Yellow arrows indicate Lyve1+Prox1− macrophages. N=3 embryos/genotype from 3 separate litters. f, CM proliferation is reduced in the myocardium of the left ventricle area (LV), the right ventricle area (RV) and the septum. N=4 embryos/genotype from 3 separate litters. At least 3 images/region and 3 separate regions/heart were quantified. Data are presented as mean±S.E.M. *p=0.01, **p=0.003, 0.006 and *p=0.02 (upper panel); ***p=0.0001, **p=0.004, 0.002 and 0.005 (middle panel); **p=0.001, 0.001, *p=0.01 and **p=0.002 (bottom panel) as calculated by unpaired two-tailed Student's t test. g, Immunostaining with antibodies against Vimentin (fibroblasts), PECAM1 (blood endothelial cells), CD68 (macrophages), Six2 (nephron progenitors) or Hnf4α (hepatocytes) together with EdU labeling (white arrows) shows no differences in proliferation in those cell types between E17.5 Prox1^{ΔLEC/ΔLEC} and control hearts (TAM injected at E13.5 and E14.5). n.s. no significant differences by unpaired two-tailed Student's t test. N=3 embryos/genotype from 3 separate litters. Control are TAM treated Cre− embryos and Cre+; Prox1+/+ littermates. Data are presented as mean±S.E.M. Scale bars, 200 μm (a), 100 μm (b-e), 25 μm(g).

FIG. 7a-i. Vegfr3^{kd/kd} embryos lack cardiac lymphatics and have smaller hearts. a. Bright field images of whole E17.5 Vegfr3kd/kd and WT embryos and hearts. Quantification of organ weight (heart, liver and kidney) relative to body length indicates that the heart is smaller and the liver and kidney have comparable sizes between Vegfr3kd/kd and control embryos. N=10 (WT) and N=8 (Vegfr3^{kd/kd}). Embryos are from 3 different litters. *p=0.019. b, Lyve1 whole mount immunostaining shows that ventral and dorsal sides of the heart are devoid of lymphatics in Vegfr3^{kd/kd} embryos. N=3/genotype. c-f, Co-immunostaining using antibodies against cell proliferation markers (EdU, pH3, Ki67 and AuroraB) and antibodies against CM markers (cardiac Troponin C [cTnC], Prox1, αActinin and/or Mef2c) shows reduced CM proliferation in Vegfr3^{kd/kd} hearts compared to wild-type hearts at E17.5. Arrows indicate representative proliferating CMs. g, Quantification shows significantly reduced percentage of EdU+ and Ki67+ CMs and significantly reduce number of pH3+ and AuroraB+ CMs in Vegfr3kd/kd hearts compared to controls. N=4 embryos/genotype from 3 separate litters. **p=0.005 (EdU), 0.001(Ki67, pH3), *p=0.02 (AuroraB). h, Active Caspase-3 immunostaining shows increased CM apoptosis (white arrows) in Vegfr3kd/kd hearts compared to wild-type hearts at E17.5. Right panel is the quantitative data showing significantly increased percentage of active caspase-3+ CMs (Prox1+) in Vegfr3kd/kd hearts compared to wild-types. N=4 embryos/genotype from 3 separate litters. *p=0.032. i, Co-immunostaining with antibodies against Vimentin, PECAM1, CD68, Six2 and Hnf4α, together with EdU labeling shows comparable proliferation of cardiac fibroblasts, blood endothelial cells and macrophages, and of nephron progenitors and hepatocytes between wild-type and Vegfr3kd/kd embryos at E17.5. White arrows indicate EdU+ proliferating cells. Quantification of the proliferation for each of those cell types is shown on the right panels. n.s. not significant. N=3 embryos/genotype from 3 separate litters. Data are presented as mean±S.E.M. p values were calculated by unpaired two-tailed Student's t test. Scale bars, 1 mm(a), 500 μm (b), 25 μm (c-f, h), 25 μm (i). Lower magnification images for panels c-e and h are included in FIG. 19.

FIG. 8a-h. Heart size and CM proliferation is normal in E17.5 Prox1^{ΔLEC/+} embryos and E14.5 Prox1^{ΔLEC/ΔLEC} embryos. a, Bright field images of whole embryos and hearts show no difference in heart size in E17.5 Prox1^{ΔLEC/+} embryos (TAM injected at E13.5 and E14.5). White arrows indicate edema in the Prox1^{ΔLEC/+} embryo. b, Whole mount immunostaining shows that cardiac lymphatics are present in both dorsal and ventral sides of Prox1^{ΔLEC/+} hearts. Lymphatics are less branched (arrows). c, Cardiac lymphatic density is significantly reduced on the ventral surface of the heart but not on the dorsal one in Prox1^{ΔLEC/+} embryos. This difference may be because cardiac lymphatics on the dorsal side and the ventral side originate from two different lineages during embryonic development. N=3 embryos/genotype from 3 separate litters. *p=0.027. d, Heart size is normal in E17.5 Prox1^{ΔLEC/+} embryos. N=13 (controls) and N=9 (Prox1^{LEC/+}) embryos from 3 separate litters. e, Quantification of the immunostaining analysis shows no significant differences in CM proliferation between E17.5 Prox1^{ΔLEC/+} hearts and controls, as indicated by the percentage of EdU+ and Ki67+ CMs and the number of pH3+ and AuroraB+ CMs. N=4 embryos/genotype from 3 separate litters. Controls are TAM treated Cre− embryos and Cre+; Prox1+/+littermates. f, Bright field images of whole embryos and hearts show no difference in cardiac size in between E14.5 wild-type and Prox1^{ΔLEC/ΔLEC} embryos (TAM injected at E10.5 and E11.5). White arrows indicate severe edema. N=6 embryos/genotype from 2 separate litters. Control embryos are TAM treated Cre embryos and Cre+; Prox1+/+littermates. g, Whole mount staining of skin shows efficient Prox1 deletion as indicated by the lack of Prox1+ or Nrp2+ lymphatics at E14.5 in Prox1^{ΔLEC/ΔLEC} embryos. N=3 embryos/genotype from same litter. h, Co-immunostaining against cell proliferation markers (EdU, Ki67, pH3 and AuroraB) together with CM markers (cardiac Troponin C [cTnC], Prox1, α-Actinin and/or Mef2c). Quantification of those immunostainings shows no differences in CM proliferation between wild-type and Prox1^{ΔLEC/ΔLEC} hearts at E14.5. Squares indicate proliferating CMs. N=3 embryos/genotype from the same litter. Data are presented as mean±S.E.M. n.s. not significant difference by unpaired two-tailed Student's t test. Scale bars, 1 mm (a, f), 500 μm (b), 25 μm (g, h).

FIG. 9a-d. Pathways related to cell cycle are downregulated in E17.5. Prox1^{ΔLEC/ΔLEC} embryos and LECs-conditioned media promotes CM proliferation and survival in vitro. a, GSEA shows downregulation of cell cycle pathways and upregulation of cell death pathways in Prox1^{ΔLEC/ΔLEC} hearts. N=4/genotype from the same litter. b, qPCR analysis confirmed the upregulation of pro-apoptotic genes (Bcl12ll, Pdcd4, Trp53ip, Stat1 and P21) and downregulation of cell cycle related genes (Cdc6, E2f1, Pcna, Mcm3 and Ccne2) in Prox1^{ΔLEC/ΔLEC} hearts. N=3/genotype from the same litter. TAM was injected at E13.5 and E14.5. Control embryos are TAM treated Cre− embryos and Cre⁺; Prox1^{+/+} littermates. *p=0.02 (Bcl12ll), **p=0.001 (Pdcd4), 0.005 (Trp53ip), *p=0.01 (Stat1), 0.03 (P21), 0.04 (Cdc6), 0.02 (E2f1), 0.01 (Pcna), 0.02 (Mcm5) and 0.03 (Ccne2). c, Coimmunostaining against the proliferation marker Ki67 and the CM markers α-Actinin and Prox1 shows that LECs-conditioned media increases primary CM proliferation. Arrows indicate proliferating CMs. Percentage of CM proliferation was quantified by the number of Ki67+ Prox1+ CMs relative to total number of Prox1+ CMs. N=3. **p=0.001. d, Co immunostaining against the apoptotic marker active Caspase-3 and the CM markers α-Actinin and Prox1 shows reduced primary CM apoptosis upon LEC-conditioned media treatment under CoCl₂ induced hypoxia. Arrows indicate apoptotic CMs. Percentage of apoptotic CMs was quantified by the number of active Caspase 3+ CMs relative to Prox1+ CMs. N=3. **p=0.003. Data are presented as mean±S.E.M. p values were calculated by unpaired two-tailed Student's t test. n.s, not significant. Scale bar, 25 μm (c,d).

FIG. 10a-i. E17.5 Reln^{-/-} embryos develop smaller hearts. a, qPCR analysis shows reduced Reln expression in E17.5 Prox1^{ΔLEC/ΔLEC} hearts (TAM injected at E13.5 and E14.5). N=3 embryos/genotype from the same litter. Control embryos are TAM treated Cre− embryos and Cre+; Prox1_{+/+}littermates. *p=0.014. b, qPCR analysis validates the expression of candidates from the LECs secretome (SERPINE1, FN, RELN, HSPG2, MMRN1, LAMA4, FSTL1 and THBSI). Experiments were repeated 3 times using different batches of LECs. Gene expression is normalized as a fold change relative to 100× Gapdh. c, Reelin protein can be detected in 3 different batches of LEC conditioned media and the relative Reelin level is quantified by ELISA according to the OD intensity. d-e, Immunostaining of sections of E17.5 WT hearts shows Reelin is highly expressed in cardiac lymphatics of the epicardium and myocardium. Some blood vessels in the heart express low levels of Reelin (e, arrows). N=3 WT embryos. f, Immunostaining of E17.5 control and Prox1^{ΔLEC/ΔLEC} heart sections with antibodies against Reelin and Lyve1 shows that cardiac lymphatics and Reelin are absent in Prox1^{ΔLEC/ΔLEC} hearts (TAM injected at E13.5 and E14.5). N=3 embryos/genotype from the same litter. Control embryos are TAM treated littermate Cre− and Cre+; Prox1^{+/+} embryos. g, Representative bright field images show smaller hearts in E17.5 Reln^{-/-} embryos. h, Quantifications of organ weight (heart, liver and kidney) relative to body length indicate that hearts are smaller in E17.5 Reln-i embryos compared to controls. N=7 (WT) and N=6 (Reln^{-/-}) embryos from 3 separate litters. *p=0.03. i, Whole mount immunostaining shows that cardiac lymphatic development is normal in Reln-i embryos. N=3 embryos/genotype from 2 separate litters. Data are presented as mean±S.E.M. p values were calculated by unpaired two-tailed Student's t test. n.s, not significant. Scale bar, 25 μm (d, e, f), 1 mm (g), 500 μm (i).

FIG. 11a-b. Reelin is efficiently deleted in Reln1^{ΔLEC/ΔLEC} cardiac associated. Lymphatics. a, Immunostaining of E17.5 control and Reln1^{ΔLEC/ΔLEC} heart sections with antibodies against Reelin and Lyve1 confirms that Reelin is deleted from cardiac lymphatics in Reln1^{ΔLEC/ΔLEC} hearts (TAM injected at E13.5 and E14.5). N=3 embryos/genotype from 2 separate litters. Control embryos are TAM treated Cre− embryos and Cre+; Reln_{+/+} embryos. b, Co-immunostaining with antibodies against Vimentin, PECAM1, CD68, Six2 and Hnf4a, together with EdU labeling shows comparable proliferation of cardiac fibroblasts, blood endothelial cells and macrophages, and of nephron progenitors and hepatocytes between controls and E17.5 Reln1^{ΔLEC/ΔLEC} hearts (TAM injected at E13.5 and E14.5). White arrows indicate EdU+ proliferating cells. Quantification of the proliferation for each of those cell types is shown on the right panels. N=3 embryos/genotype from 2 separate litters. Control embryos are TAM treated Cre− and Cre+; Reln_{+/+} littermates. Data are presented as mean±S.E.M. n.s. not significant difference by unpaired two-tailed Student's t test. Scale bar, 25 μm.

FIG. 12a-g. Cardiac size is reduced in E17.5 β1^{ΔCM/+}; Rel^{+/-} embryos. a, qPCR analysis shows efficient Reln knockdown in LECs after siRNA treatment. N=3. Mean±S.E.M. p<0.05 by unpaired two-tailed Student's t test. b, Representative Western blot of primary CMs cultured with DMEM, siCtrl and siReln treated conditioned media, or with conditioned media+Integrinol blocking antibody o/n. Addition of the LEC conditioned media (siCtrl group) to primary CMs increases Dab1, FAK, AKT and ERK activities. These activities are reduced when cultured CMs are treated with Reelin deficient LECs conditioned media or with LECS conditioned media with β1 blocking antibody. Experiments were repeated 3 times. Data are presented as mean±S.E.M. *p<0.05; **p<0.01; ***p<0.001 by two-way ANOVA followed by Bonferroni test. c, Ki67 quantification of immunostained cultured cells (similar to Extended Data FIG. 5c) shows that addition of the LEC conditioned media (siCtrl group) to cultured primary CMs improves CM proliferation and this effect is partially abolished in CMs treated with Reln (siReln) deficient LECs conditioned media or with LECs conditioned media containing 01 blocking antibody. Percentage of CM proliferation was quantified by the number of Ki67+Prox1+CMs relative to total numbers of Prox1+CMs. N=3. Mean±S.E.M. **p<0.01 by two-way ANOVA followed by Bonferroni test. d, Quantification of active Caspase 3 immunostained cultured CMs shows that addition of the LEC conditioned media (siCtrl group) to primary CMs protect them from apoptosis and this effect is partially abolished in CMs treated with Reln deficient LECs conditioned media or with LECs conditioned media with β1 blocking antibodies. Percentage of apoptotic CMs was quantified by the number of active Caspase 3+CMs relative to Prox1+CMs. N=3. Mean±S.E.M. *p<0.05; **p<0.01 by two-way ANOVA followed by Bonferroni test. e, Representative Western blot of primary CMs after treatment with Reelin conditioned media from Reelin transfected cells (Reelin), or conditioned media from mock-transfected cells (control) or Reelin conditioned media with Integrin β1 blocking antibody (reelin+β1 blocking ab) shows that Reelin treatment increases Dab1, FAK, AKT and ERK activities in primary CMs, and these activities are reduced by adding the Integrin β1 blocking antibody. N=3. Data are presented as mean±S.E.M. *p=0.05; **p<0.01 by one-way ANOVA followed by Tukey's test. f, Bright field images show no difference in embryo size at E17.5 among control, Reln+/−, β1^{ΔCM/+} and β1^{ΔCM/+}; Reln+/− embryos. Quantification of organ weight (heart, liver and kidney) relative to body length indicates that hearts are smaller in E17.5 β1^{ΔCM/+}; Reln^{+/-} embryos. N=9 (control), N=7 (Reln+/−), N=6 (β1^{ΔCM/+}) and N=6 (β1^{ΔCM/+}; Reln+/−) embryos from 3 separate litters. Data are presented as mean±S.E.M. *p=0.015 by one-way ANOVA followed by Tukey's test. n.s, not significant. g, Whole mount immunostaining using Lyve1 antibodies shows normal cardiac lymphatic development in control, β1^{ΔCM/+}, β1^{ΔCM/+}; Reln^{+/-} and Reln^{+/-} embryos. N=3 embryos/genotype from 3 separate litters. Scale bars, 1 mm (f), 500 μm (g). For western blot source data, see FIGS. 23 and 24.

FIG. 13a-c. Reelin promotes CM proliferation and survival through Integrin β1 signaling a, Co-immunostaining using cell proliferation markers (EdU, Ki67, pH3 and AuroraB) together with CM markers (cardiac Troponin C [cTnC], Prox1, αActinin and/or Mef2c) shows reduced CM proliferation in β1^{ΔCM/+}; Reln^{+/-} hearts at E17.5. Arrows indicate proliferating CMs. Quantification in the lower panel shows reduced proliferation in E17.5 β1^{ΔCM/+}; Reln^{+/-} hearts, as indicated by the percentage of EdU+ and Ki67+ CMs and the number of pH3+ and AuroraB+CMs. N=4 embryos/genotype from 3 separate litters. *p=0.022 (EdU), 0.029 (Ki67), ***p=0.0001 (pH3) and *p=0.033 (AuroraB). b, Active Caspase-3 immunostaining shows increased CM apoptosis in β1^{ΔCM/+}; Reln^{+/-} hearts at E17.5, as quantified by the percentage of active caspase-3+CMs relative to Prox1+ CMs. Arrows indicate apoptotic CMs. N=4 embryos/genotype from 3 separate litters. Control embryos are Cre− embryos and Cre+; β1^{+/+} littermates. *p=0.01. c, Co-immunostaining with antibodies against Vimentin, PECAM1, CD68, Six2 and Hnf4a, together with EdU labeling shows comparable proliferation of cardiac fibroblasts, blood endothelial cells and macrophages, and of nephron progenitors and hepatocytes between controls and E17.5 β1^{ΔCM/+}; Reln^{+/-} embryos. White arrows indicate EdU+ proliferating cells. Quantification of the proliferation analysis for each of those cell types is shown on the right panels. N=3 embryos/genotype from 3 separate litters. Control are Cre− embryos and Cre⁺; β1^{+/+} littermates. Data are presented as mean±S.E.M. n.s. not significant difference by unpaired two-tailed Student's t test. Scale bars, 25 μm. Lower magnification images for panels a and b are included in FIG. 20.

FIG. 14a-e. Reelin expression is developmentally downregulated, but is upregulated in newly formed cardiac lymphatics after myocardial infarction. a. Immunostaining with Reelin, Prox1 and PECAM shows Reelin is highly expressed in cardiac lymphatics in the epicardium and myocardium nearby the base of the heart at E17.5. Reelin expression level is gradually downregulated during development from P2 to P14. N=3 hearts/stage. Arrows indicate Prox1+ cardiac lymphatics. b, qPCR analysis using sorted cardiac lymphatics shows Reln levels are drastically downregulated in cardiac LECs during development. N=3. Reln relative level from each experiment is presented as fold changes relative to E17.5. Data are presented as mean±S.E.M. **p=0.009 (P2 vs E17.5), 0.004 (P7 vs E17.5), 0.001 (P14 vs E17.5) by one-way ANOVA followed by Tukey's test. c, Immunostaining shows Reelin expression is highly upregulated in the newly formed cardiac lymphatics in WT P7 pups (myocardial infarction was performed at P2). Notably, the pre-existing cardiac lymphatics in the noninfarcted area express low levels of Reelin. Reln−/− hearts are completely devoid of Reelin expression in both, newly formed cardiac lymphatics and pre-existing lymphatics. Arrows indicate cardiac lymphatics. N=3 hearts/group. d, Immunostaining against the pan-endothelial marker PECAM1 and the lymphatic marker Lyve1 shows normal lymphangiogenesis in WT and Reln−/− hearts 21 days after MI (MI performed at P2). N=3 hearts/group. Data are presented as mean±S.E.M. n.s. not significant difference by unpaired two-tailed Student's t test. e, EdU labeling shows no differences in LECs proliferation in WT and Reln^{-/-} hearts 21 days after MI (MI performed at P2). 3 hearts/group. Data are presented as mean±S.E.M. n.s. not significant difference by unpaired two-tailed Student's t test. Arrow indicates EdU+LECs. Scale bars, 100 μm (d), 25 μm (a,c,e).

FIG. 15a-g. Reelin improves cardio-protection in neonates and adult mice after MI. a-d, Co-immunostaining using cell proliferation markers (EdU, Ki67, pH3 and AuroraB) together with the CM markers Prox1, α-Actinin or Mef2c shows decreased CM proliferation in the border of the infarcted area of Reln^{-/-} hearts at P7. Arrows indicate proliferating CMs. N=4 mice/group. e, Immunostaining using active Caspase-3 shows increased CM apoptosis in the infarcted area of Reln−/− hearts at P7. Arrows indicate apoptotic CMs in the section. N=4 mice/group. f, Immunostaining against the cell proliferation markers EdU, Ki67 and pH3 together with the CM markers Mef2c or cTnC shows no differences in CM proliferation in the infarcted areas between control patch or REELIN patch treated hearts 7 days after MI. Arrows indicate proliferating CMs. N=4 hearts/group. g, Immunostaining using active Caspase-3 shows reduced CM apoptosis in the infarcted area of REELIN patch treated hearts. Arrows indicate apoptotic CMs. N=4 mice/group. Arrows indicate apoptotic CMs. Scale bars, 25 μm Lower magnification for panels a-c, e and g are included in FIG. 18.

FIG. 16c, d, e, h. Shows lower magnification of panels from FIG. 2c-e and h.

FIG. 17d, e, f, i. Shows lower magnification for panels from FIG. 3d-f and i.

FIG. 18a-c, e, g. Shows lower magnification for panels from FIG. 15a-c, e and g.

FIG. 19c-e, h. Shows lower magnification images for panels from FIG. 7c-e and h.

FIG. 20a-b. Shows lower magnification images for panels from FIGS. 13a and b.

FIG. 21a-b. Western blot source data, see FIG. 3.

FIG. 22a-b. Western blot source data, see FIG. 3.

FIG. 23a-c. Western blot source data, see FIG. 12.

FIG. 24a-c. Western blot source data, see FIG. 12.

FIG. 25a-c. Flow cytometry gating strategy related to the experiments described at FIG. 5.

FIG. 26. Is a Table showing the functional parameters of neonatal MI. Data presented as mean±SD. Asterix indicates significant differences between WT and Reln^{-/-} groups. *p<0.05; LV: Left ventricle; ID: Internal dimension; d: diastolic; s: systolic.

FIG. 27. Is a table showing functional parameters of adult mouse MI with control REELIN patches. Data presented as mean±SD. Asterix indicates significant differences between control patch and REELIN patch treated mice. *p<0.05; LV: Left ventricle; ID: Internal dimension; d: diastolic; s: systolic.

FIG. 28. Is a table showing oligonucleotide sequences for qpCR analysis.

FIG. 29a-b. To investigate whether lymphatic endothelial cell (LECs) derived Reelin is required for adult heart repair, Reln was conditionally deleted in LECs by crossing Reln floxed mice with VE-CadCreERT2 mice. Considering Reln is mainly expressed in LECs, but not in BECs, this cross will generate LEC-specific Reln conditional nulls VE-CadCreERT2, Reln^{f/f} (Reln^{ΔEC/ΔEC}) upon Tamoxifen injections. The data clearly show that Reln^{ΔEC/ΔEC} mice display aggravated heart function (A) and increased cardiac fibrosis (B) 4 weeks post myocardial infarction (MI). *p<0.05 by unpaired student t test.

### DETAILED DESCRIPTION

The present invention is described herein using several definitions, as set forth below and throughout the application.

As used in this specification and the claims, the singular forms “a,” “an,” and “the” include plural forms unless the context clearly dictates otherwise. For example, the term “a polypeptide fragment” should be interpreted to mean “one or more a polypeptide fragments” unless the context clearly dictates otherwise. As used herein, the term “plurality” means “two or more.”

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

The disclosed methods and compositions may be utilized to treat a subject in need thereof. A “subject in need thereof” is intended to include a subject having or at risk for developing diseases or disorders of the heart. In addition to trauma, exemplary disease or conditions that negatively affect the heart and that may be treated via the compositions and methods herein, include disease or conditions such as but not limited to: (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) and endocarditis (bacterial, viral, or fungal). In some embodiments, the disclosed methods and composition are useful to treat the heart after MI. In some embodiments, the methods and compositions disclosed herein are useful to treat (e.g., alleviate, prevent, or decrease, reduce frequency of) at least one symptom of a cardiac disease, condition, or injury in a subject in need thereof. By way of example but not by way of limitation, a subject in need thereof may be exhibiting one or more of the following symptoms: (1) arrhythmia; (2) tachycardia; (3) bradycardia; (4) chest pain (angina); (5) fainting; (6) swollen feet or ankles; (7) cyanosis; (8) dizziness; (9) decreased cardiac lymphatics; (10) cardiac fluid accumulation and/or cardiac inflammation; (11) decreased systolic function; (12) atherosclerotic plaque formation; (13) delayed cardiac healing process and cardiac repair; (14) adverse cardiac remodeling; (15) shortness of breath; (16) weakness or fatigue.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Polynucleotides

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered transcription templates include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Polypeptides

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. A “variant” of a reference polypeptide sequence may include a conservative or non-conservative amino acid substitution relative to the reference polypeptide sequence,

The disclosed peptides may include an N-terminal esterification (e.g., a phosphoester modification) or a pegylation modification, for example, to enhance plasma stability (e.g. resistance to exopeptidases) and/or to reduce immunogenicity.

A “deletion” refers to a change in a reference amino acid sequence (e.g., SEQ ID NO:1 (human reelin polypeptide sequence) or SEQ ID NO:2 (rat reelin polypeptide sequence) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence (e.g., a fusion partner) that extends the half-life of the fusion polypeptide in the tissue of interest, such as serum, plasma, or in the eye. A “variant” of a reference polypeptide sequence may include a fusion polypeptide comprising the reference polypeptide.

A “fragment” is a portion of an amino acid sequence which is identical in sequence to but shorter in length than a reference sequence (e.g., SEQ ID NO:1 or SEQ ID NO:2). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

Reelin

The disclosed methods of treatment and pharmaceutical composition utilize and/or include a reelin polypeptide or a functional fragment or variant thereof, or a nucleotide sequence encoding a reelin polypeptide or a functional fragment or variant thereof.

The amino acid sequence of human reelin is provided as SEQ ID NO: 1.

SEQ ID NO: 1

| | |
|---|---|
| 1 | mersgwarqt fllalllgat lraraaagyy |
| | prfspffflc thhgelegdg eqgevlislh |
| 61 | iagnptyyvp gqeyhvtist stffdgllvt |
| | glytstsvqa sqsiggssaf gfgimsdhqf |
| 121 | gnqfmcsvva shvshlpttn lsfiwiappa |
| | gtgcvnfmat athrgqvifk dalaqqlceq |
| 181 | gaptdvtvhp hlaeihsdsi ilrddfdsyh |
| | qlqlnpniwv ecnncetgeq cgaimhgnav |
| 241 | tfcepygpre littglnttt asvlqfsigs |
| | gscrfsysdp siivlyaknn sadwiqleki |
| 301 | rapsnvstii hilylpedak genvqfqwkq |
| | enlrvgevye acwaldnili insahrqvvl |
| 361 | edsldpvdtg nwlffpgatv khscqsdgns |
| | iyfhgnegse fnfattrdvd Istediqeqw |
| 421 | seefesqptg wdvlgavigt ecgtiesgls |
| | mvflkdgerk lctpsmdttg ygnlrfyfvm |
| 481 | ggicdpgnsh endiilyaki egrkehitld |
| | tlsyssykvp slvsvvinpe lqtpatkfcl |
| 481 | ggicdpgnsh endiilyaki egrkehitld |
| | tlsyssykvp slvsvvinpe lqtpatkfcl |
| 541 | rqknhqghnr nvwavdffhv lpvlpstmsh |
| | miqfsinlgc gthqpgnsvs lefstnhgrs |
| 601 | wsllhteclp eicagphlph stvyssenys |
| | gwnritiplp naaltrntri rwrqtgpilg |
| 661 | nmwaidnvyi gpsclkfcsg rgqctrhgck |
| | cdpgfsgpac emasqtfpmf isesfgssrl |
| 721 | ssyhnfysir gaevsfgcgv lasgkalvfn |
| | kegrrqlits fldssqsrfl qftlrlgsks |
| 781 | vlstcrapdq pgegvllhys ydngitwkll |
| | ehysylsyhe priisvelpg dakqfgiqfr |
| 841 | wwqpyhssqr edvwaideii mtsvlinsis |
| | ldftnlvevt qslgfylgnv qpycghdwtl |
| 901 | cftgdsklas smryvetqsm qigasymiqf |
| | slvmgcgqky tphmdnqvkl eystnhgltw |
| 961 | hlvqeeclps mpscqeftsa siyhaseftq |
| | wrrvivllpq ktwssatrfr wsqsyytaqd |
| 1021 | ewaldsiyig qqcpnmcsgh gscdhgicrc |
| | dqgyqgtech peaalpstim sdfenqngwe |
| 1081 | sdwqevigge ivkpeggcgv issgsslyfs |
| | kagkrqlvsw dldtswvdfv qfyiqigges |
| 1141 | ascnkpdsre egvllqysnn ggiqwhllae |
| | myfsdfskpr fvylelpaaa ktpctrfrww |
| 1201 | qpvfsgedyd qwavddiiil sekqkqiipv |
| | inptlpqnfy ekpafdypmn qmsvwlmlan |
| 1261 | egmvknetfc aatpsamifg ksdgdrfavt |
| | rdltlkpgyv lqfklnigca nqfsstapvl |
| 1321 | 1qyshdagms wflvkegcyp asagkgcegn |
| | srelseptmy htgdfeewtr itiviprsla |
| 1381 | ssktrfrwiq esssqknvpp fgldgvyise |
| | pcpsycsghg dcisgvcfcd lgytaaqgtc |
| 1441 | vsnvpnhnem fdrfegklsp lwykitgaqv |
| | gtgcgtlndg kslyfngpgk reartvpldt |
| 1501 | rnirlvqfyi qigsktsgit cikprtrneg |
| | livqysndng ilwhllreld fmsflepqii |
| 1561 | sidlpqdakt patafrwwqp qhgkhsaqwa |
| | lddvligmnd ssqtgfqdkf dgsidlqanw |
| 1621 | yriqggqvdi dclsmdtali ftenigkpry |
| | aetwdfhvsa stflqfemsm gcskpfsnsh |
| 1681 | svqlqysInn gkdwhlvtee cvpptigclh |
| | ytessiytse rfqnwkritv ylplstispr |
| 1741 | trfrwigany tvgadswaid nvvlasgcpw |
| | mcsgrgicda grcvcdrgfg gpycvpvvpl |
| 1801 | psilkddfng nlhpdlwpev ygaergning |
| | etiksgtsli fkgeglrmli srdldctntm |
| 1861 | yvqfslrfia kstpershsi llqfsisggi |
| | twhlmdefyf pqttnilfin vplpytaqtn |
| 1921 | atrfrlwqpy nngkkeeiwi vddfiidgnn |
| | vnnpvmlldt fdfgprednw ffypggnigl |
| 1981 | ycpysskgap eedsamvfvs nevgehsitt |
| | rdlnvnenti iqfeinvgcs tdsssadpvr |
| 2041 | lefsrdfgat whlllplcyh ssshvsslcs |
| | tehhpsstyy agtmqgwrre vvhfgklhlc |
| 2101 | gsvrfrwyqg fypagsqpvt waidnvyigp |
| | qceemcngqg scingtkcic dpgysgptck |
| 2161 | istknpdflk ddfegqlesd rfllmsggkp |
| | srkcgilssg nnlffnedgl rmlmtrdldl |
| 2221 | sharfvqffm rlgcgkgvpd prsqpvllqy |
| | singglswsl lqeflfsnss nvgryialei |
| 2281 | plkarsgstr lrwwqpseng hfyspwvidq |
| | iliggnisgn tvleddfttl dsrkwllhpg |
| 2341 | gtkmpvcgst gdalvfieka stryvvstdv |
| | avnedsflqi dfaascsvtd scyaieleys |
| 2401 | vdlglswhpl vrdclptnve csryhlqril |
| | vsdtinkwtr itlplppytr sqatrfrwhq |
| 2461 | papfdkqqtw aidnvyigdg cidmcsghgr |
| | ciggncvcde qwgglycddp etslptqlkd |
| 2521 | nfnrapssqn wltvnggkls tvcgavasgm |
| | alhfsggcsr llvtvdlnlt naefiqfyfm |
| 2581 | ygclitpnnr nqgvlleysv nggitwnllm |
| | eifydqyskp gfvnillppd akeiatrfrw |
| 2641 | wqprhdgldq ndwaidnvli sgsadqrtvm |
| | ldtfssapvp qherspadag pvgriafdmf |
| 2701 | medktsvneh wlfhddctve rfcdspdgvm |
| | lcgshdgrev yavthdltpt egwimqfkis |
| 2761 | vgckvsekia qnqihvqyst dfgvswnylv |
| | pqclpadpkc sgsvsqpsvf fptkgwkrit |
| 2821 | yplpeslvgn pvrfrfyqky sdmqwaidnf |
| | ylgpgcldnc rghgdclreq cicdpgysgp |
| 2881 | ncylthtlkt flkerfdsee ikpdlwmsle |
| | ggstctecgi laedtalyfg gstvrqavtq |
| 2941 | dldlrgakfl qywgrigsen nmtschrpic |
| | rkegvlldys tdggitwtll hemdyqkyis |
| 3001 | vrhdyillpe daltnttrlr wwqpfvisng |
| | ivvsgveraq waldniligg aeinpsqlvd |
| 3061 | tfddegtshe enwsfypnav rtagfcgnps |
| | fhlywpnkkk dkthnalssr eliiqpgymm |
| 3121 | qfkivvgcea tscgdlhsvm leytkdarsd |
| | swqlvqtqcl psssnsigcs pfqfheatiy |
| 3181 | nsvnssswkr itiqlpdhvs ssatqfrwiq |
| | kgeetekqsw aidhvyigea cpklcsghgy |
| 3241 | cttgaicicd esfqgddcsv fshdlpsyik |
| | dnfesarvte anwetiqggv igsgcgqlap |
| 3301 | yahgdslyfn gcqirqaatk pldltraski |
| | mfvlqigsms qtdscnsdls gphavdkavl |
| 3361 | lqysvnngit whviaqhqpk dftqaqrvsy |
| | nvplearmkg vllrwwqprh ngtghdqwal |
| 3421 | dhvevvlvst rkqnymmnfs rqhglrhfyn |
| | rrrrslrryp |

The amino acid sequence of mouse reelin is provided as SEQ ID NO: 2.

| SEQ ID NO: 2 | |
|---|---|
| 1 | mergcwapra lvlavlllla tlraraatgy |
| | yprispfffl cthhgelegd geqgevlisl |
| 61 | hiagnptyyv pgqeyhvtis tstffdgllv |
| | tglytstsiq ssqsiggssa fgfgimsdhq |
| 121 | fgnqfmcsvv ashvshlptt nlsfvwiapp |
| | agtgcvnfma tathrgqvif kdalaqqlce |
| 181 | qgapteatay shlaeihsds vilrddfdsy |
| | qqlelnpniw vecsncemge qcgtimhgna |
| 241 | vtfcepygpr eltttclntt tasvlqfsig |
| | sgscrfsysd psitvsyakn ntadwiqlek |
| 301 | irapsnvstv ihilylpeea kgesvqfqwk |
| | qdslrvgevy eacwaldnil vinsahrevv |
| 361 | lednldpvdt gnwlffpgat vkhscqsdgn |
| | siyfhgnegs efnfattrdv dlstedigeq |
| 421 | wseefesqpt gwdilgavvg adcgtvesgl |
| | slvflkdger klctpymdtt gygnlrfyfv |
| 481 | mggicdpgvs hendiilyak iegrkehial |
| | dtltyssykv pslvsvvinp elqtpatkfc |
| 541 | lrqkshqgyn rnvwavdffh vlpvlpstms |
| | hmiqfsinlg cgthqpgnsv slefstnhgr |
| 601 | swsllhtecl peicagphlp hstvysseny |
| | sgwnritipl pnaaltrdtr irwrqtgpil |
| 661 | gnmwaidnvy igpsclkfcs grgqctrhgc |
| | kcdpgfsgpa cemasqtfpm fisesfgsar |
| 721 | Issyhnfysi rgaevsfgcg vlasgkalvf |
| | nkdgrrqlit sfldssqsrf lqftlrlgsk |
| 781 | svlstcrapd qpgegvllhy sydngitwkl |
| | lehysyvnyh epriisvelp ddarqfgiqf |
| 841 | rwwqpyhssq gedvwaidei vmtsvlfnsi |
| | sldftnlvev tqslgfylgn vqpycghdwt |
| 901 | lcftgdskla ssmryvetqs mqigasymiq |
| | fslvmgcgqk ytphmdnqvk leysanhglt |
| 961 | whlvqeeclp smpscqefts asiyhaseft |
| | qwrrvtvvlp qktwsgatrf rwsqsyytaq |
| 1021 | dewaldniyi gqqcpnmcsg hgscdhover |
| | cdqgyqgtec hpeaalpsti msdfenpssw |
| 1081 | esdwgevigg evvkpeggcg vvssgsslyf |
| | skagkrqlvs wdldtswvdf vqfyiqigge |
| 1141 | saacnkpdsr eegillqysn nggiqwhlla |
| | emyfsdfskp rfvylelpaa gktpctrfrw |
| 1201 | wkpvfsgedy dqwavddiii lsekqkqvip |
| | vvnptlpqnf yekpafdypm nqmsvwlmla |
| 1261 | negmakndsf cattpsamvf gksdgdrfav |
| | trdltlkpgy vlqfklnigc tsqfsstapv |
| 1321 | llqyshdagm swfllkegcf pasaakgceg |
| | nsrelseptv yytgdfeewt ritiaiprsl |
| 1381 | assktrfrwi qesssqknvp pfgldgvyis |
| | epcpsycsgh gdcisgvcfc dlgytaaqgt |
| 1441 | cvsntpnhse mfdrfegkls plwykitggq |
| | vgtgcgtlnd grslyfnglg kreartvpld |
| 1501 | trnislvqfy iqigsktsgi tyitprarye |
| | glvvqysndn gilwhllrel dfmsflepqi |
| 1561 | isidlpreak tpatafrwwq pqhgkhsaqw |
| | algdvligvn dssqtgfqdk ldgsidlqan |
| 1621 | wyriqggqvd idclsmdtal iftenignpr |
| | yaetwdfhvs essflqwemn mgcskpfsga |
| 1681 | hgiqlqysln ngkdwqlvte ecvpptigcv |
| | hytesstyts erfqnwrrvt vylplatnsp |
| 1741 | rtrfrwiqtn ytvgadswai dnvilasgcp |
| | wmcsgrgicd sgrcvcdrgf ggpfcvpvvp |
| 1801 | lpsilkddfn gnlhpdlwpe vygaergnln |
| | getiksgtcl ifkgeglrml isrdldctnt |
| 1861 | myvqfslrfi akgtpershs illqfsvsgg |
| | vtwhlmdefy fpqttsilfi nvplpygaqt |
| 1921 | natrfrlwqp ynngkkeeiw iiddfiidgn |
| | nlnnpvllld tfdfgpredn wffypggnig |
| 1981 | lycpysskga peedsamvfv snevgehsit |
| | trdlsvnent iiqfeinvgc stdsssadpv |
| 2041 | rlefsrdiga twhlllplcy hssslvsslc |
| | stehhpssty yagttqgwrr evvhfgklhl |
| 2101 | cgsvrfrwyq gfypagsqpv twaidnvyig |
| | pqceemcygh gscingtkci cdpgysgptc |
| 2161 | kistknpdfl kddfegqles drfllmsggk |
| | psrkcgilss gnnlffnedg Irmlvtrdld |
| 2221 | lsharfvqff mrlgcgkgvp dprsqpvllq |
| | yslngglsws llqeflfsns snvgryiale |
| 2281 | mplkarsgst rlrwwqpsen ghfyspwvid |
| | qiliggnisg ntvleddfst ldsrkwllhp |
| 2341 | ggtkmpvcgs tgdalvfiek astryvvttd |
| | iavnedsflq idfaascsvt dscyaieley |
| 2401 | svdlglswhp lvrdclptnv ecsryhlqri |
| | lvsdtfnkwt ritlplpsyt rsqatrfrwh |
| 2461 | qpapfdkqqt waidnvyigd gcldmcsghg |
| | rcvqgscvcd eqwgglycde petslptqlk |
| 2521 | dnfnrapsnq nwltvsggkl stvcgavasg |
| | lalhfsggcs rllvtvdlnl tnaefiqfyf |
| 2581 | mygclitpsn rnqgvlleys vnggitwill |
| | meifydqysk pgfvnillpp dakeiatrfr |
| 2641 | wwqprhdgld qndwaidnvl isgsadqrtv |
| | mldtfssapv pqherspada gpvgriafem |
| 2701 | fledktsvne nwlfhddctv erfcdspdgv |
| | mlcgshdgre vyavthdltp tenwimqfki |
| 2761 | svgckvpeki aqnqihvqfs tdfgvswsyl |
| | vpqclpadpk csgsvsqpsv ffptegwkri |
| 2821 | typlpesltg npvrfrfyqk ysdvqwaidn |
| | fylgpgcldn cgghgdclke qcicdpgysg |
| 2881 | pncylthslk tflkerfdse eikpdlwmsl |
| | eggstctecg vlaentalyf ggstvrqait |
| 2941 | qdldlrgakf lqywgrigse nnmtschrpv |
| | crkegvlldf stdggitwtl lhemdfqkyi |
| 3001 | svrhdyillp egaltnttrl rwwqpfvisn |
| | glvvsgvera qwaldnilig gaeinpsqlv |
| 3061 | dtfddegssh eenwsfypna vrtagfcgnp |
| | sfhlywpnkk kdkthnalss reliiqpgym |
| 3121 | mqfkivvgce atscgdlhsv mleytkdars |
| | dswqlvqtqc lpsssnsigc spfqfheati |
| 3181 | ynavnssswk ritiqlpdhv sssatqfrwi |
| | qkgeetekqs waidhvyige acpklcsghg |
| 3241 | ycttgavcic desfqgddcs vfshelpsyi |
| | kdnfesarvt eanwetiqgg vigsgcgqla |
| 3301 | pyahgdslyf ngcqirgaat kpldltrask |
| | imfvlqigsp aqtdscnsdl sgphtvdkav |
| 3361 | llqysvnngi twhviaqhqp kdftqaqrvs |
| | ynvplearmk gvllrwwqpr hngtghdqwa |
| 3421 | ldhvevvlvs trkqnymmnf srqhglrhfy |
| | nrrrrslrry p |

Reelin (RELN) is a large, secreted extracellular matrix glycoprotein that helps regulate processes of neuronal migration and positioning in the developing brain by controlling cell-cell interactions. In addition to its role in early development, reelin is also produced and is active in the adult brain. In the adult brain, reelin modulates synaptic plasticity by enhancing the induction and maintenance of long-term potentiation. Reelin also stimulates dendrite and dendritic spine development and regulates the continuing migration of neuroblasts generated in adult neurogenesis sites like the subventricular and subgranular zones. It was previously shown that Reelin is also expressed and secreted by lymphatic endothelial cells and regulates collecting lymphatic vessel maturation. Reelin also plays a role in atherosclerosis by enhancing vascular inflammation.

Reelin's name comes from the abnormal reeling gait of reeler mice, which were later found to have a deficiency of this brain protein and were homozygous for mutation of the RELN gene. The primary phenotype associated with loss of reelin function is a failure of neuronal positioning throughout the developing central nervous system (CNS). The mice heterozygous for the reelin gene, while having little neuroanatomical defects, display the endophenotypic traits linked to psychotic disorders.

Not surprisingly, reelin has been implicated in pathogenesis of several brain diseases. For example, the expression of the protein has been found to be significantly lower in subjects diagnosed with schizophrenia and psychotic bipolar disorder, but the cause of this observation remains uncertain, as studies show that psychotropic medication itself affects reelin expression. Moreover, epigenetic hypotheses aimed at explaining the changed levels of reelin expression are controversial. Total lack of reelin causes a form of lissencephaly, and reelin may also play a role in Alzheimer's disease, temporal lobe epilepsy and autism. Congenital lymphedema and accumulation of chylous ascites has also been reported in patients with homozygous mutations in REELIN. At least three patients with such mutation exhibited persistent neonatal lymphedema and one has accumulation of chyle. Reelin deletion in mice has been demonstrated to result in impaired maturation of collecting lymphatic vessels, suggesting that collecting vessel dysfunction may underlie the lymphatic defects observed in patients.

Loss of Reelin protects against atherosclerosis by reducing leukocyte-endothelial cell adhesion and lesion macrophage accumulation (Ding Y, Huang L, Xian X, Yuhanna I S, Wasser C R, Frotscher M, Mineo C, Shaul P W, Herz J. Loss of Reelin protects against atherosclerosis by reducing leukocyte-endothelial cell adhesion and lesion macrophage accumulation. Sci Signal. 2016 Mar. 15; 9(419):ra29).

Reelin is found not only in the brain but also in the liver, thyroid gland, adrenal gland, Fallopian tube, breast and in comparatively lower levels across a range of anatomical regions.

Reelin (human) is composed of 3461 amino acids with a relative molecular mass of 388 kDa. It also has serine protease activity.

At the N terminus, reelin contains a 27 amino acid cleavable signal peptide and a small region of similarity with F-spondin, a protein secreted by floor plate cells in the developing neural tube. At the C terminus of reelin there is a stretch of positively charged amino acids. The main body of the protein comprises a series of eight internal repeats of 350-390 amino acids, each containing two related subdomains that flank a pattern of conserved cysteine residues known as an EGF-like motif. These cysteine-rich regions resemble those found in other extracellular proteins, whereas the flanking subdomains appear to be unique to reelin.

The final reelin domain contains a highly basic and short C-terminal region (CTR) with a length of 32 amino acids. This region is highly conserved, being 100% identical in all investigated mammals. It was thought that the CTR is necessary for reelin secretion, because the Orleans reeler mutation, which lacks a part of 8th repeat and the whole CTR, is unable to secrete the misshaped protein, leading to its concentration in cytoplasm. However, other studies have shown that the CTR is not essential for secretion itself, but mutants lacking the CTR were much less efficient in activating downstream signaling events.

Reelin is cleaved in vivo at two sites located after domains 2 and 6—approximately between repeats 2 and 3 and between repeats 6 and 7, resulting in the production of three fragments. This splitting does not decrease the protein's activity, as constructs made of the predicted central fragments (repeats 3-6) bind to lipoprotein receptors, trigger Dab1 phosphorylation and mimic functions of reelin during cortical plate development. Moreover, the processing of reelin by embryonic neurons may be necessary for proper corticogenesis.

The inventors' results show that in the heart, reelin produced by lymphatics acts mainly upon the Integrin β1 signaling pathway in cardiomyocytes, although the participation of other receptors cannot be conclusively ignored. Two types of experiments, disclosed herein, illustrate this. 1) Removal of the reelin gene results in smaller hearts at embryonic stages. Also, cardiac function was impaired after myocardial infarction (MI) at postnatal day 14 and 21 as determined by echocardiography. 2) Full length reelin protein was delivered directly into adult mouse hearts using well-established bioengineered collagen patches as a scaffold to deliver recombinant reelin protein into the heart of wild-type animals. Reelin containing patches and control patches were surgically sutured onto approximately 2-month old injured hearts immediately following acute MI. Cardiac function was evaluated weekly (1-6 weeks after MI) and ejection fraction (EF) was significantly improved in mice with reelin patches. Consistent with this improved heart function, cardiomyocyte cell death and the size of the fibrotic tissue was remarkably reduced in the reelin patched mice 42 days after MI. Accordingly, reelin protein is useful for the treatment of cardiac injury; in some embodiments, full-length reelin protein is administered. In some embodiments, one or more reelin protein fragments is used (e.g., one or more reelin isoforms). In some embodiments, the reelin protein or fragment thereof is recombinant.

Reelin's control of cell-cell interactions is thought to be mediated by binding of reelin to the two members of low density lipoprotein receptor gene family: VLDLR and the ApoER2. The two main reelin receptors seem to have slightly different roles: VLDLR conducts the stop signal, while ApoER2 is essential for the migration of late-born neocortical neurons. It also has been shown that the N-terminal region of reelin, a site distinct from the region of reelin shown to associate with VLDLR/ApoER2 binds to the alpha-3-beta-1 integrin receptor.

Reelin activates the signaling cascade of Notch-1, inducing the expression of FABP7 and prompting progenitor cells to assume radial glial phenotype. In addition, corticogenesis in vivo is highly dependent upon reelin being processed by embryonic neurons, which are thought to secrete some as yet unidentified metalloproteinases that free the central signal-competent part of the protein.

In the methods and compositions disclosed herein, reelin polypeptide, or fragments thereof, can be isolated e.g., from mammalian, yeast, or bacterial cells in culture by methods well known in the art. Recombinant reelin protein is also commercially available.

Reelin Variants

The disclosed reelin variants may be modified so as to comprise an amino acid sequence, or modified amino acids, or non-naturally occurring amino acids, such that the disclosed reelin variants cannot be said to be naturally occurring. In some embodiments, the disclosed reelin variants are modified and the modification is selected from the group consisting of acylation, acetylation, formylation, lipolylation, myristoylation, palmitoylation, alkylation, isoprenylation, prenylation, and amidation. An amino acid in the disclosed polypeptides may be thusly modified, but in particular, the modifications may be present at the N-terminus and/or C-terminus of the polypeptides (e.g., N-terminal acylation or acetylation, and/or C-terminal amidation). The modifications may enhance the stability of the polypeptides and/or make the polypeptides resistant to proteolysis.

The disclosed reelin variants may be modified to replace a natural amino acid residue by an unnatural amino acid. Unnatural amino acids may include, but are not limited to an amino acid having a D-configuration, an N-methyl-α-amino acid, a non-proteogenic constrained amino acid, or a β-amino acid.

The disclosed reelin variants may be modified in order to increase the stability of the reelin variants in the target tissue, such as the heart. For example, the disclosed peptides may be modified in order to make the peptides resistant to peptidases. The disclosed peptides may be modified to replace an amide bond between two amino acids with a non-amide bond. For example, the carbonyl moiety of the amide bond can be replaced by CH2 (i.e., to provide a reduced amino bond: —CH2-NH—). Other suitable non-amide replacement bonds for the amide bond may include, but are not limited to: an endothiopeptide, —C(S)—NH, a phosphonamide, —P(O)OH—NH—), the NH-amide bond can be exchanged by O (depsipeptide, —CO—O—), S (thioester, —CO—S—) or CH₂ (ketomethylene, —CO—CH₂—). The peptide bond can also be modified as follows: retro-inverso bond (—NH—CO—), methylene-oxy bond (—CH₂—), thiomethylene bond (—CH₂—S—), carbabond (—CH₂—CH₂—), hydroxyethylene bond (—CHOH—CH₂—) and so on, for example, to increase plasma stability of the peptide sequence (notably towards endopeptidases).

The disclosed reelin variants may include a non-naturally occurring N-terminal and/or C-terminal modification. For example, the N-terminal of the disclosed peptides may be modified to include an N-acylation or a N-pyroglutamate modification (e.g., as a blocking modification). The C-terminal end of the disclosed peptides may be modified to include a C-amidation. The disclosed peptides may be conjugated to carbohydrate chains (e.g., via glycosylation to glucose, xylose, hexose), for example, to increase plasma stability (notably, resistance towards exopeptidases).

The disclosed polypeptides or variants or fragments of reelin may include a deletion relative to full-length reelin (e.g., SEQ ID NO:1). The disclosed polypeptide fragments may include a deletion selected from an N-terminal deletion, a C-terminal deletion, and both, relative to full-length reelin. Further, in some embodiments the disclosed polypeptide fragments may include an internal deletion. The deletion may remove at least about 1, 2, 3, 4, 5, 10, 15, 20, 25, 50, 100, 150, 200 amino acids or more of full-length reelin.

Pharmaceutical Compositions

The compositions disclosed herein may include pharmaceutical compositions comprising a reelin polypeptide, variants and/or fragments thereof, and may be formulated for administration to a subject in need thereof. Compositions may include one, or more than one, different reelin polypeptide and/or variant(s) (e.g., a composition may include one or more of SEQ ID NO:1, SEQ ID NO: 1 and fragments thereof). Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, preservatives, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral or parenteral delivery (e.g., intraarterially, intravenously, intraperitoneally, subcutaneously, or intramuscularly). In some embodiments, the pharmaceutical compositions are administered via intracoronary, epicardial, or endocardial injection. In some embodiments, compositions are formulated for site-specific administration, such as topical administration. By way of example, but not by way of limitation, in some embodiments, compositions are formulated for administration via a collagen patch. The collagen patch can be placed in contact with the tissue to be treated (e.g., cardiac tissue), and the therapeutic composition is then released to the tissue. In other embodiments, the reelin protein or fragment thereof is administered to the subject, e.g., the subject's heart, via viral particles.

In some embodiments, compositions are formulated for delivery by viral particles. Their high stability, easily modifiable surface, and enormous diversity in shape and size, distinguish viruses from synthetic nanocarriers used for drug delivery. Several animal viruses are widely recognized as delivery vehicles or “vectors” for gene therapy (e.g., viral vectors for a nucleic acid that is capable of expressing a reelin polypeptide or variant thereof), and can also be employed as nanocarriers (e.g., carrying a reelin polypeptide or variant thereof). In addition, plant and bacterial viruses (e.g., phages) have been investigated and applied as drug carriers. The genetic material within the capsids or coat of viruses and phages can be removed to produce empty viral-like particles that are replication-deficient and can then be loaded with therapeutic agents, for example reelin polypeptide and/or variant(s) thereof. Exemplary viruses include, but are not limited to phages such as M13, T4, T7, MS2, and λ, the tobacco mosaic virus (TMV), cowpea chlorotic mottle virus (CCMV), and cowpea mosaic virus (CMV).

In some embodiments, compositions are formulated for delivery as nanoparticles. In one aspect, the present invention provides a nanoparticle-polypeptide complex comprising a bioactive polypeptide (e.g., a reelin polypeptide or variant thereof) in association with a nanoparticle. The nanoparticle can be a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene or a nanotube. In some embodiments, the nanoparticle is a lipid-based nanoparticle or a superparamagnetic nanoparticle. Non-limiting examples of lipid-based nanoparticles include liposomes and DOTAP:cholesterol vesicles. A nanoparticle-polypeptide complex can contain a second bioactive polypeptide in association with the nanoparticle, and/or one or more additional active agents.

The therapeutic composition may include, in addition to a reelin polypeptide, or variants thereof, one or more additional active agents. By way of example, the one or more active agents may include an antibiotic, anti-inflammatory agent, a steroid, or a non-steroidal anti-inflammatory drug.

According to various aspects, a reelin polypeptide, or variant thereof, and optionally the one or more active or inactive agents may be present in the composition as particles or may be soluble. By way of example, in some embodiments, micro particles or microspheres may be employed, and/or nanoparticles may also be employed, e.g., by utilizing biodegradable polymers and lipids to form liposomes, dendrimers, micelles, or nanowafers as carriers for targeted delivery of the reelin polypeptide or variant thereof. In some embodiments, polymeric implants may be used. By way of example, but not by way of limitation, in some embodiments, a therapeutic composition comprising a reelin polypeptide or variant thereof is applied to a collagen patch.

In some embodiments, the composition formulated for administration comprises between 0.1 ng and 500 mg/ml of the reelin peptide, or variant thereof. In some embodiments, the compositions if formulated such that between 0.1 ng and 500 μg of the reelin peptide, or variant thereof is administered to a subject. In some embodiments, the compositions if formulated such that between about 1 and 100 μg, between about 100 and 200 μg, between about 200 and 400 μg, between about 300 and 500 μg, between about 10 and 50 μg, or about 15-30 μg or about 20 μg of reelin protein is provided. In some embodiments, the composition is formulated such that between about 10 fmol and 500 pmol is administered to the subject.

Cardiac Diseases, Conditions, and Injuries

Provided herein are compositions and methods useful to treat disease, conditions, or injuries of the heart to a subject in need thereof. Subjects suitable for the disclosed methods of treatment may include, but are not limited to, subjects having or at risk for developing disease, conditions or injury that negatively affect the heart. By way of example, but not by way of limitation, in some embodiments, such subjects are suffering from, or at risk of one or more of (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) and endocarditis (bacterial, viral, or fungal).

In some embodiments, the heart disease, condition or injury result in one or more symptoms, including, but not limited to (1) arrhythmia; (2) tachycardia; (3) bradycardia; (4) chest pain (angina); (5) fainting; (6) swollen feet or ankles; (7) cyanosis; (8) dizziness; (9) decreased cardiac lymphatics; (10) cardiac fluid accumulation and/or cardiac inflammation; (11) decreased systolic function; (12) atherosclerotic plaque formation; (13) delayed cardiac healing process and cardiac repair; (14) adverse cardiac remodeling; (15) shortness of breath; (16) weakness or fatigue.

In some embodiments, the cardiac disease, condition, or injury includes myocardial infarction.

Causes of cardiac diseases, conditions, or injuries are not intended to be limiting and can include any one or more of the following: trauma, infections (bacterial, viral, fungal), sensitivity to non-infectious bacteria or toxins, allergies, transplant, cancer, exposure to toxins, genetic predisposition, congenital conditions, lifestyle choices, age.

Methods of diagnosing cardiac diseases, conditions, and injuries, and method for monitoring improvement in the symptoms of such disease, condition, and injuries (e.g., during and/or after treatment) include but are not limited to echocardiogram, transesophageal echocardiography (TEE), electrocardiogram (ECG or EKG), magnetic resonance imaging (MRI), CT scan, exercise/cardiac stress test, pharmacologic stress test, tilt test, ambulatory rhythm monitoring tests, coronary angiogram, physical examination including blood pressure, heart rate monitor, pulse oximeter, and patient interview.

Methods

Disclosed herein are methods of treating a cardiac condition that comprises administering to a patient in need thereof, a pharmaceutical composition comprising a reelin polypeptide, or a fragment or variant thereof. In some embodiments, the subject is diagnosed or is at risk of developing a disease or condition that negatively impacts the heart such as, but not limited to (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) endocarditis (bacterial, viral, or fungal); (18) cancer. In some embodiments, the subject exhibits one or more symptoms, which may include, but are not limited to (1) arrhythmia; (2) tachycardia; (3) bradycardia; (4) chest pain (angina); (5) fainting; (6) swollen feet or ankles; (7) cyanosis; (8) dizziness; (9) decreased cardiac lymphatics; (10) cardiac fluid accumulation and/or cardiac inflammation; (11) decreased systolic function; (12) atherosclerotic plaque formation; (13) delayed cardiac healing process and cardiac repair; (14) adverse cardiac remodeling; (15) shortness of breath; (16) weakness or fatigue.

In some embodiments, the composition is formulated for systemic delivery, and methods include administration via oral or parenteral delivery. In some embodiments, minimally invasive microneedles and/or iontophoresis may be used to administer the composition. In some embodiments, the composition is formulated for delivery via viral particles.

In some embodiments, the methods include administration a therapeutic composition comprising a reelin polypeptide, or variant or fragment thereof, to a subject by contacting the subject heart tissue with a collagen patch embedded with the therapeutic composition.

In some embodiments, the methods include administration of the therapeutic compositions once per day; in some embodiments, the composition may be administered multiple times per day, e.g., at a frequency of one or two times per day, or at a frequency of three or four times per day or more. In some embodiments, the methods include administration of the composition once per week, once per month, or as symptoms dictate. In

In some embodiments, the composition is administered such between about 0.1 ng and 500 mg/ml of the reelin peptide, or variant thereof reaches the heart of the subject. In some embodiments, the composition is administered such between about 0.1 ng and 500 μg of the reelin peptide, or variant thereof reaches the heart of a subject. In some embodiments, the compositions if formulated such that between about 1 and 100 μg, between about 100 and 200 μg, between about 200 and 400 μg, between about 300 and 500 μg, between about 10 and 50 μg, or about 15-30 μg or about 20 μg of reelin reaches the heart of the subject. In some embodiments, the composition is administered such between about 10 fmol and 500 pmol reaches the heart of the subject.

In some embodiments, the treatment reduces, alleviates, prevents, or otherwise lessens the symptoms of the disease or condition more quickly than if no treatment is provided to a subject suffering the same or similar disease, condition or injury.

In some embodiments, improvements in the condition of the subject's heart is observed more quickly than if no treatment is provided for the same or similar condition or disease.

By way of example, in some embodiments, improvements in the condition of the subject's heart is observed within about 6 hours, within about 12 hours, or within about 24 hours of administration of the composition. In some embodiments, improvements in the condition of the subject's heart is observed within about 1 to about 3 days; within about 3 to about 5 days, or within about a week of the first administration. In some embodiments, improvements in the condition of the subject's heart is observed within about 10 days, about 14 days or within about 1 month of the first administration.

### EXAMPLES

The following examples are illustrative and should not be interpreted to limit the scope of the claimed subject matter.

### Example 1. Lymphoangiocrine Signals Promote Cardiac Growth and Repair

### Abstract

Recent studies suggested a beneficial role of lymphatics in restoring heart function after cardiac injury¹⁻⁶. The inventors report that in mice lymphatics promote cardiac growth, repair and protection. The inventors show that a lymphoangiocrine signal produced by lymphatic endothelial cells (LECs) controls cardiomyocyte (CM) proliferation and survival during heart development, improves neonatal cardiac regeneration and is cardioprotective after myocardial infarction (MI). Mutant embryos devoid of LECs develop smaller hearts consequence of reduced CM proliferation and increased CM apoptosis. Culturing primary mouse CMs in LEC-conditioned media increases CM proliferation and survival, indicating that LECs produce lymphoangiocrine signals controlling CM homeostasis. Characterization of the LEC secretome identified Reelin as a key player responsible for such function. Moreover, the inventors report that LEC-specific Reln-null embryos also develop smaller hearts, that Reelin is required for efficient heart repair and function following neonatal MI, and that cardiac delivery of REELIN using collagen patches improves adult heart function after MI through a cardioprotective effect. These results identify a novel lymphoangiocrine role of LECs during cardiac development and injury response, and Reelin as an important mediator of this function.

Results

The molecular and functional characterization of the lymphatic vasculature has greatly improved¹. Recent data suggest that natural or therapeutic formation of new lymphatics (lymphangiogenesis) correlates with improved systolic function after experimental MI; it delays atherosclerotic plaque formation, facilitates the healing process after MI, and can be a natural response to fluid accumulation into the myocardium during cardiac edema^{2,3,4}. These new findings argue that stimulation of lymphangiogenesis in the infarcted heart could improve cardiac function and prevent adverse cardiac remodeling³.

Studies in mouse and zebrafish suggested that newly formed lymphatics provided a route for the clearance of immune cells in the injured heart, and therefore promote cardiac repair^{5,6}. However, whether lymphatics have additional functional roles during heart development and cardiac repair is not known.

Lymphatics Regulate Heart Growth

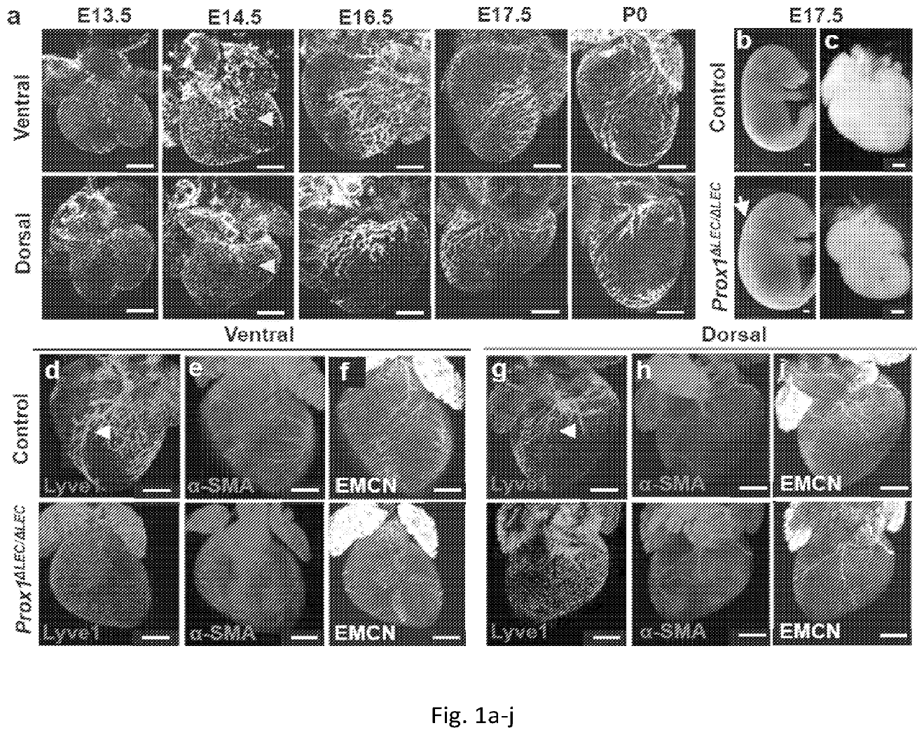

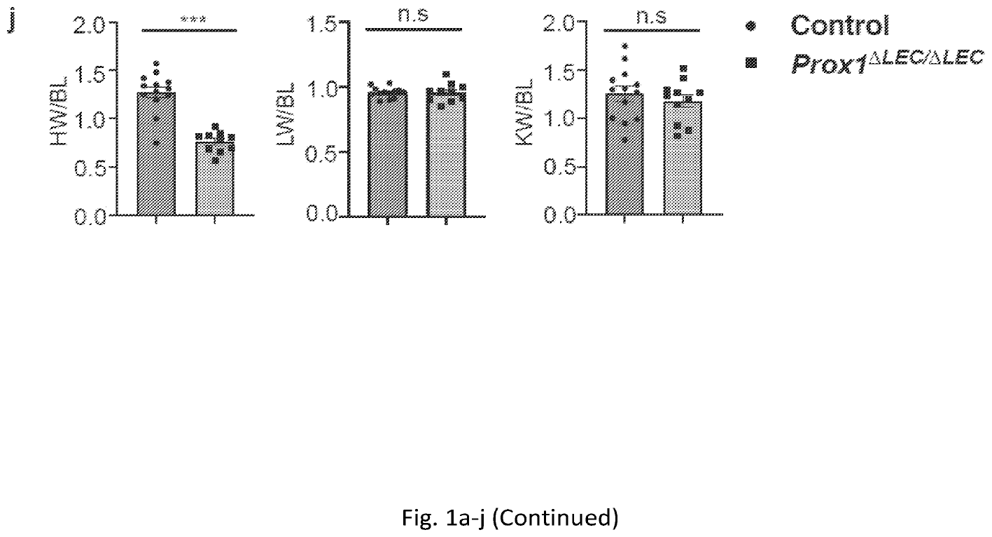

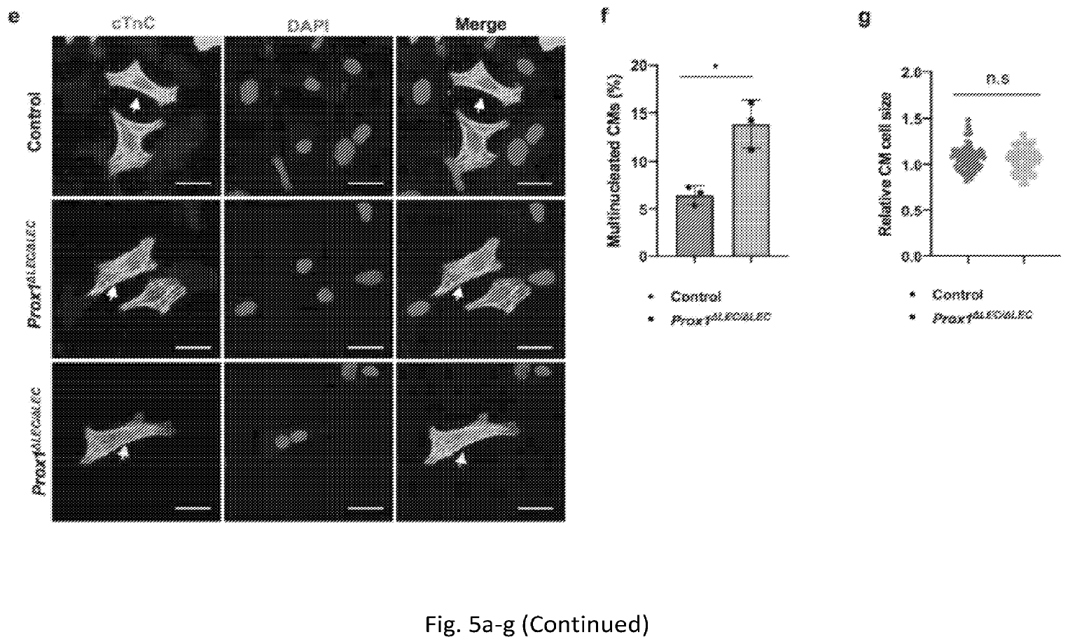

As previously reported², at around E14.5 cardiac lymphatics become evident, particularly over the dorsal side of the heart (FIG. 1a). As development progresses, lymphatics expand over the dorsal and ventral surfaces, and into the myocardium during embryonic and postnatal stages (FIG. 1a). To evaluate a possible developmental role of cardiac associated lymphatics, the inventors took advantage of Prox1 floxed mice⁷. Prox1 is a master regulator required to promote and maintain LEC fate identity^{8,9} and germ-line deletion of Prox1 in mice results in complete lack of LECs and embryonic lethality at around E14.5⁸. To conditionally delete Prox1 from LECs, Cad5(PAC)-CreER^{T2} mice¹⁰ were crossed with Prox1 floxed mice and injected pregnant females with tamoxifen at E13.5 and E14.5. Analysis of E17.5 Cad5(PAC)-CreER^{T2}; Prox1^{f/f} null embryos (Prox1^{ΔLEC/ΔLEC}) revealed appreciably edema (FIG. 1b, arrow); a phenotype associated with defective lymphatics and they die soon after birth. Surprisingly, these mutant embryos have significantly smaller hearts than control littermates (approximately ⅓ smaller, FIG. 1c, j). Most, if not all cardiac lymphatics were missing (FIG. 1d, g and FIG. 5a) and the blood vasculature was not affected in Prox1^{ΔLEC/ΔLEC} embryos (FIG. 1e, f, h, i).

Decreased CM Mass Causes Heart Size Reduction

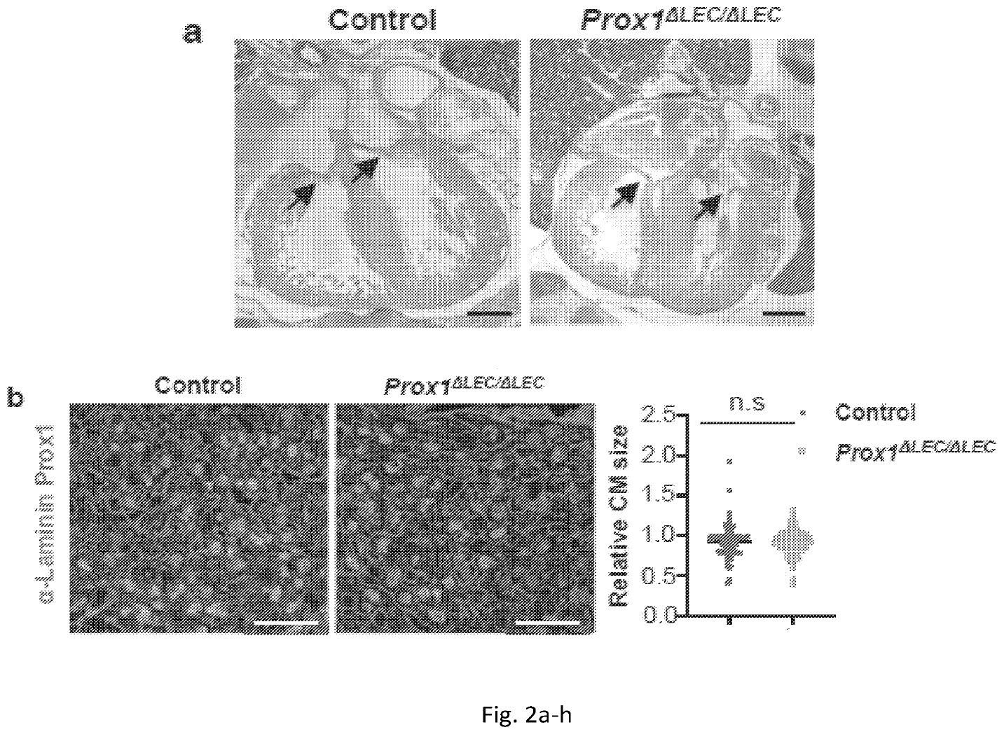

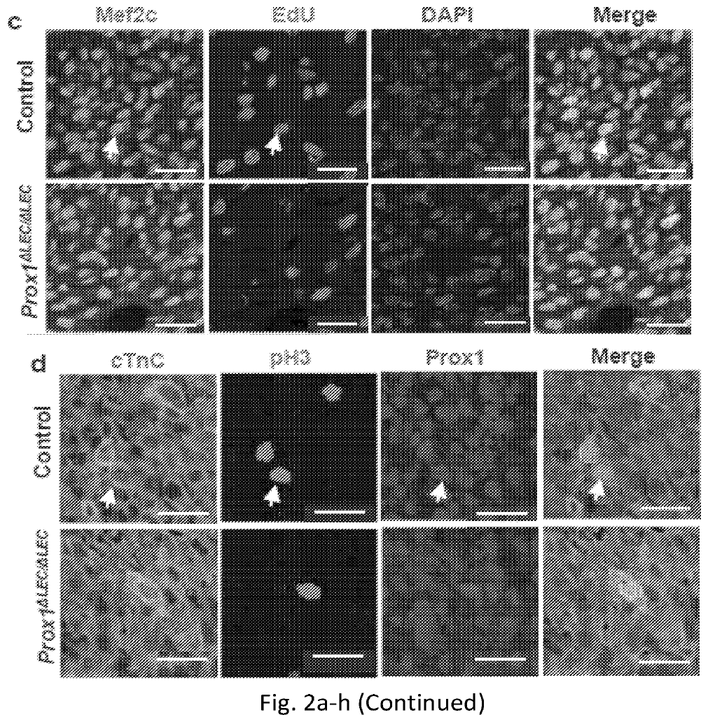

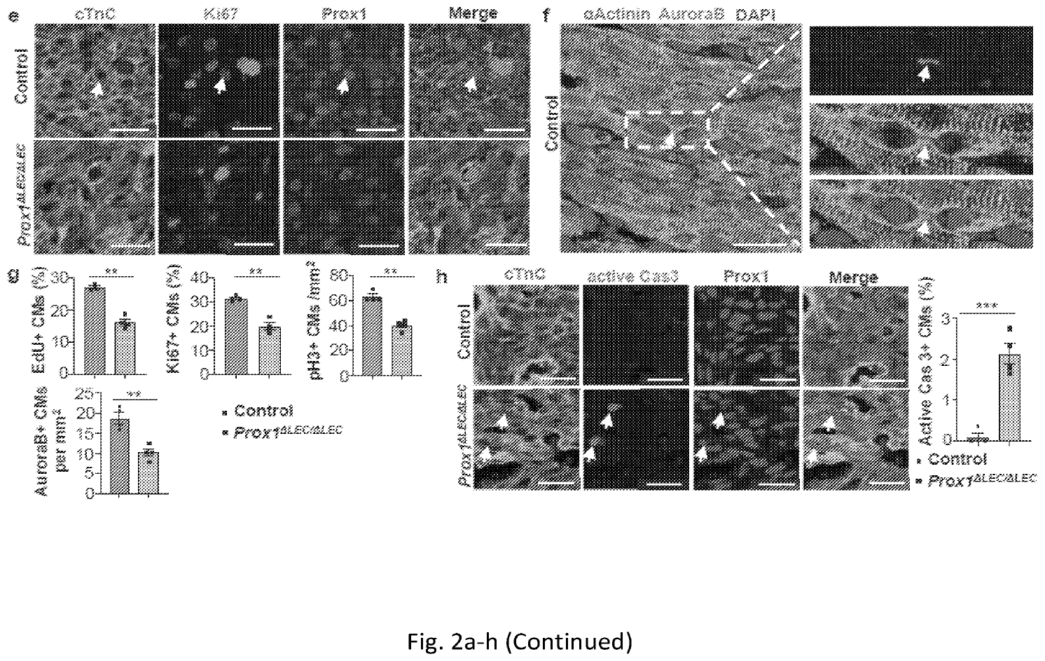

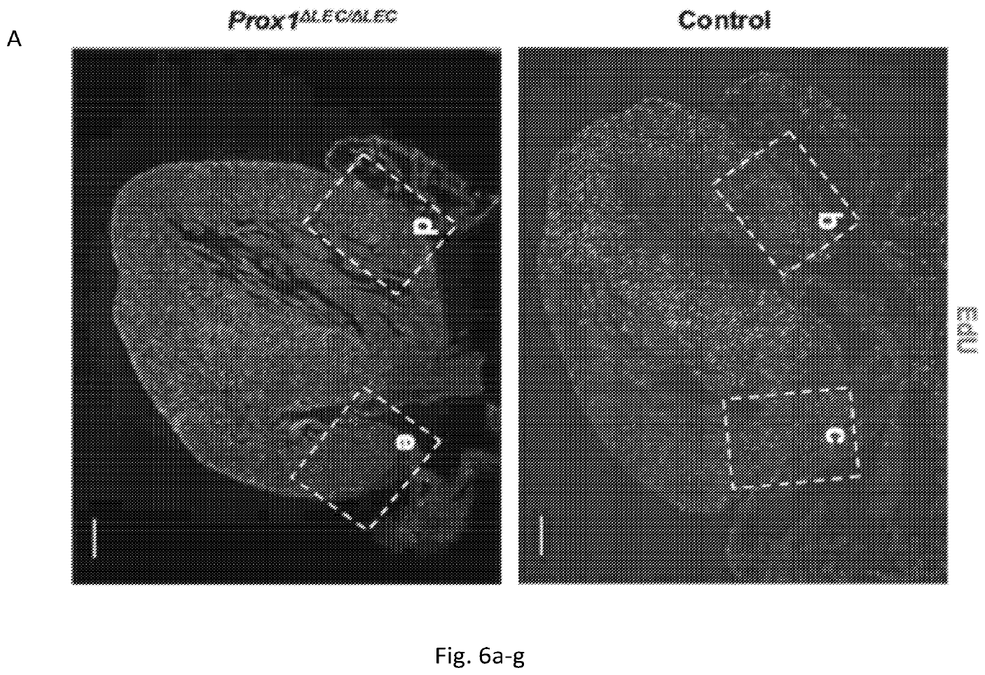

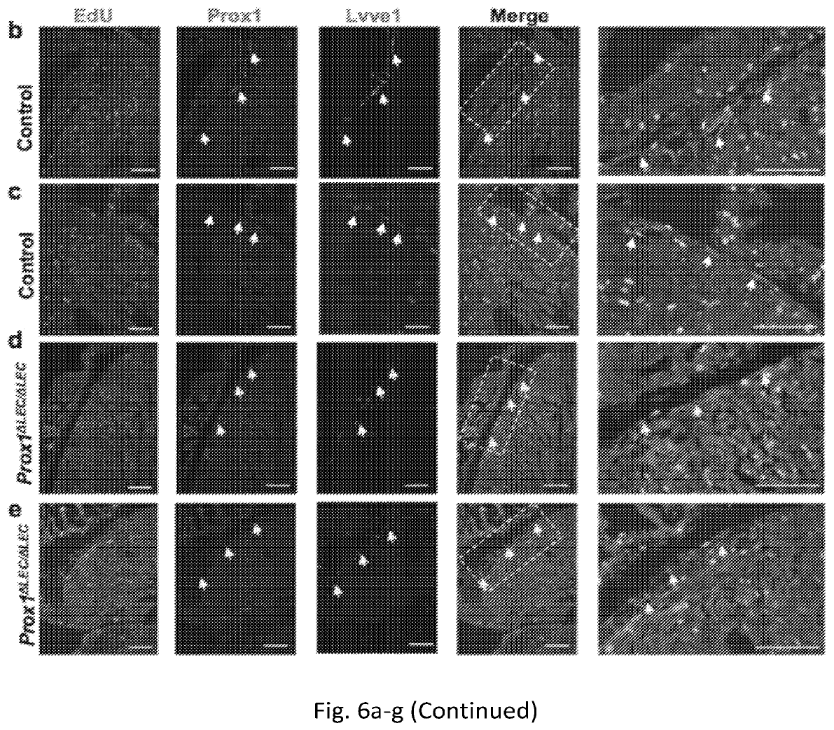

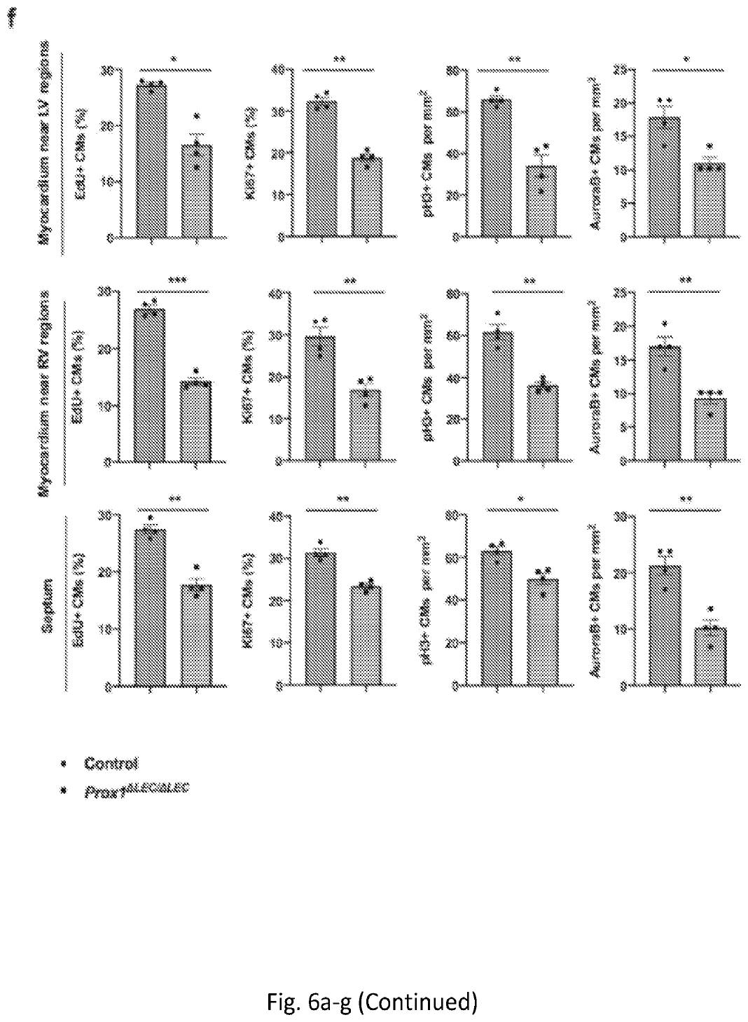

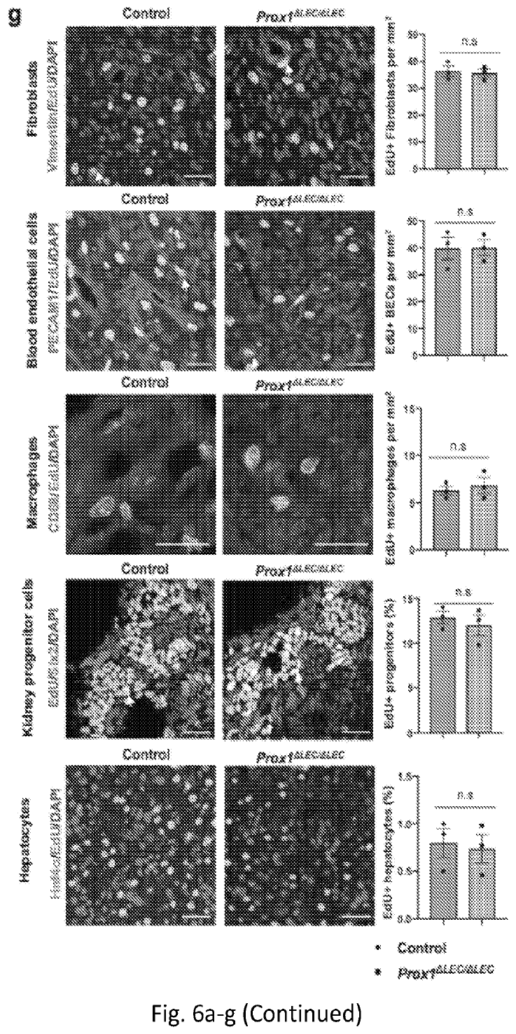

H&E staining confirmed that the overall size of the ventricles in Prox1^{ΔLEC/ΔLEC} embryos is smaller; however, cardiac valves appear normal (FIG. 2a, arrows). Immunostaining of heart sections against α-actinin and F-actin show that overall, cardiac muscle structure and arrangement are not disrupted in Prox1^{ΔLEC/ΔLEC} hearts (FIG. 5b). Flow cytometry analysis (FACS) indicated that the percentage of CMs is significantly reduced (approximately ⅓ reduction) (FIG. 5c), a result suggesting that a decrease in CM mass underlies the reduction in heart size. Hoechst 33342 labeling showed no differences in CMs ploidy in Prox1^{ΔLEC/ΔLEC} hearts (FIG. 5d). However, an increased percentage of multinucleated CMs was observed in these E17.5 mutant hearts after CM dissociation and o/n plating (FIG. 5e, f), but no overall differences in CM size were detected (FIG. 5e, g). Similarly, α-Laminin staining showed that the overall CM size was not affected in the mutant hearts (FIG. 2b). Next, the inventors evaluated possible alterations in CM proliferation and survival. Indeed, CM proliferation is greatly reduced in E17.5 Prox1^{ΔLEC/ΔLEC} embryos, as indicated by EdU labeling (FIG. 2c, g, FIG. 6a-e), and phospho-histone H3 (pH3), Ki67 and aurora kinase B (AuroraB) immunostainings (FIG. 2d-g). This reduction in proliferation is seen in different regions of the E17.5 mutant heart (FIG. 6f). In addition, CM apoptosis was significantly increased in Prox1^{ΔLEC/ΔLEC} hearts (FIG. 2h). These alterations in CM proliferation and apoptosis were not seen in other cardiac cell types (blood endothelial cells, fibroblasts or macrophages) or in other organs (nephron progenitors and hepatocytes) in these mutant embryos (FIG. 6g).

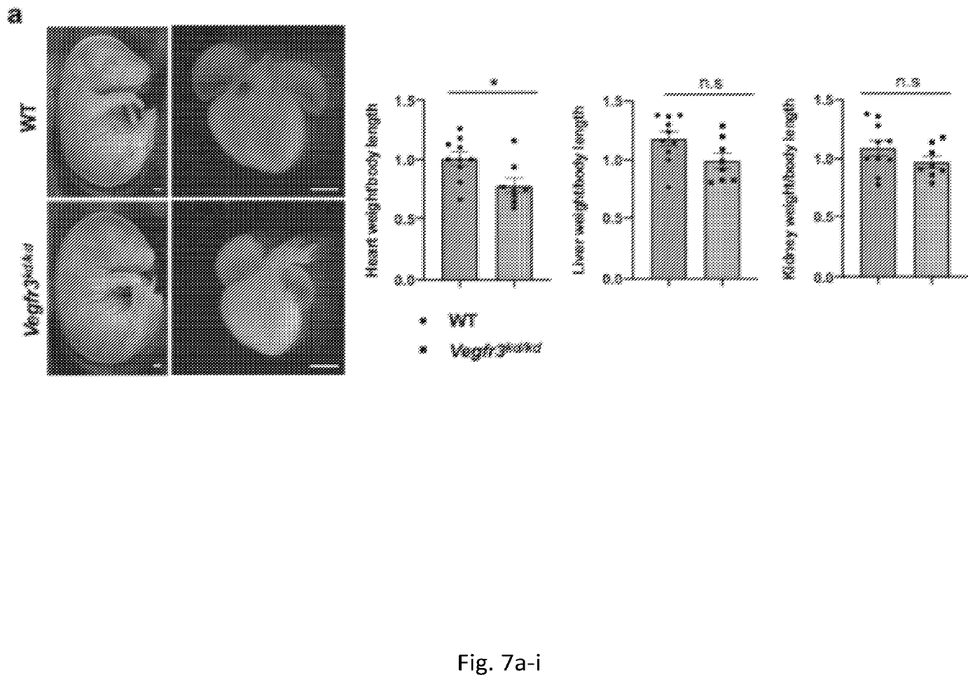

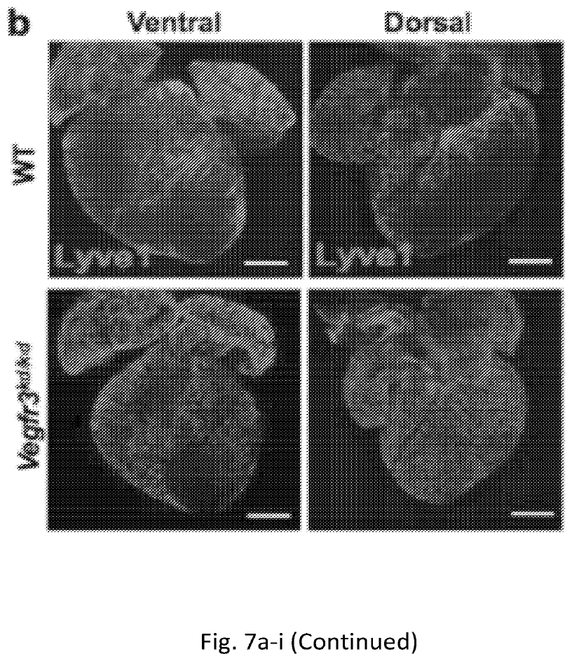

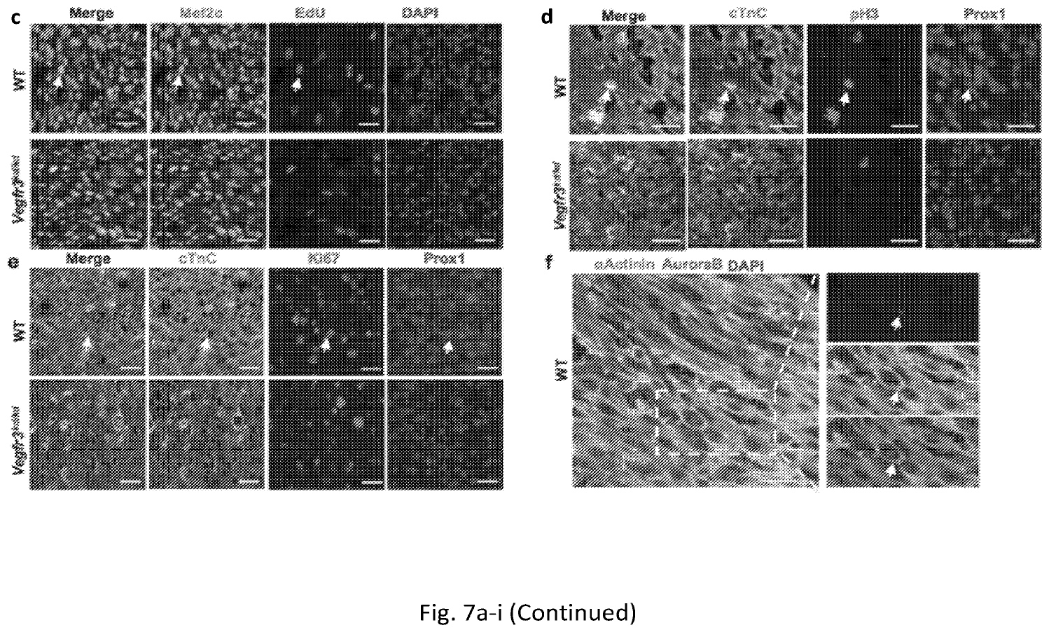

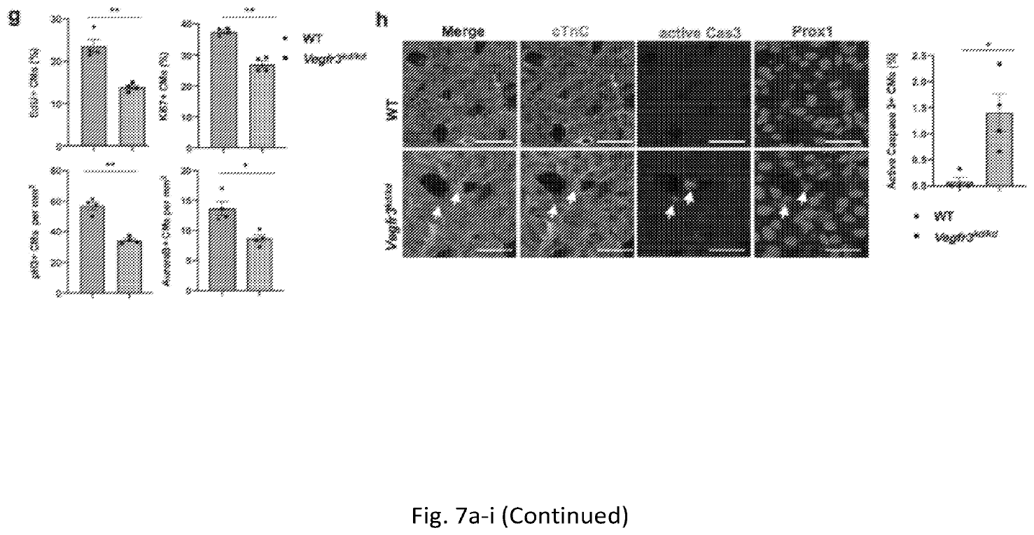

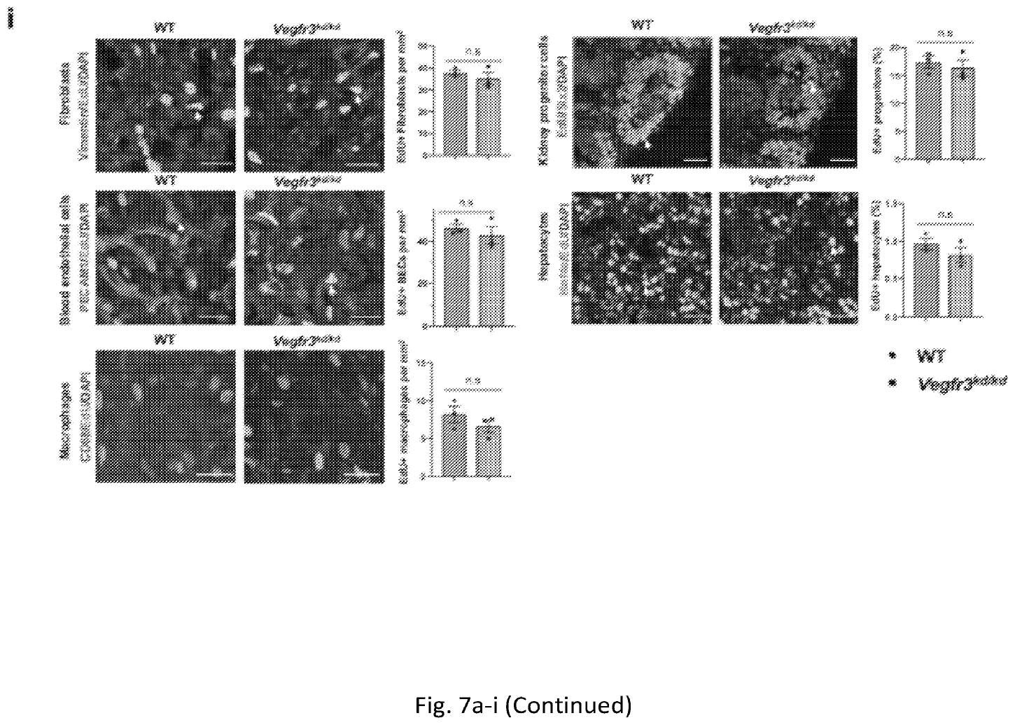

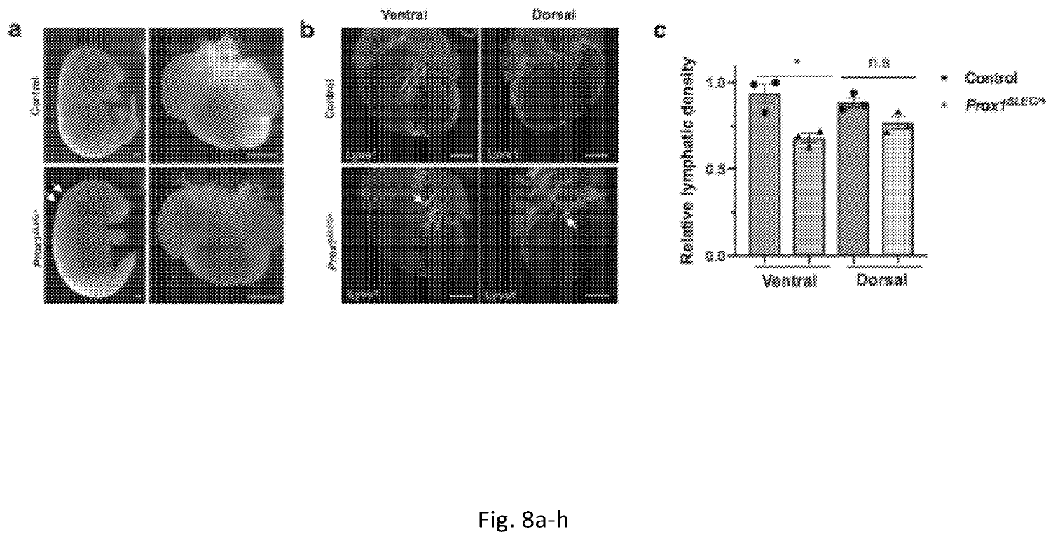

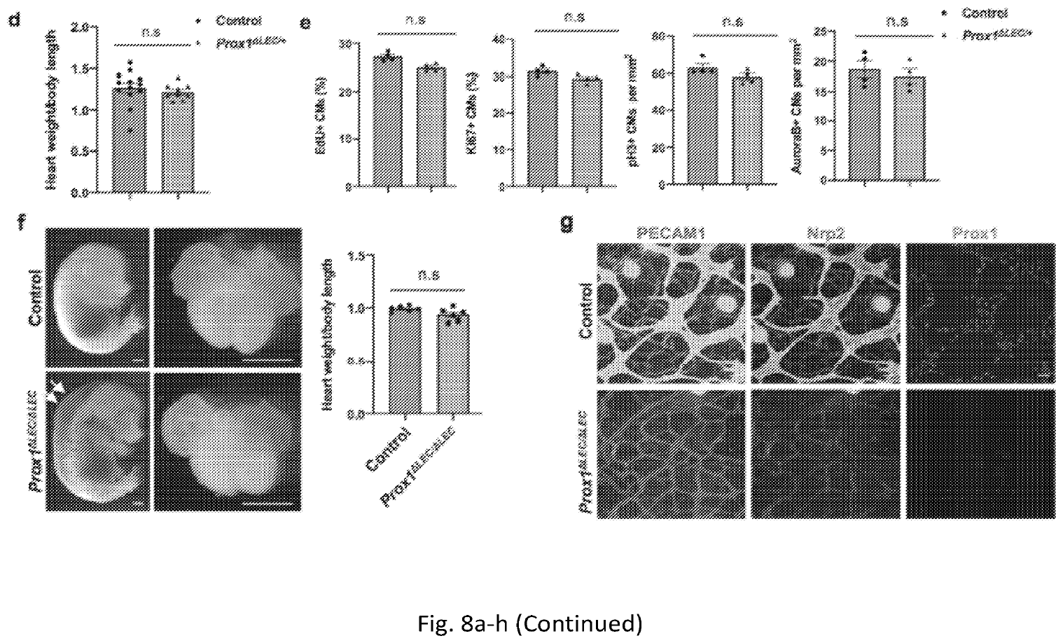

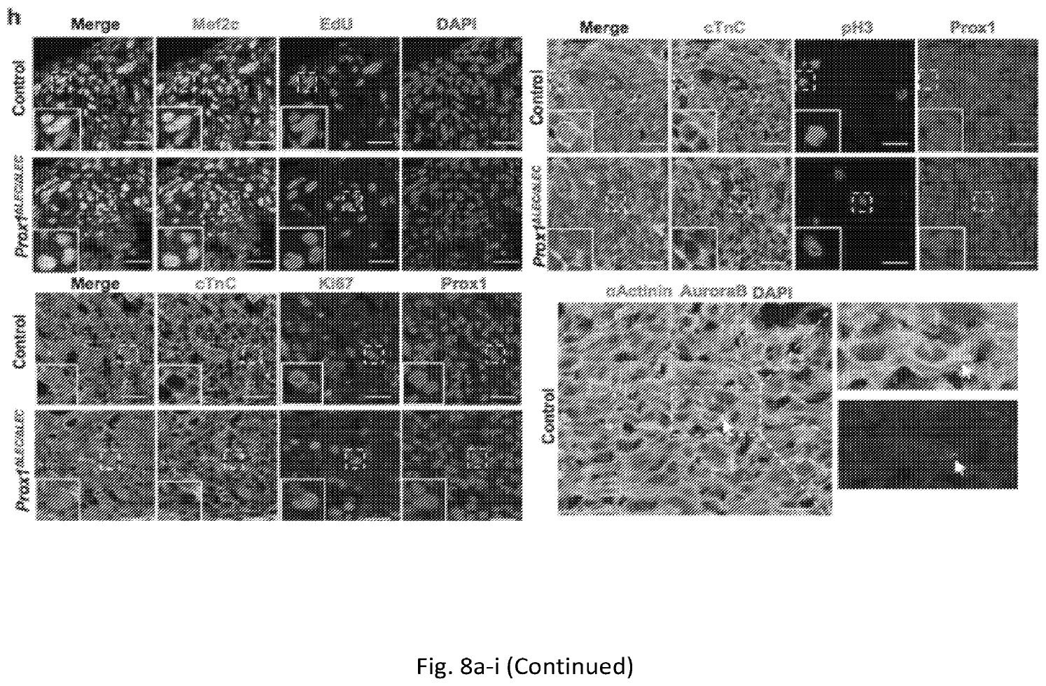

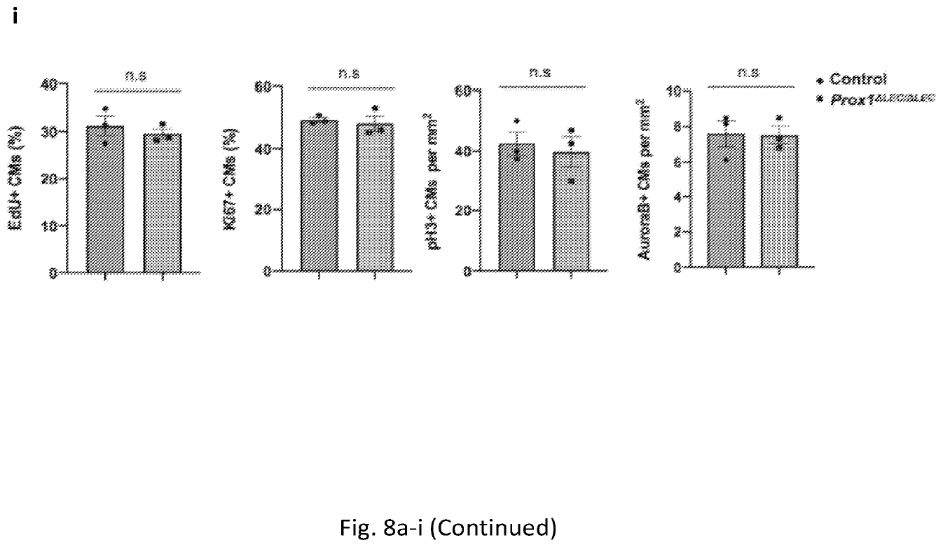

To support these findings, the inventors performed similar analysis using another mouse model without lymphatics. Accordingly, the inventors used Vegfr3^{kd/kd}, a natural occurring mouse strain with a point mutation in the kinase domain of VEGFR3 that impacts Vegfr3 signaling and therefore, lymphatic development¹¹. As seen in FIG. 7a, b, E17.5 Vegfr3^{kd/kd} embryos lacking cardiac-associated lymphatics also have smaller hearts. Similar to Prox1^{ΔLEC/ΔLEC} embryos (FIG. 1j), although no significant size differences were seen in E17.5 Vegfr3^{kd/kd} livers or kidneys, some individual samples show a size reduction trend (FIG. 7a). Also, similar to Prox1^{ΔLEC/ΔLEC} embryos, CM proliferation is significantly reduced in E17.5 Vegfr3^{kd/kd} hearts (FIG. 7c, g), CM apoptosis is significantly increased (FIG. 7h), and proliferation in other cardiac cell types or in nephron progenitors and hepatocytes is not affected (FIG. 7i). Because E17.5 Prox1^{ΔLEC/ΔLEC} embryos develop edema, their reduced heart size could be secondary to hemodynamic defects consequence of their lack of lymphatics and therefore, of lymphatic flow. However, E17.5 Cad5(PAC)-CreER^{T2}; Prox1^{f/+} embryos (Prox1^{ΔLEC/+}) also exhibit severe edema, their cardiac lymphatics show reduced branching, but their heart size and CM proliferation is normal (FIG. 8a-e). Similarly, E14.5 Prox1^{ΔLEC/ΔLEC} null embryos (a stage when cardiac lymphatics will just start to grow into the heart, FIG. 1a; tamoxifen injection at E10.5 and E11.5) also lack LECs and exhibit severe edema, but their heart size and CM proliferation are normal (FIG. 8f-h).

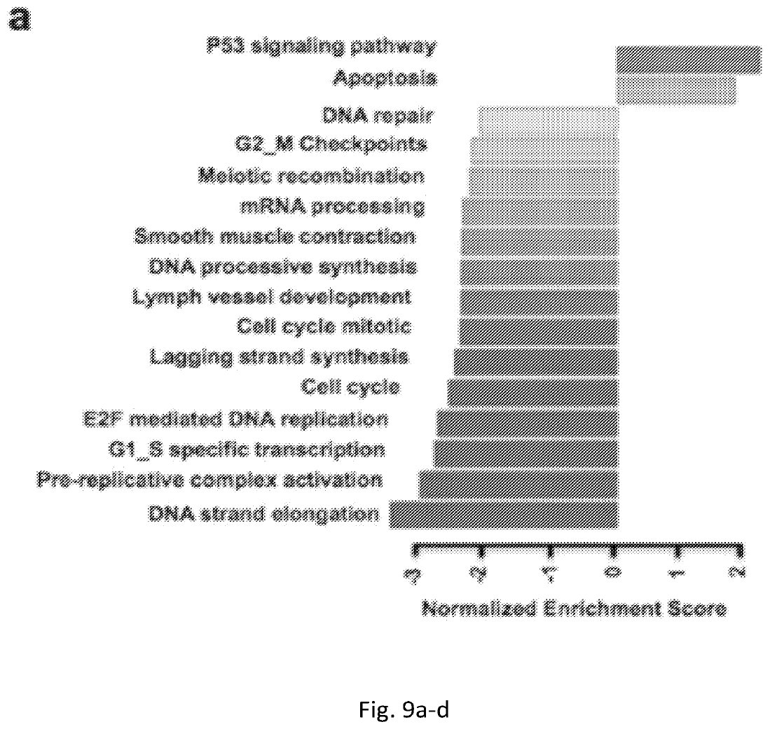

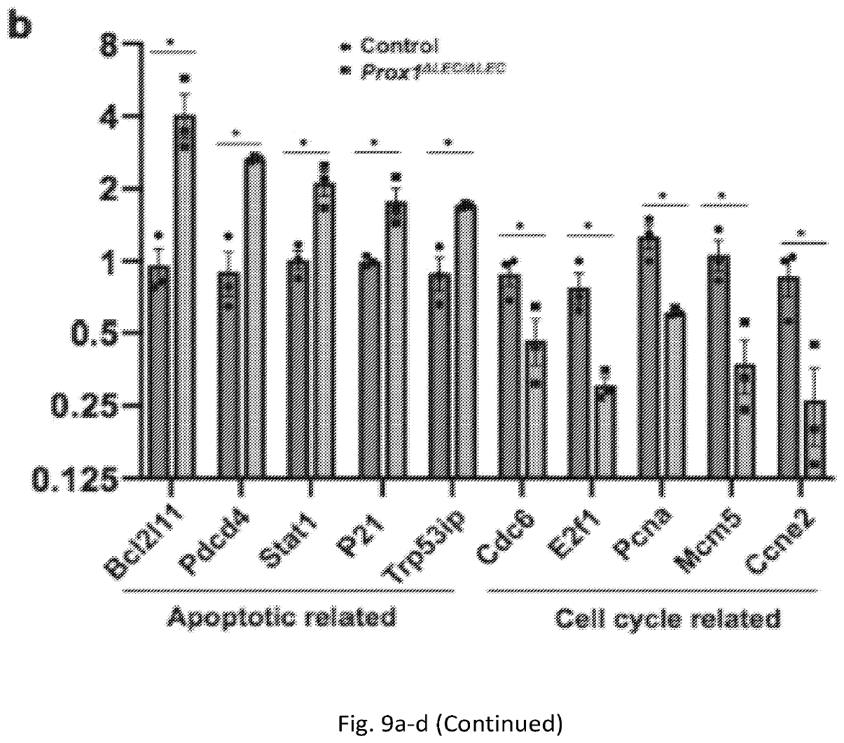

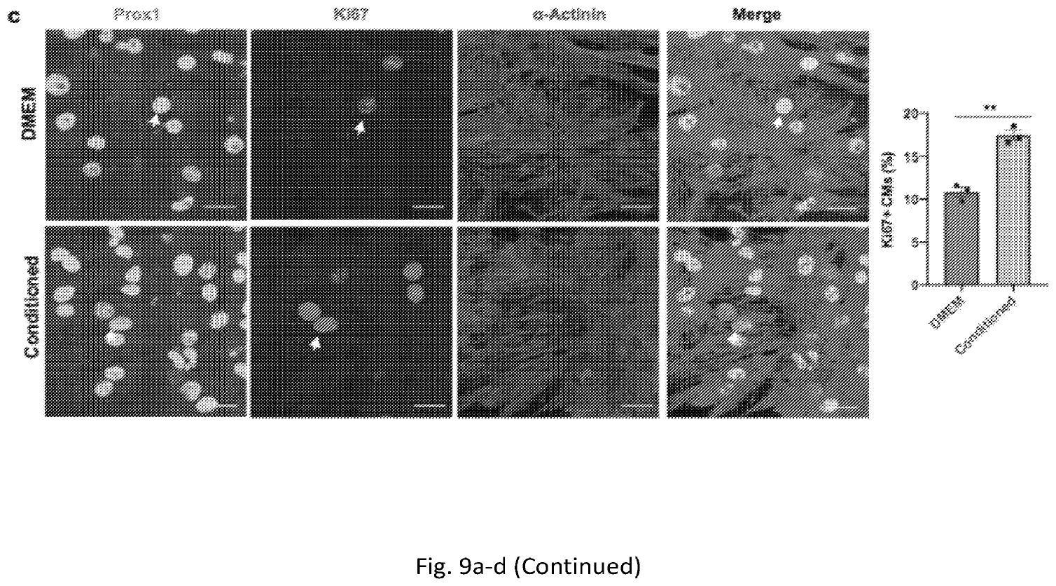

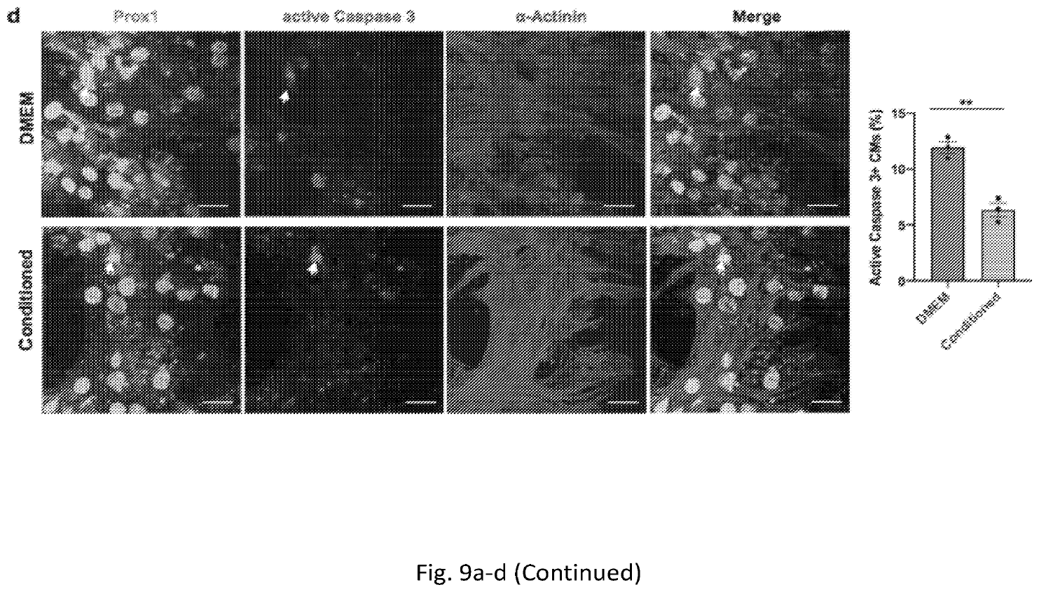

To investigate the molecular basis of this lymphatics-dependent defects, the inventors performed RNA sequencing (RNA-seq) of the ventricular portions of E17.5 control and Prox1^{ΔLEC/ΔLEC} hearts. Gene set expression analysis (GSEA) revealed that genes and pathways related to cell cycle were greatly reduced; instead, expression of genes and pathways involved in apoptosis were enriched (FIG. 5a). These results were validated by qPCR showing that expression of pro-apoptotic genes is significantly upregulated, but that of cell cycle related genes is significantly downregulated (Extended Data FIG. 9b).

LEC Media Promotes CM Proliferation

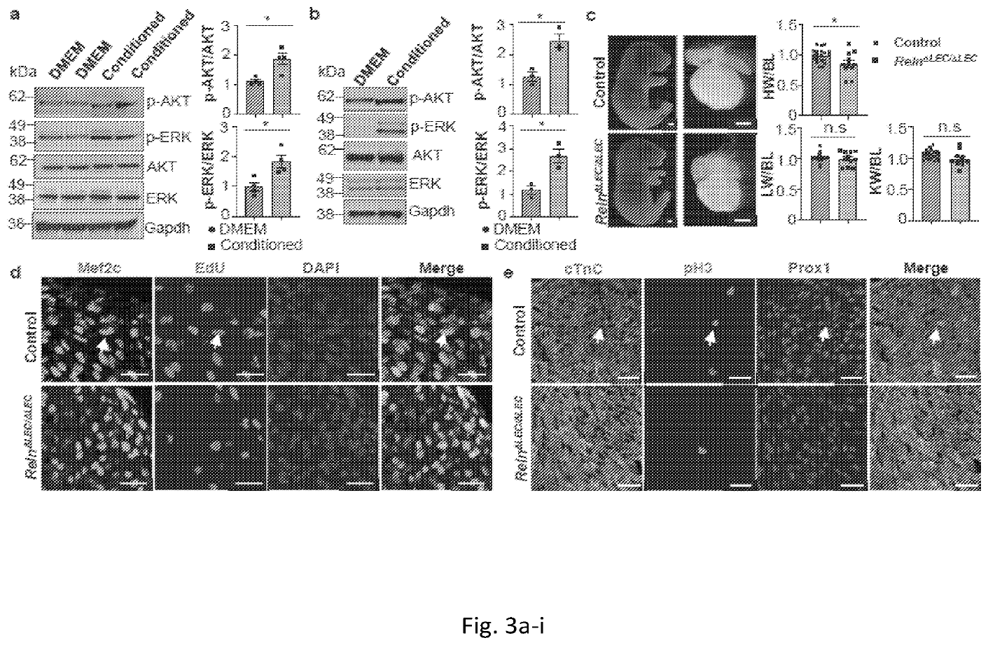

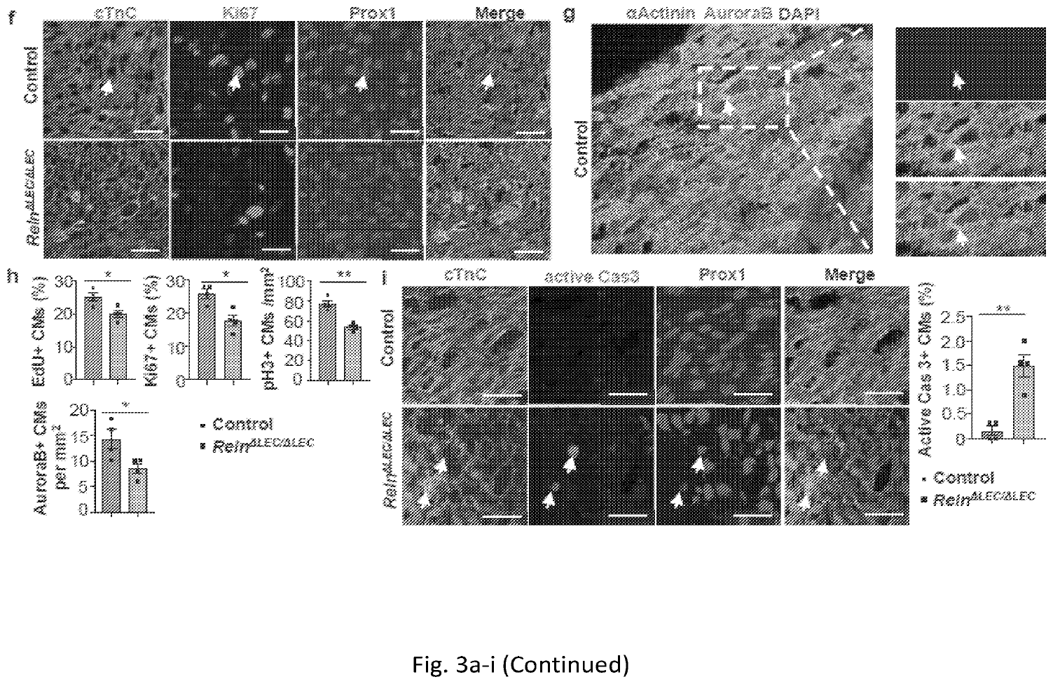

Signaling between blood endothelial cells (BECs) and CMs is important during cardiac growth and repair^{12,13}. To evaluate whether LECs produce lymphoangiocrine signals promoting CM proliferation and survival, the inventors first cultured human iPSCs-derived CMs (hiPSC-CMs) with LECs-conditioned media obtained from culturing commercially available human dermal LECs. AKT and ERK signaling was then examined since phosphorylated AKT and ERK (p-AKT and p-ERK) are frequently used as readouts of proliferative signaling. As seen in FIG. 3a, compared with DMEM control media, LEC-conditioned media significantly increases p-AKT and p-ERK signaling in the cultured hiPSC-CMs. Similar results were seen using mouse primary CMs isolated from wild-type E14.5 to E17.5 hearts (FIG. 3b). Furthermore, treatment of mouse primary CMs with LEC-conditioned media significantly increases cell proliferation as indicated by Ki67 staining (FIG. 9c), and protects CM from apoptosis when cultured under CoCl₂-induced hypoxia conditions (FIG. 9d). Together, these results argue that LECs-conditioned medium promotes CM proliferation and survival in vitro, and that lymphangiocrine factor/s present in that conditioned media play an important in vivo role during heart development.

Reelin is Required for Heart Growth

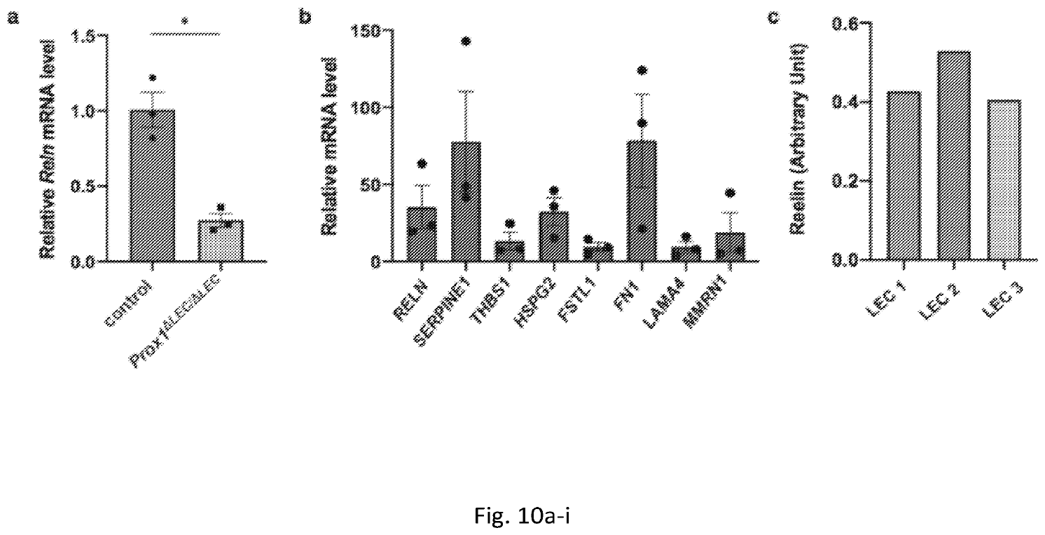

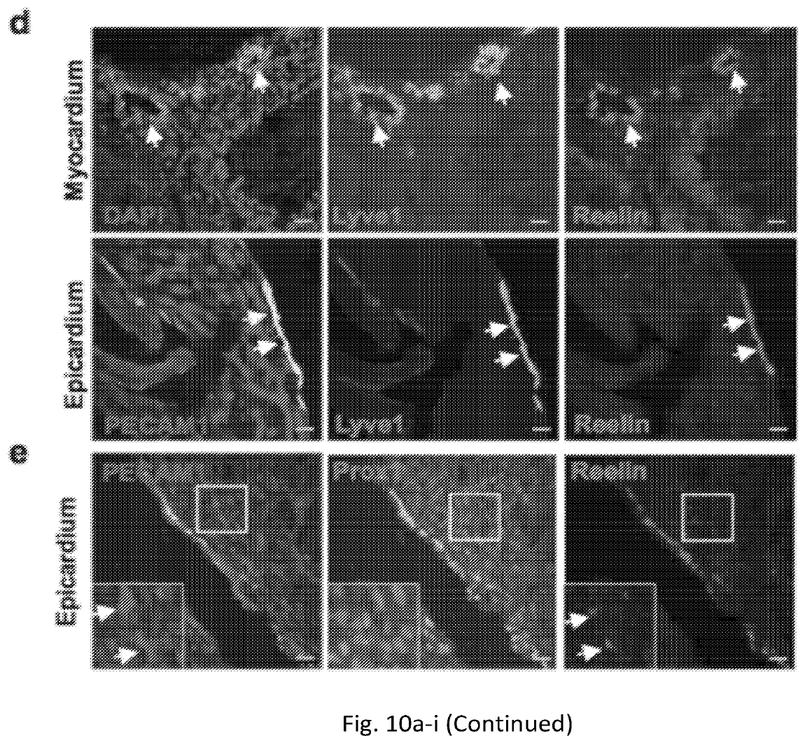

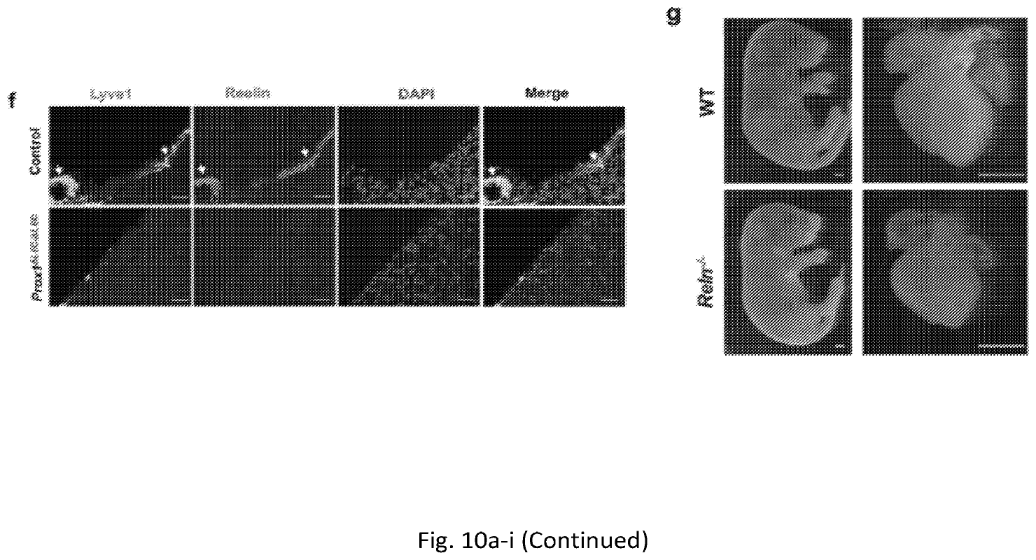

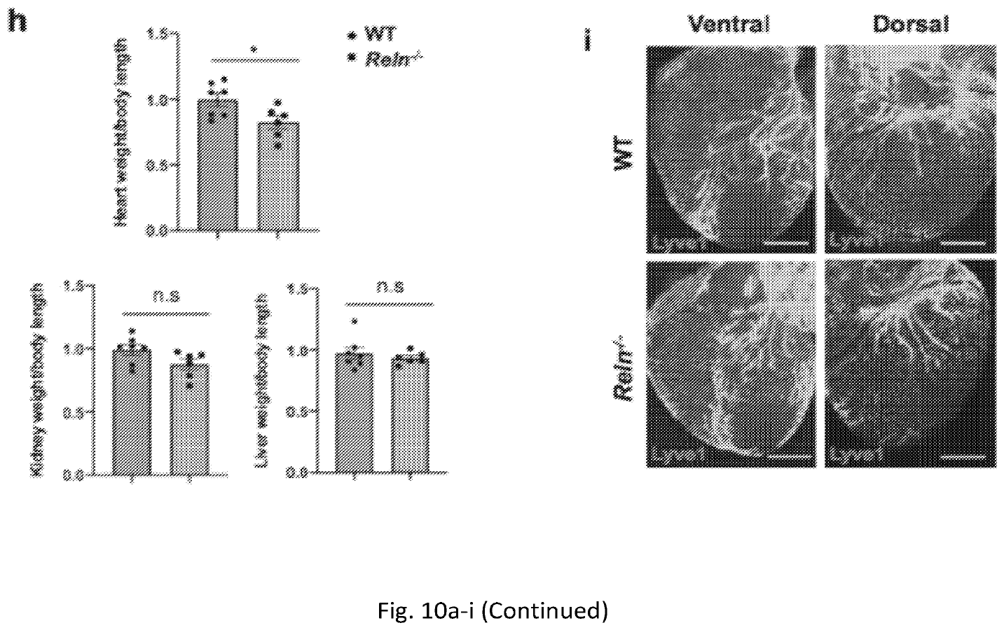

To identify such secreted factor/s the inventors performed mass spectrometry of the LEC conditioned media and identified 317 unique proteins. From that list, the inventors initially focused on all secreted proteins by comparing changes in their expression levels in the RNAseq dataset described above. Among those candidates, Reelin is greatly reduced in Prox1^{ΔLEC/ΔLEC} hearts (log 2 fold change: −0.6098 compared to control). Indeed, qPCR analysis confirmed about 80% reduction in Reln expression in Prox1^{ΔLEC/ΔLEC} hearts (FIG. 10a). The inventors then validated by qPCR the gene expression levels of Reln, as well as of several other enriched proteins identified in the LECs secretome (FIG. 10b). Quantification of Reelin secretion in 3 separate LEC preparations by ELISA revealed similar concentrations of this protein in their supernatants (average OD450 is 0.453±0.065) (FIG. 10c). Reelin is an extracellular matrix protein widely known for its roles during neuronal development and migration, and Reln mutant mice are ataxic^{14,15}. Reelin is also expressed in LECs and regulates collecting lymphatic vessel maturation¹⁶. In agreement with those results¹⁶, in the heart Reelin is mainly expressed in LECs (FIG. 10d), although some cardiac blood vessels also express low levels of Reelin (FIG. 10e). Accordingly, the observed qPCR reduction in Reln expression in Prox1^{ΔLEC/ΔLEC} hearts is consequence of their lack of lymphatics. Indeed, Reelin is almost undetected in E17.5 Prox1^{ΔLEC/ΔLEC} hearts (FIG. 10f).

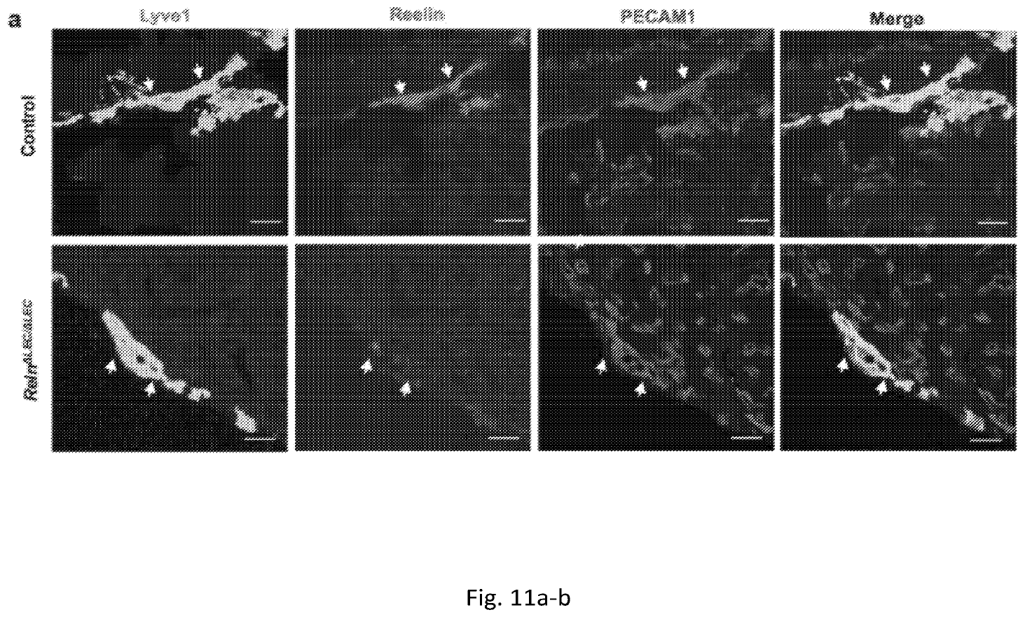

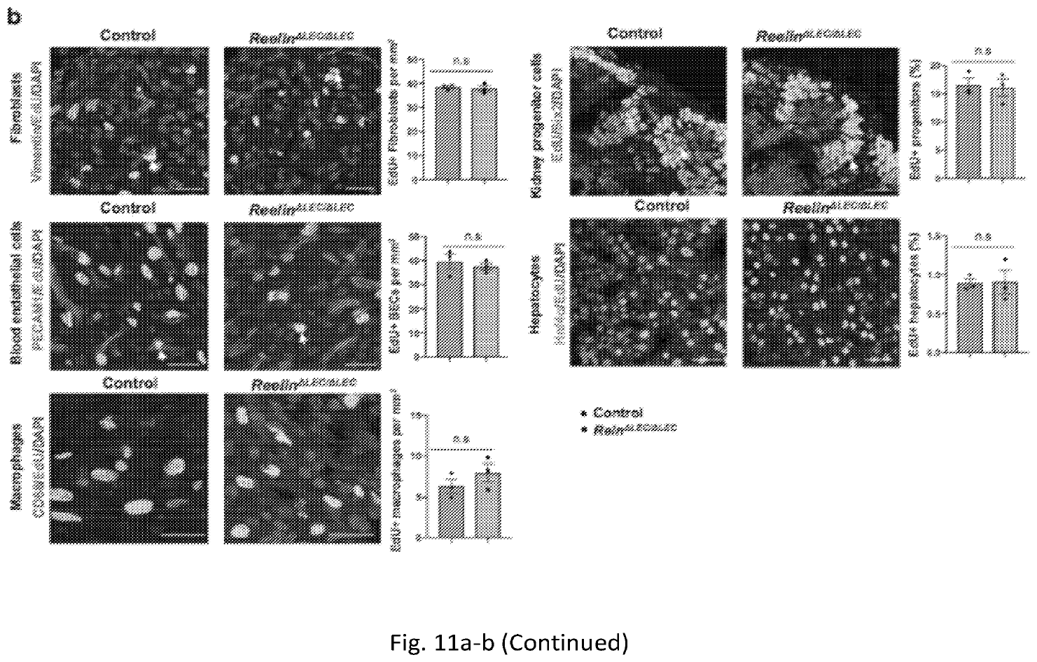

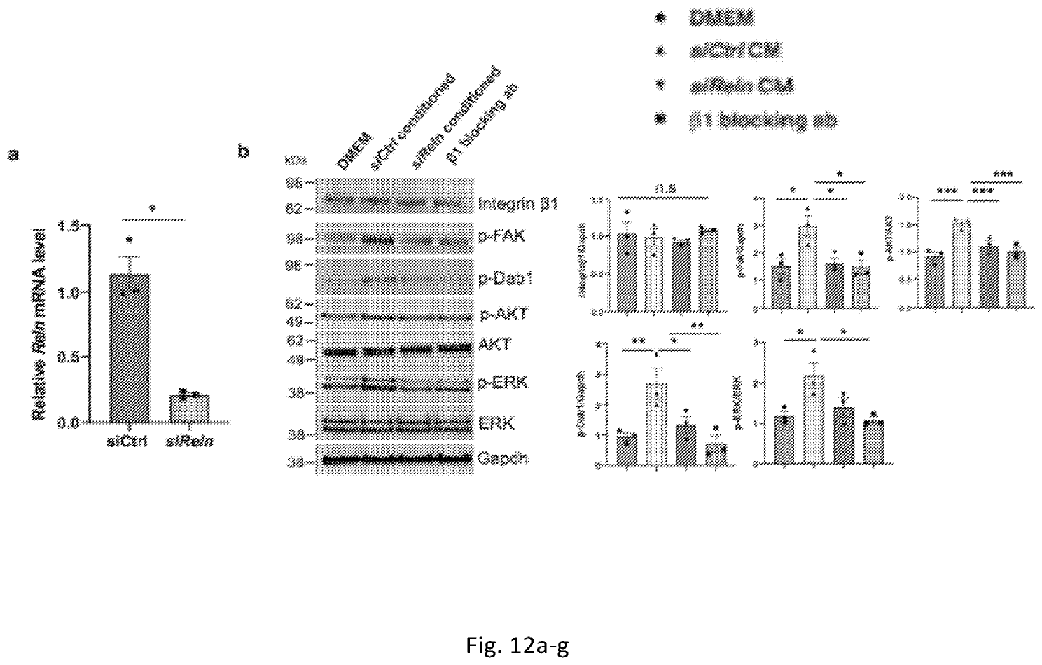

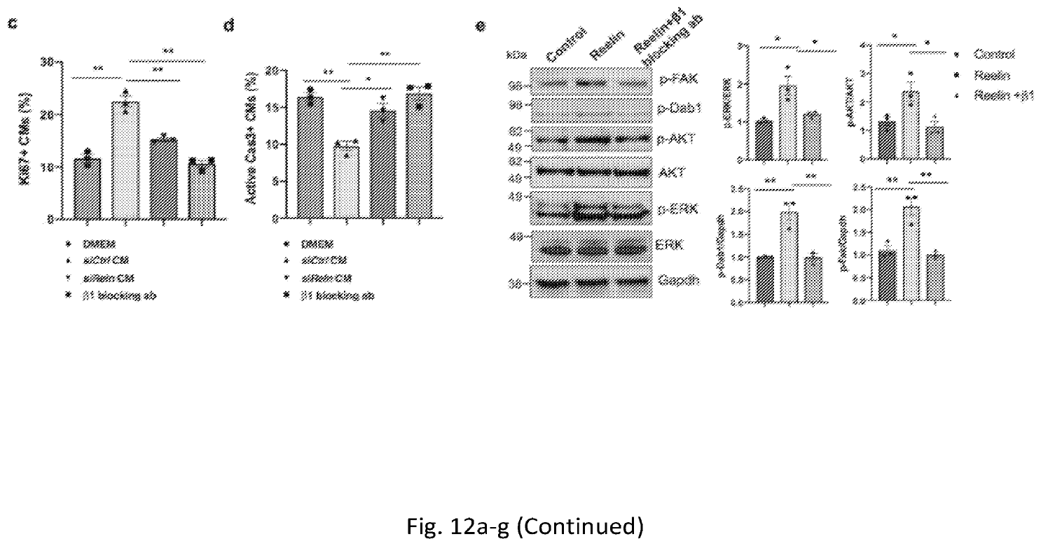

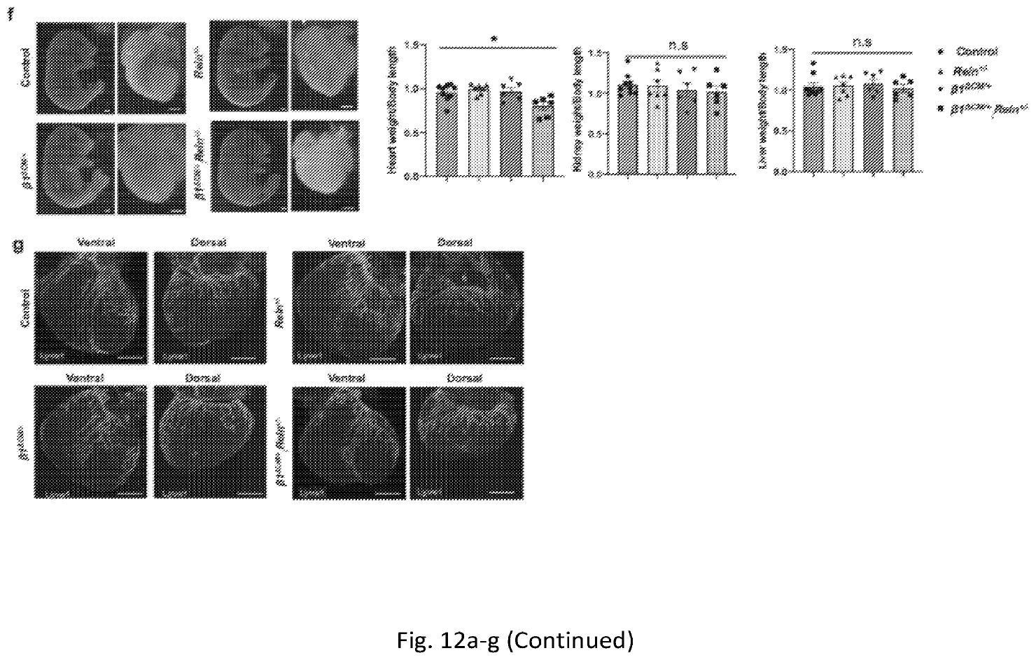

Importantly, the heart size of E17.5 Reln^{-/-} embryos¹⁷ was also significantly reduced, but cardiac lymphatics appear normal (FIG. 10g-i). To further demonstrate that the smaller heart phenotype is a consequence of Reln loss in LECs, we deleted Reelin from LECs (Reln^{ΔLEC/ΔLEC}) by crossing Reln floxed mice¹⁸ with Prox1CreER^{T2} mice¹⁹ (tamoxifen injections at E13.5 and E14.5). Immunostaining confirmed efficient Reelin deletion in cardiac lymphatics at E17.5 (FIG. 11a). Significantly, E17.5 Reln^{ΔLEC/ΔLEC} embryos also developed smaller hearts (although no significant differences are seen in the size of other organs such as kidneys and livers) (FIG. 3c). In addition, CM proliferation was also reduced in Reln^{ΔLEC/ΔLEC} embryos as indicated by EdU, pH3, Ki67 and AuroraB labeling (FIG. 3d-h), and CM apoptosis was increased as indicated by active Caspase 3 (FIG. 3i). No changes in proliferation and apoptosis were detected in other cardiac cell types or in kidney and liver (FIG. 11b). These results agree with those seen in Reln-i embryos, arguing that LEC-derived Reelin plays a critical role during heart development and growth by regulating CM proliferation and apoptosis. To validate this statement, the inventors collected LEC-conditioned media from Reln siRNA and control siRNA treated LECs. Analysis by qPCR showed that Reln expression is efficiently silenced in Reln siRNA treated LECs (FIG. 12a). Western analysis showed that the identified increase in p-AKT and p-ERK signaling induced by the LEC conditioned media was greatly reduced when using the Reln deficient LEC conditioned media (FIG. 12b).

Reelin Signaling Requires Integrinβ1

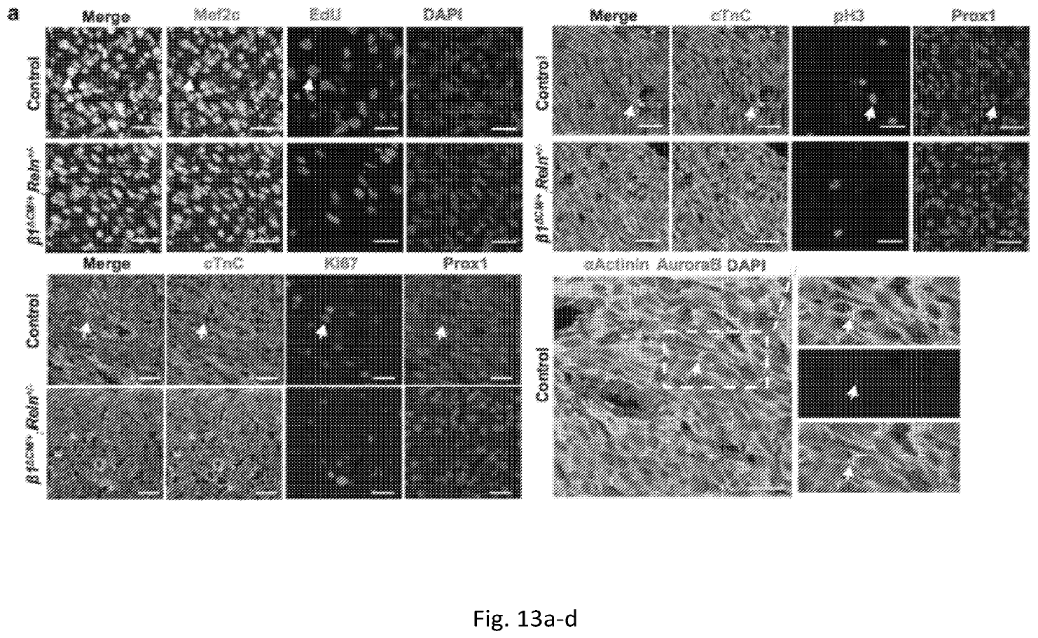

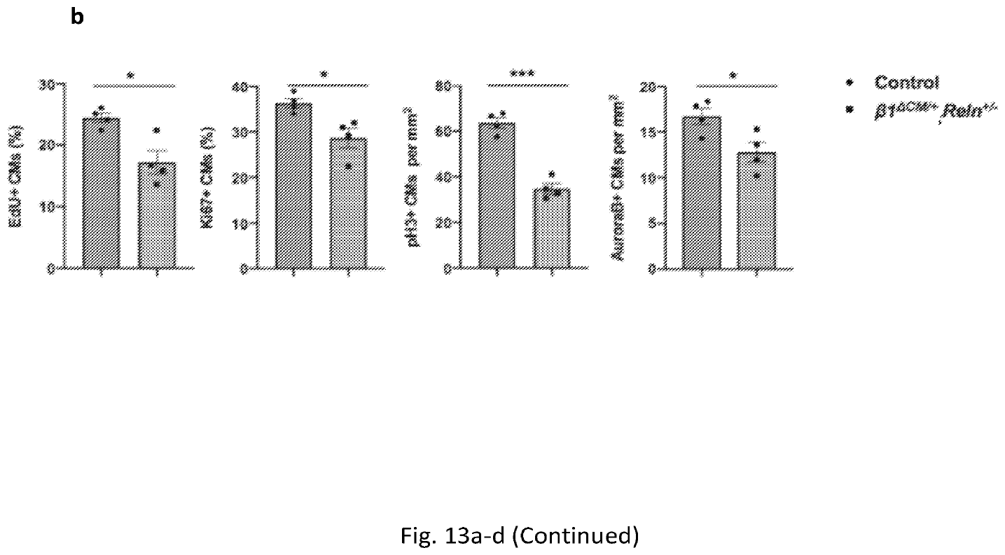

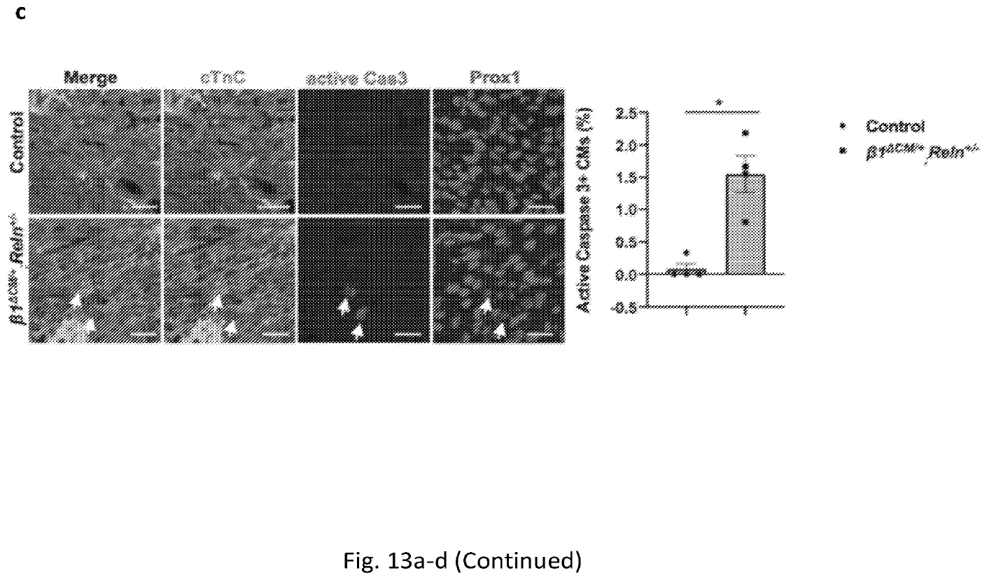

Previous studies about the role of Reelin during neuronal development, neuronal migration and in tumor cells identified VLDLR¹⁵, ApoER2^{20,21} and integrinβ1^{22,23} as Reelin receptors. Upon its binding to those receptors, Reelin stimulates intracellular signaling transduction through the phosphorylation of the intracellular protein Disabled-1 (Dab1) and the activation of PI3K/AKT/GSK3β²⁴ and mToR²⁵ signaling cascades. Integrinβ1 has been shown to play important roles in heart development, as its deletion in embryonic CMs results in smaller hearts with reduced CM proliferation²⁶. Therefore, the inventors investigated whether LEC-derived Reelin regulates CM proliferation and survival by regulating Integrinβ1 signaling. Western analysis confirmed that LEC conditioned media treated CMs increased the activity of Integrinol and Reelin downstream signals such as FAK, Dab1, AKT and ERK; in contrast, LEC conditioned media from Reln deficient LECs failed to induce Integrinβ1 signaling activity (FIG. 12b). More importantly, blocking Integrinβ1 signaling in CMs by adding Integrinol blocking antibodies to the LEC conditioned media partially abolished the pro-survival effects of the intact LEC conditioned media (FIG. 12b). Furthermore, LEC conditioned media from Reln deficient LECs or media containing Integrinβ1 blocking antibodies also failed to promote CM proliferation or to protect against CM apoptosis (FIG. 12c, d). These data further support the proposal that LEC secreted Reelin regulates CM proliferation and survival mainly by activating the Integrinβ1 signaling pathway. Furthermore, the inventors also observed an increase in Reelin/Integrinol signaling activity in mouse primary CMs upon Reelin stimulation (addition of supernatant from Reln transfected 293T cells), and this signaling was greatly inhibited by adding Integrinβ1 blocking antibodies (FIG. 12e). Moreover, E17.5 Integrinβ1^{f1/+}, MhcCre; Reln^{+/-} (β1^{ΔCM/+}; Reln^{+/-}) double heterozygous embryos generated by crossing Integrinβ1^{f1/+}, MhcCre (β1^{ΔCM/}) with Reln^{+/-} mice, also developed smaller hearts without significant size differences in livers or kidneys (FIG. 12f). Reln^{+/-} and β1^{ΔCM/+} littermates exhibited no differences in heart size compared to WT (FIG. 12f), and embryo size and cardiac lymphatics appear normal in all 3 genotypes resulting from those crosses (FIG. 12g). Consistently, CM proliferation was also significantly reduced and apoptosis increased in E17.5 β1^{ΔCM/+}; Reln^{+/-} embryos (FIG. 13a, b). No changes in proliferation and apoptosis were detected in other cardiac cell types or in kidney and liver (FIG. 13c). Together, these data support the proposal that LECs-secreted Reelin regulates CM proliferation and survival through the Integrinβ1 signaling pathway.

Neonatal Heart Repair Requires Reelin

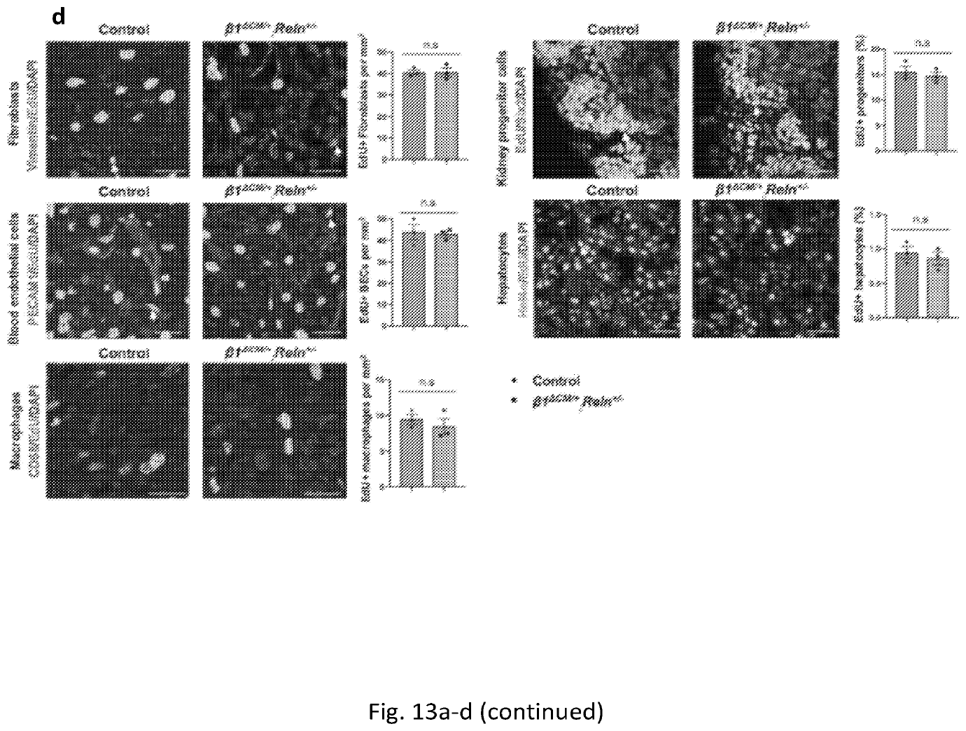

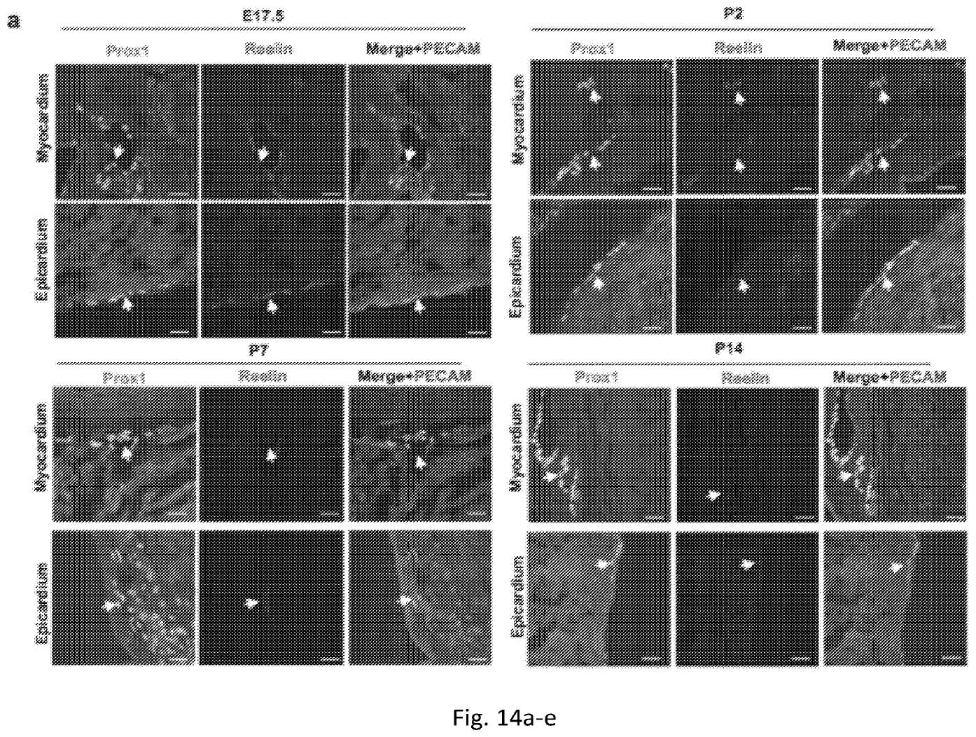

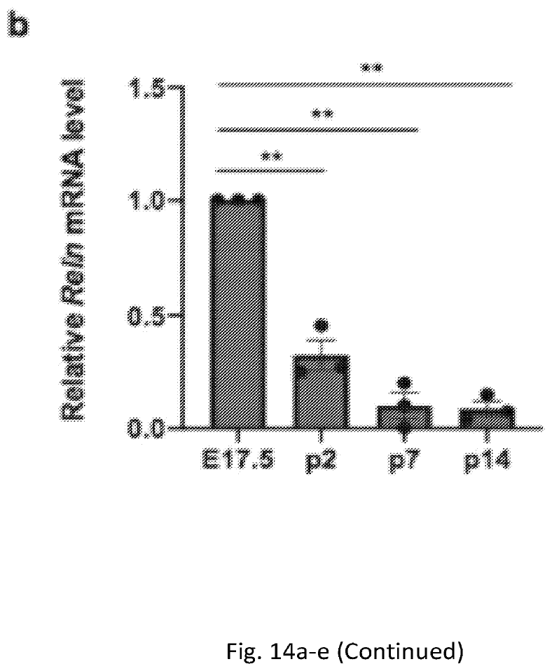

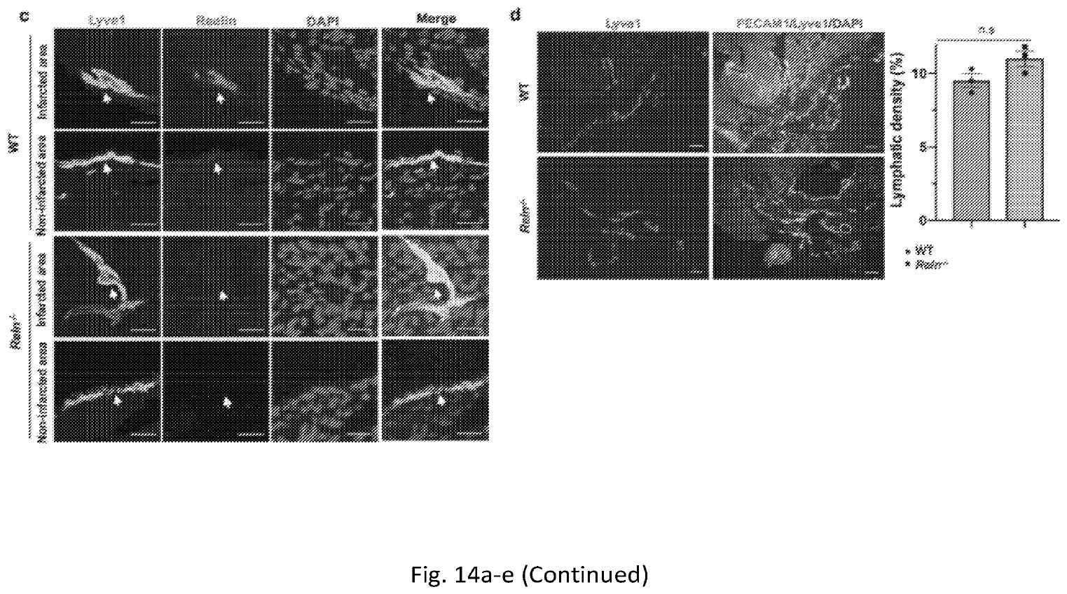

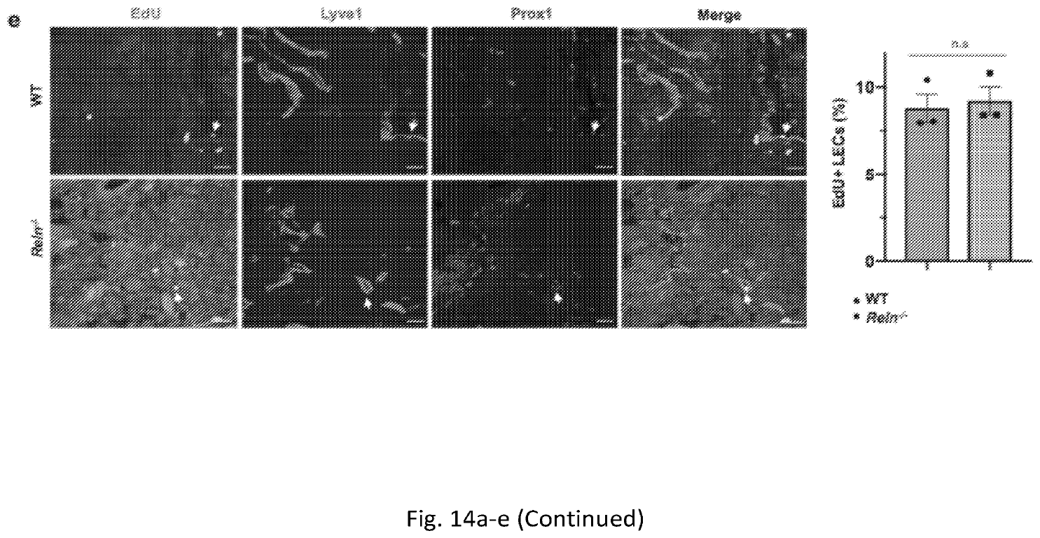

At E17.5, Reelin is highly expressed in cardiac lymphatics nearby the epicardium, as well as in the base of the myocardium; however, its expression levels get steadily reduced from P2 to P14, such that at P14 it is barely detected (FIG. 14a). This reduction in the levels of Reelin is accompanied by a similar change in the levels of Reln mRNA (FIG. 14b), suggesting that Reln expression levels are temporally regulated in cardiac lymphatics.

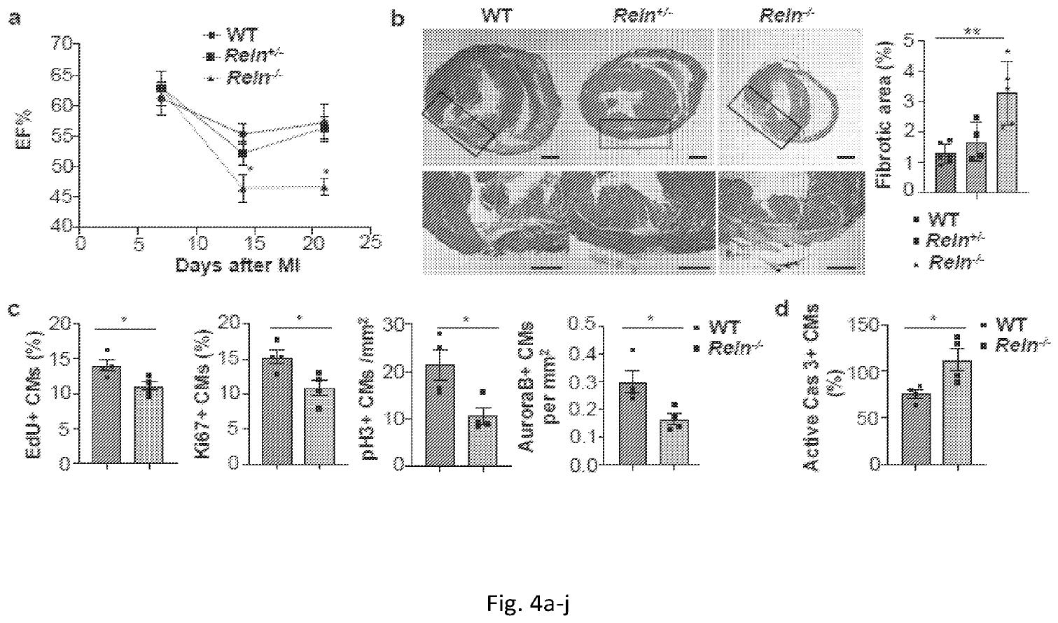

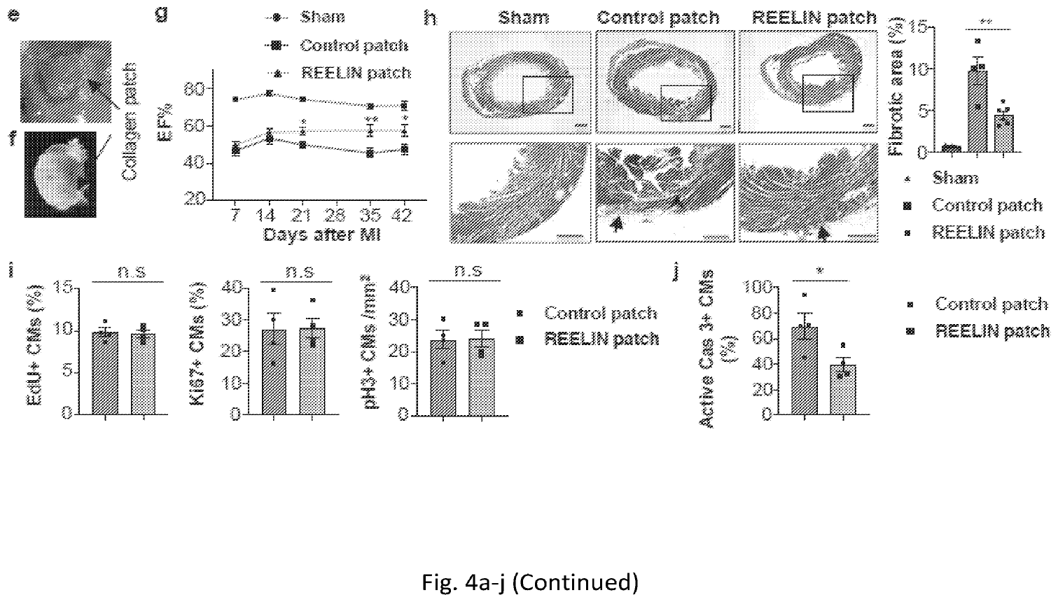

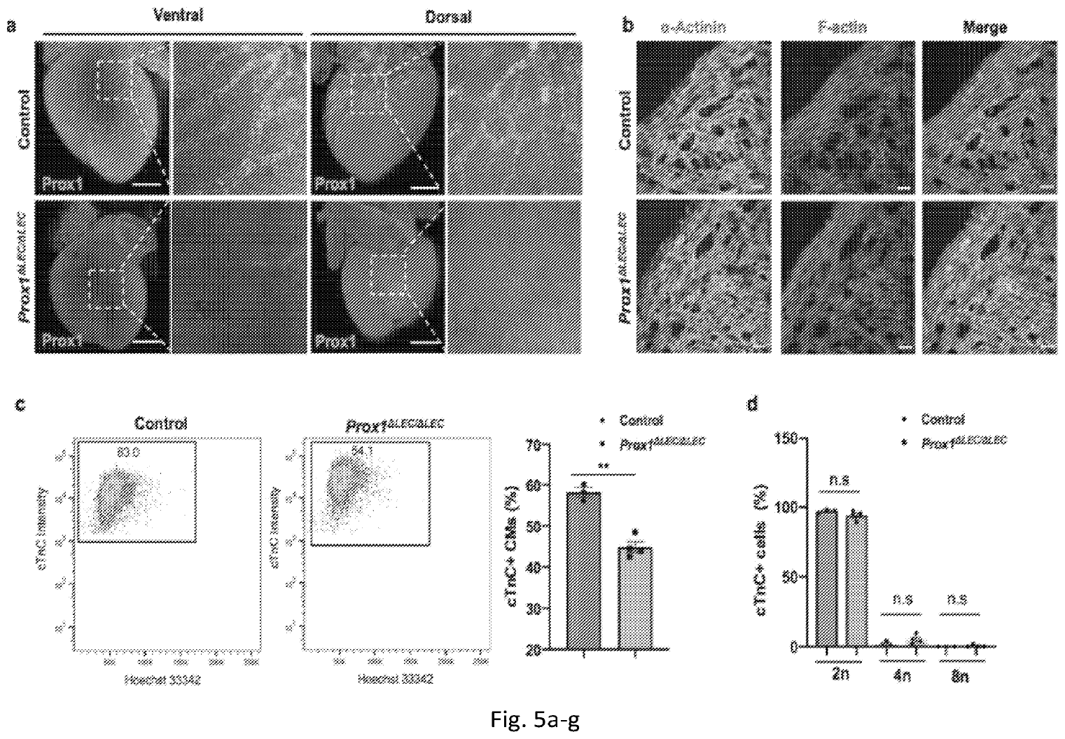

Since this reduction in Reelin expression coincides with the loss of cardiac regenerative potential in mice²⁷, the inventors first examined Reln role in WT mouse neonatal cardiac regeneration. The inventors performed neonatal MI at P2 and the analysis of P7 pups showed Lyve1-expressing lymphatics in both, the infarcted and the nearby non-infarcted cardiac tissue. Reelin expression was re-activated in the infarcted hearts, with higher levels in the infarcted area and low in the non-infarcted tissue (FIG. 14c). A similar analysis in P7 Reelin null pups showed that similar to WT controls, Lyve1-expressing lymphatics were present in the infarcted and non-infarcted tissues, although in both cases Reelin expression was not detected (FIG. 14c). Compared to WT controls, although cardiac function was not affected in Reln^{-/-} hearts at P7, it was reduced at P14 and P21, as determined by echocardiography (FIG. 4a). In line with this reduced cardiac function, Masson's trichrome staining showed increased fibrosis in P21 Reln^{-/-} hearts (FIG. 4b). Immunostaining revealed that neither lymphatic density nor LEC proliferation is affected in P21 Reln^{-/-} hearts after injury (FIG. 14d, e). Similar to the results observed in E17.5 Reln null embryos, CM proliferation was reduced and CM apoptosis was increased in the infarcted area of P7 Reln^{-/-} hearts (FIG. 4c, d, 15a-e). Importantly, no alterations in cardiac function or increased fibrosis were seen in Reln^{-/-} mice (FIG. 4a-b). Immunostaining revealed that neither lymphatic density nor LEC proliferation is affected in Reln^{-/-} hearts after injury (FIG. 14d, e). These results demonstrate that Reelin re-expression in cardiac-associated lymphatics of the injured neonatal heart improves cardiac regeneration and function after MI.

Reelin Improves Adult MI Recovery

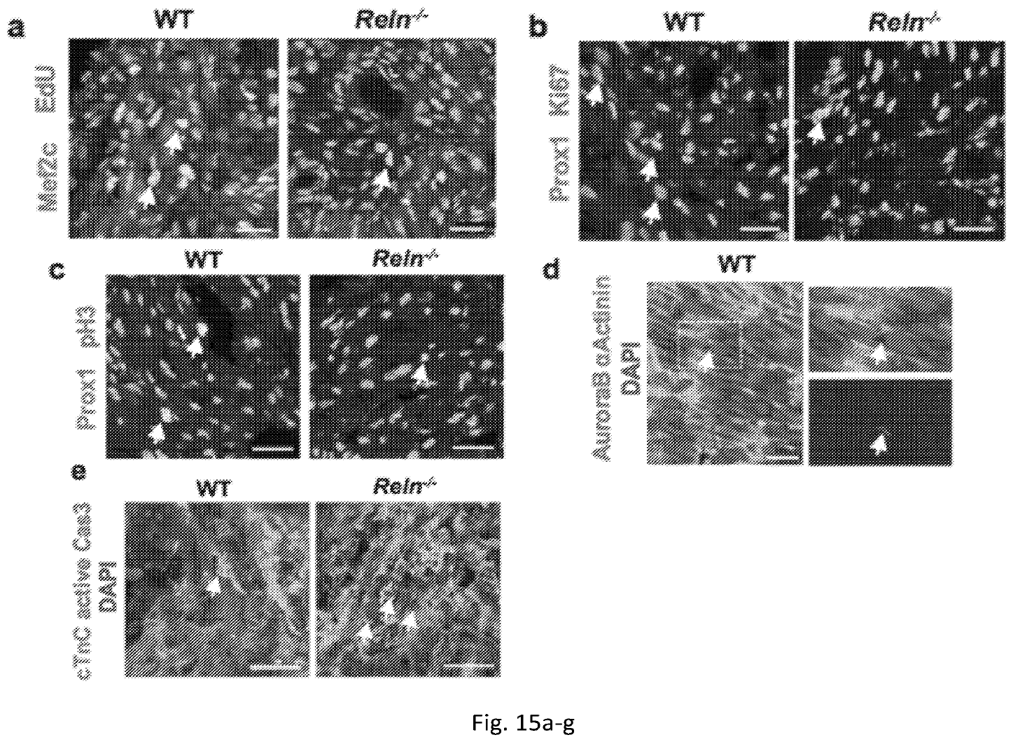

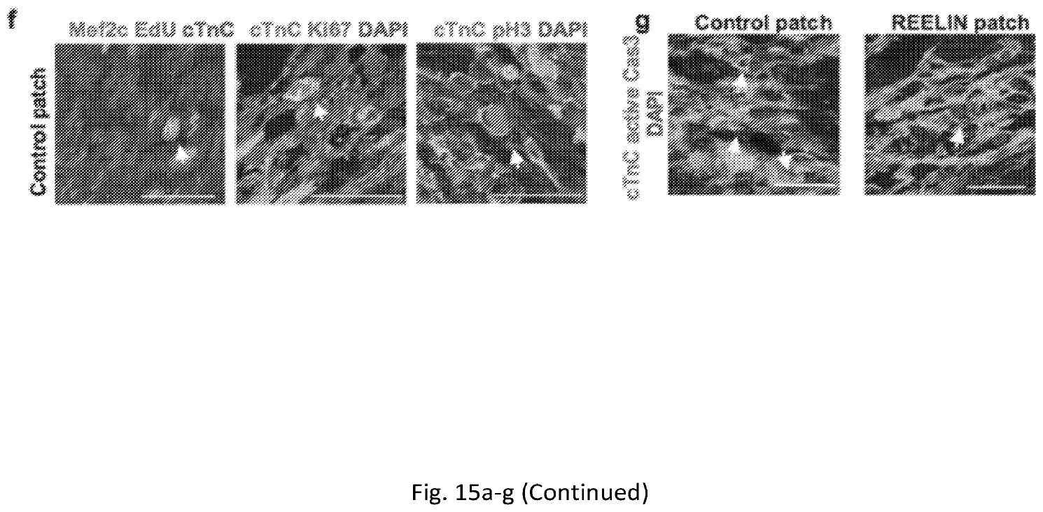

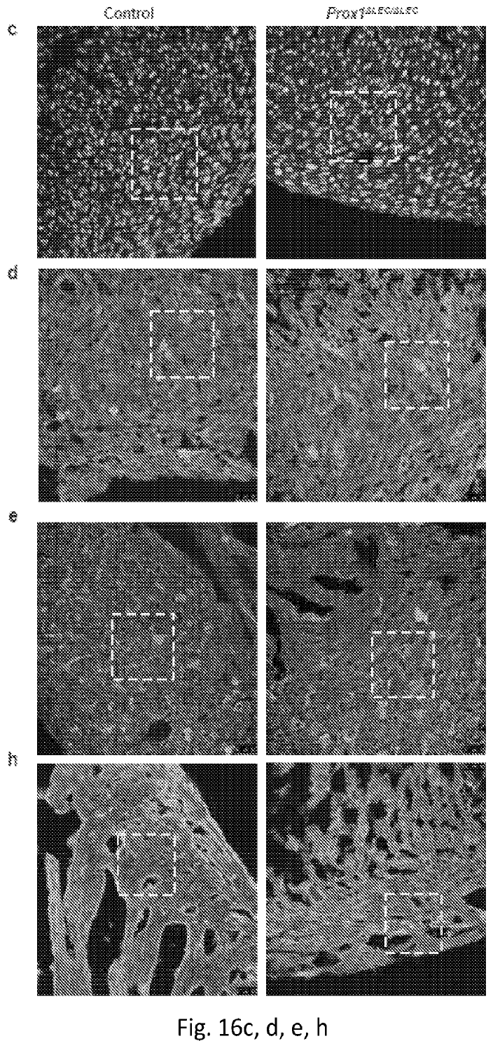

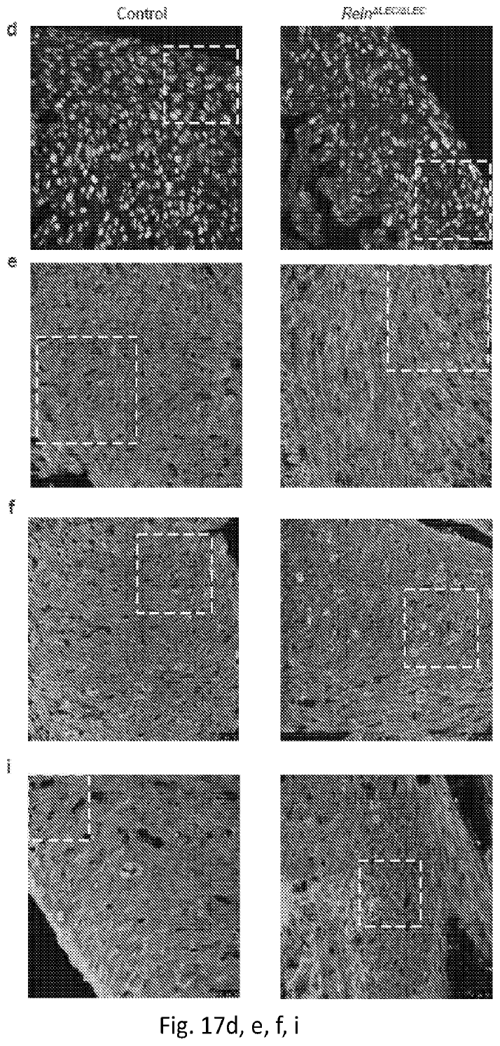

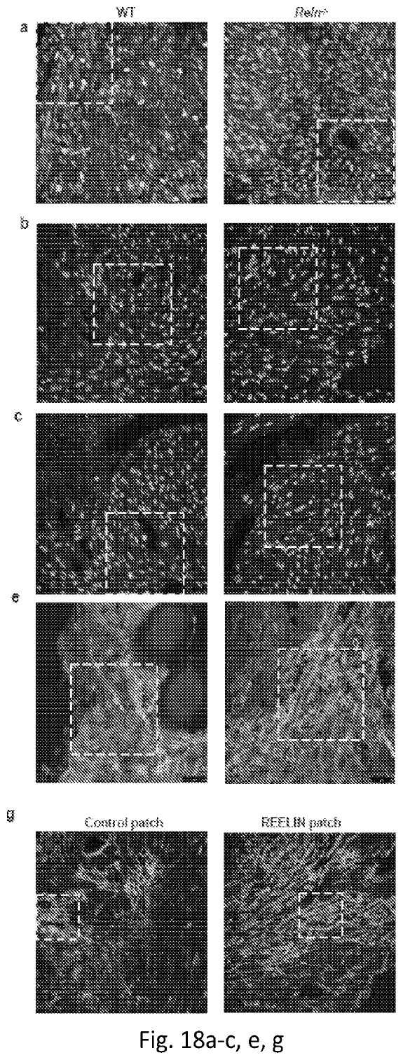

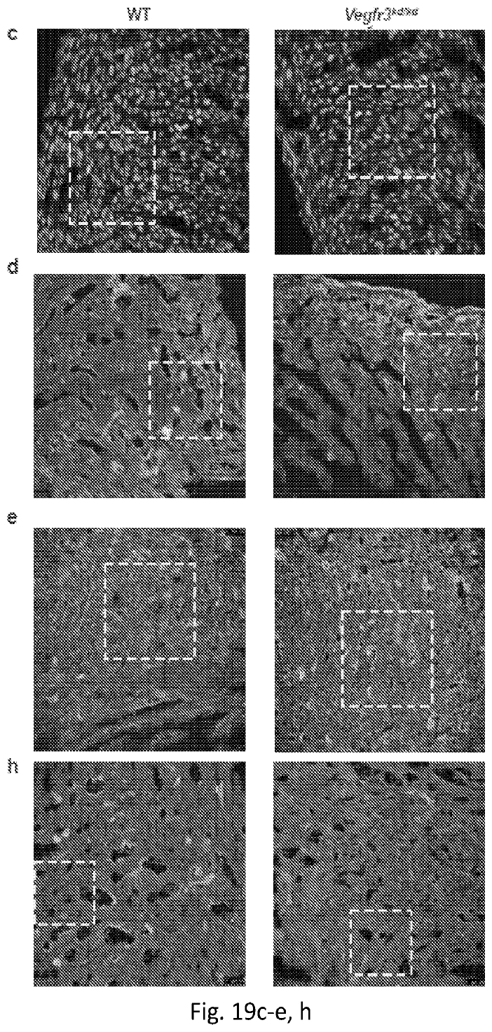

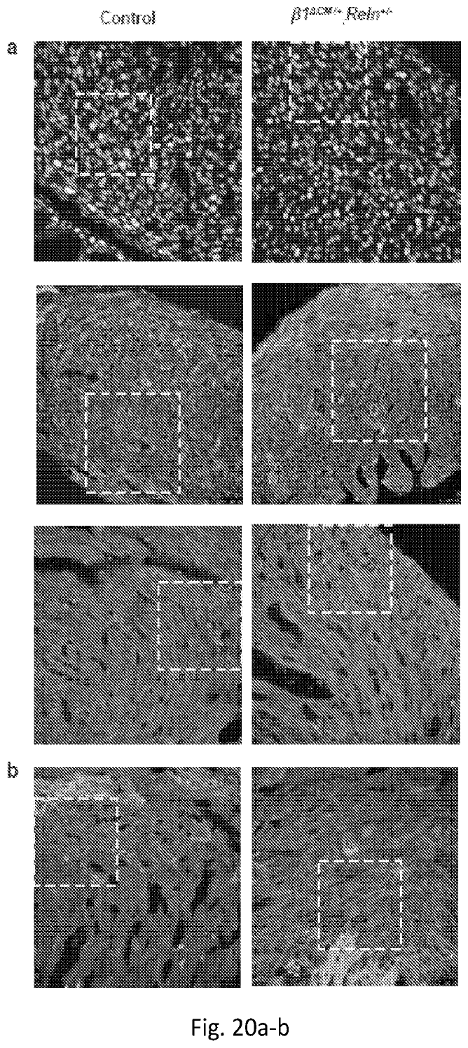

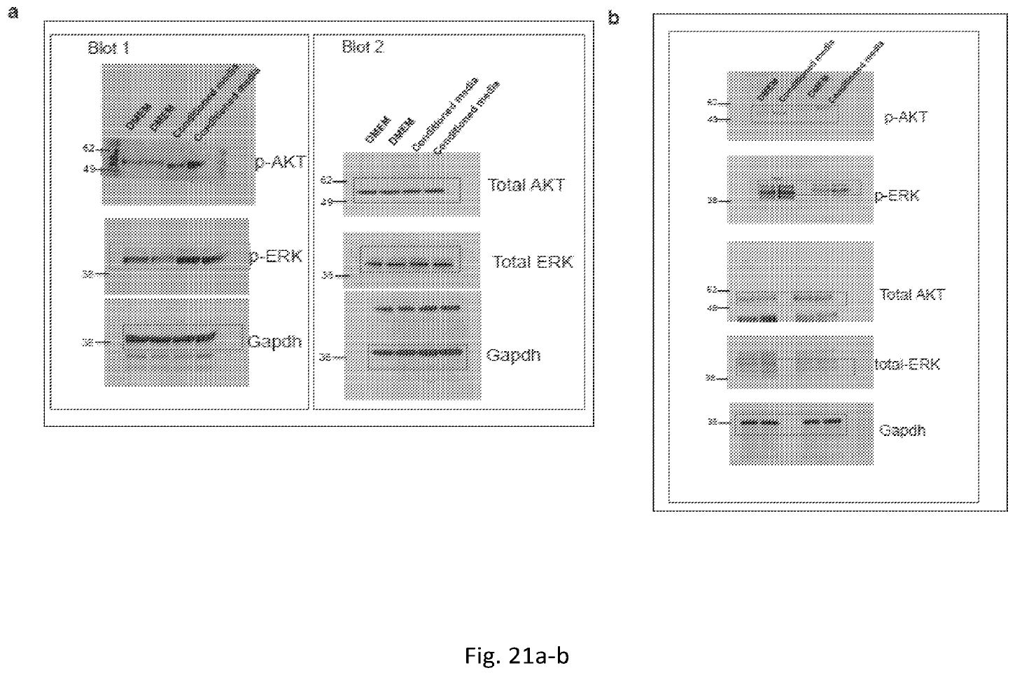

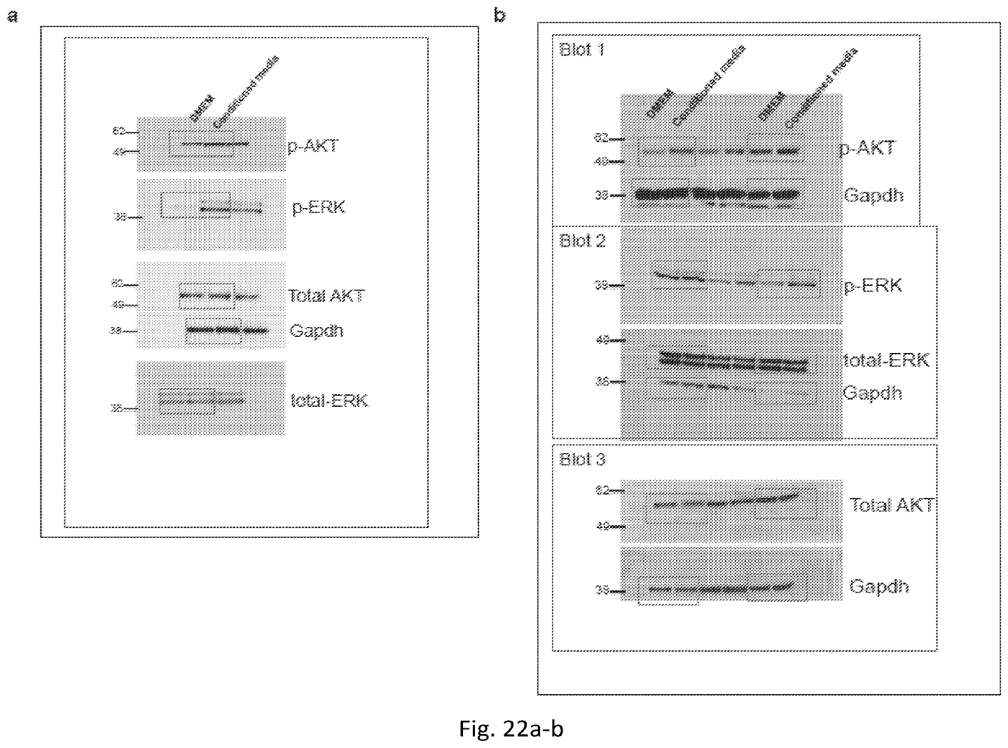

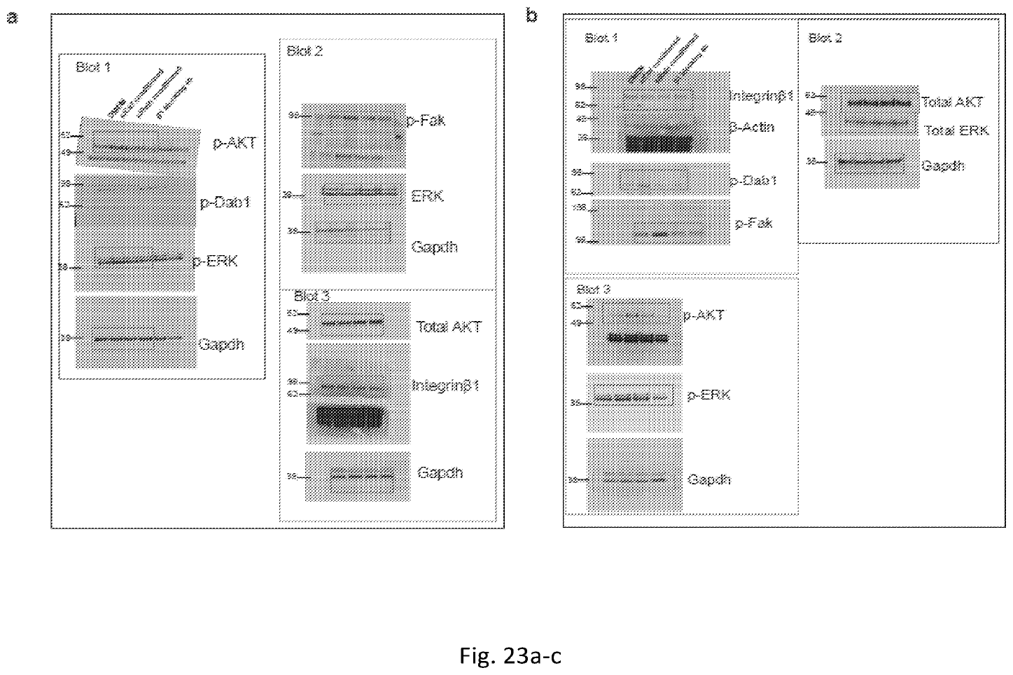

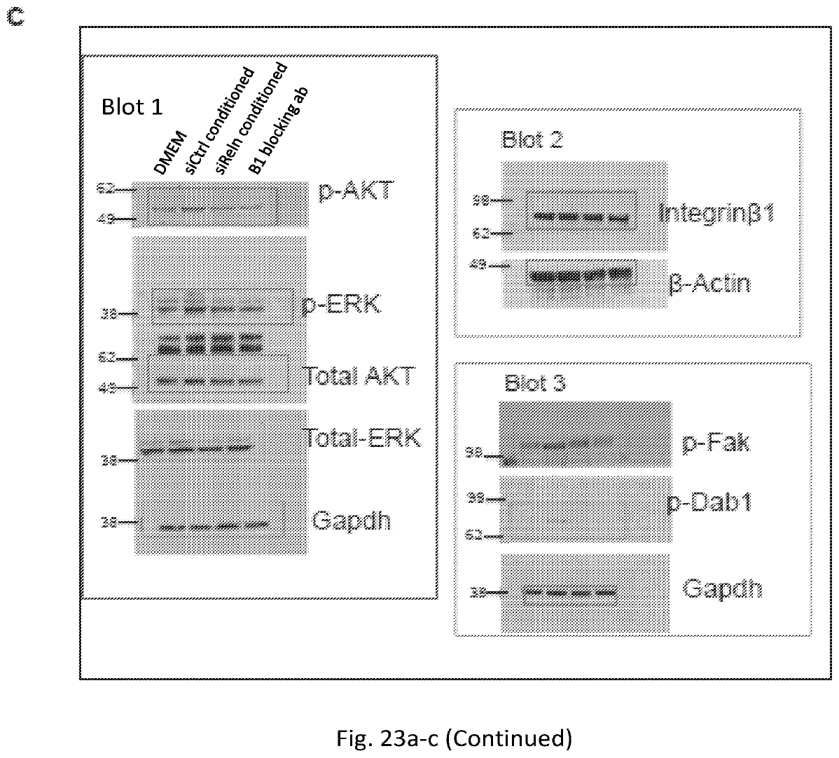

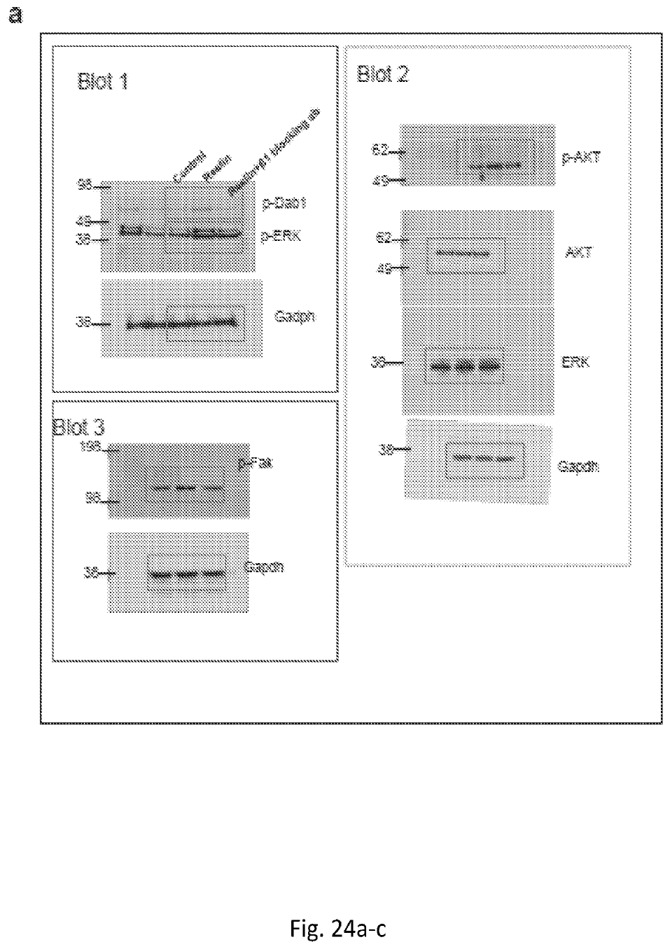

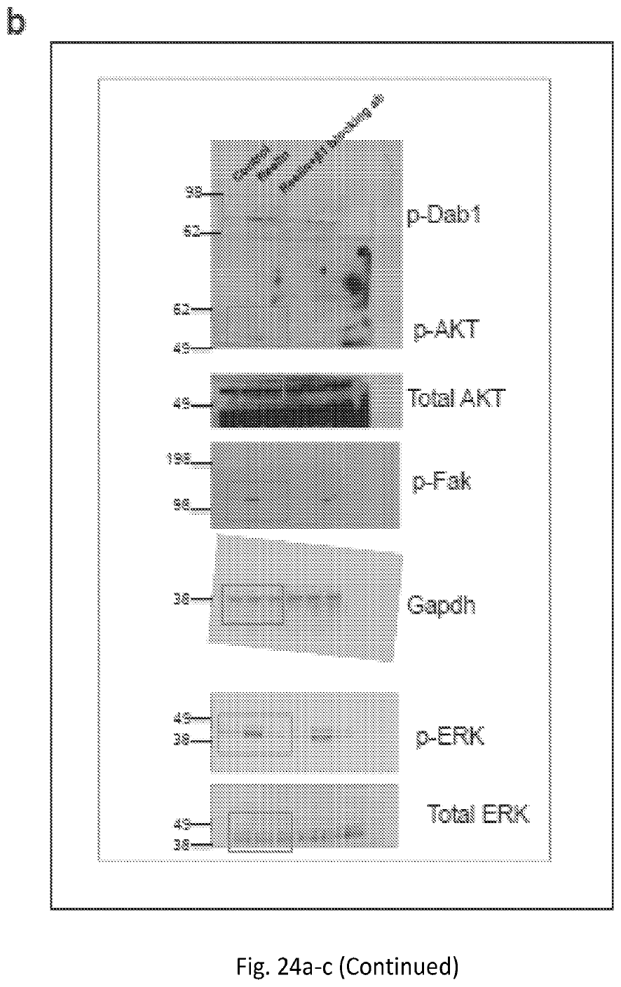

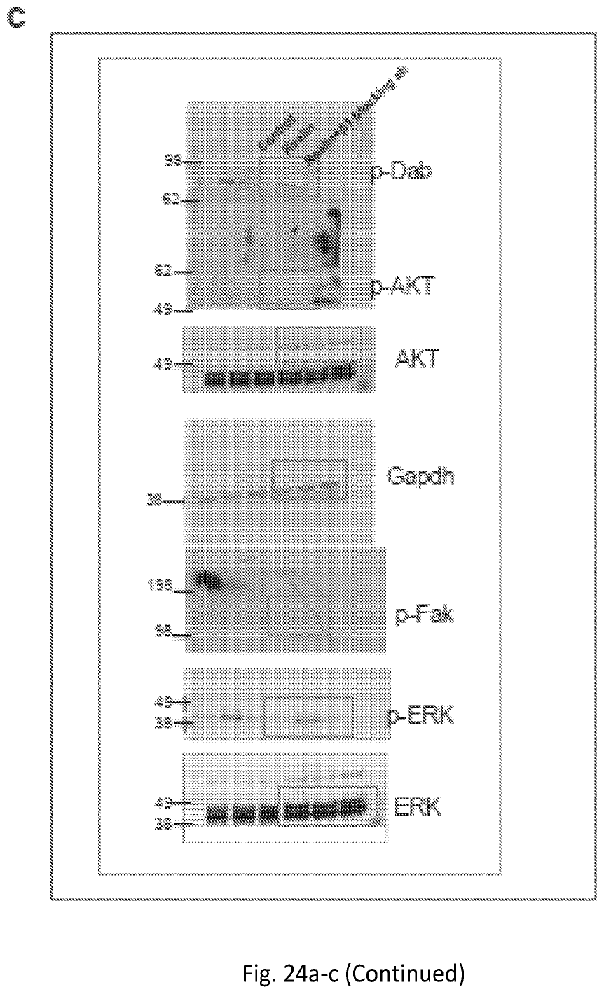

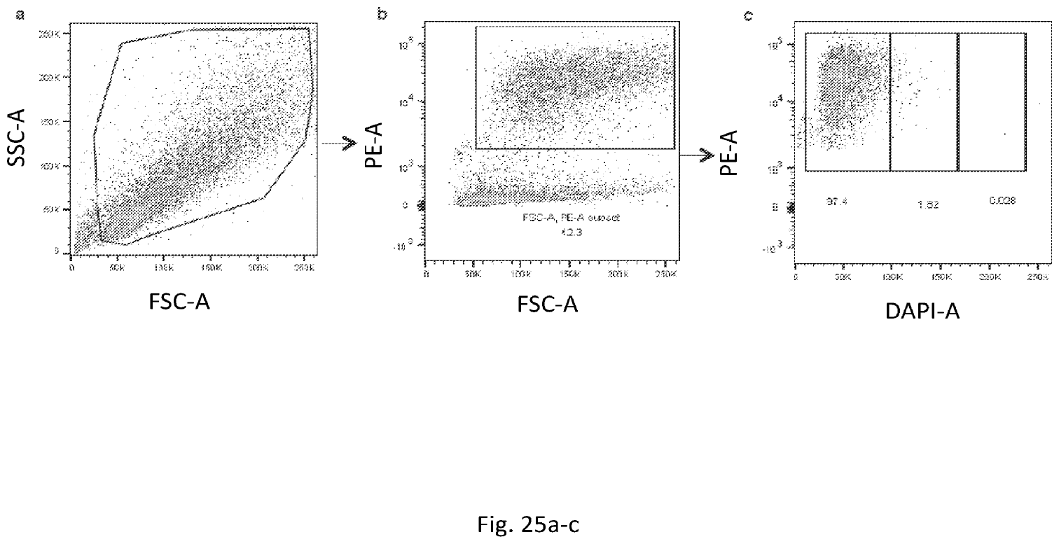

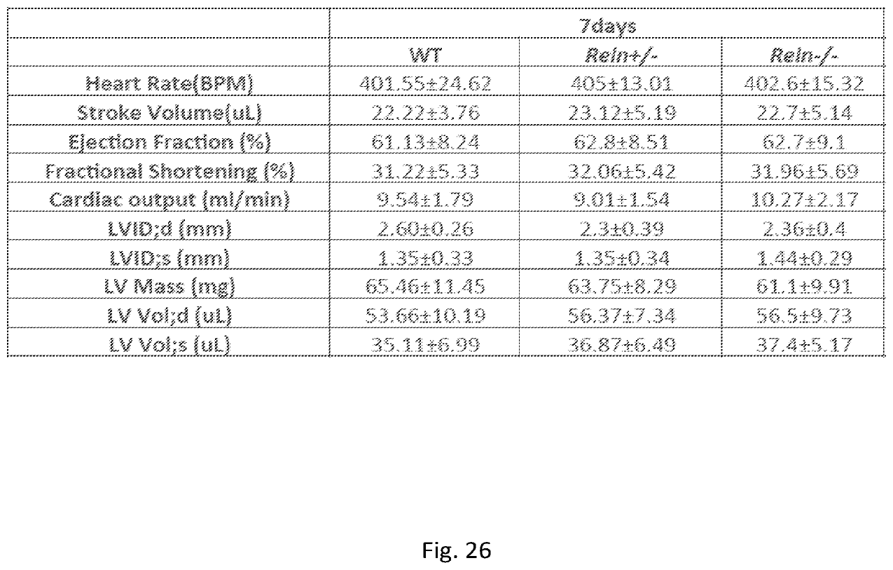

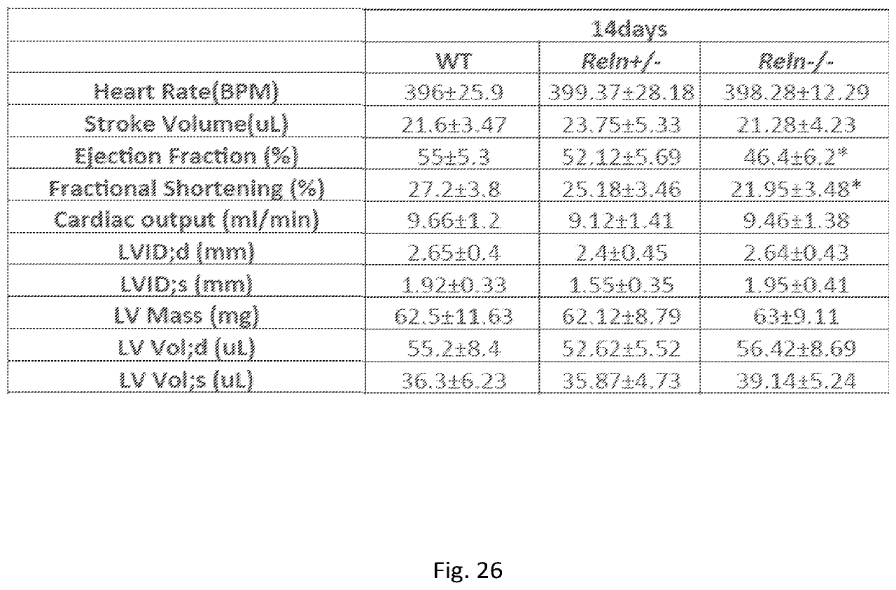

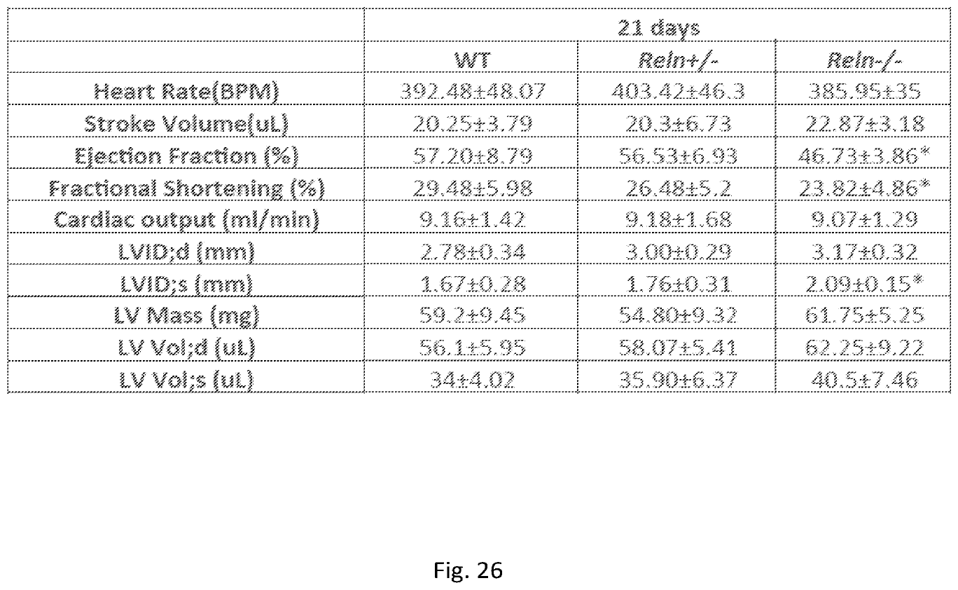

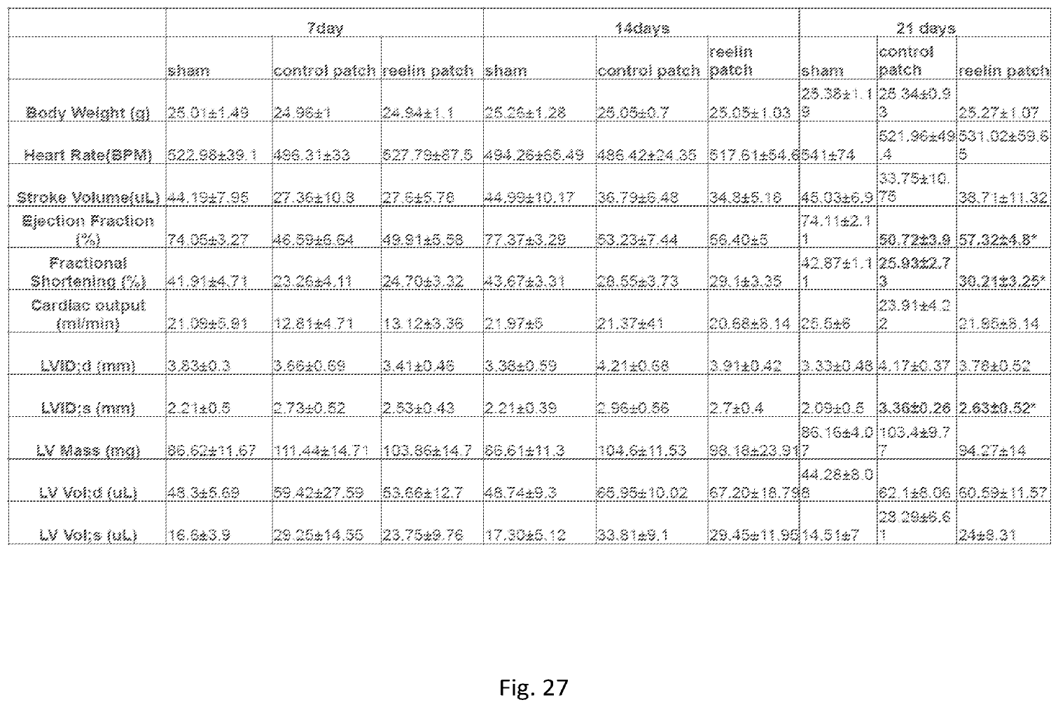

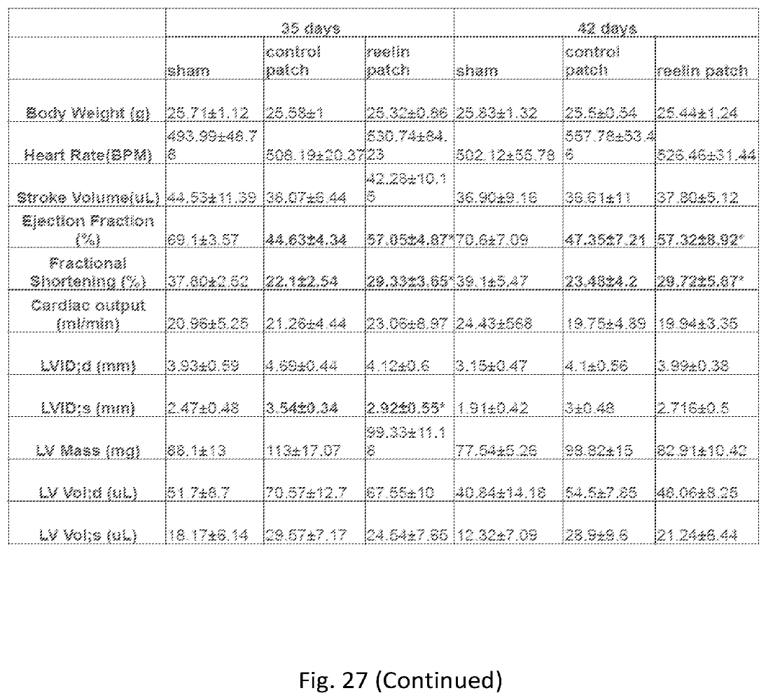

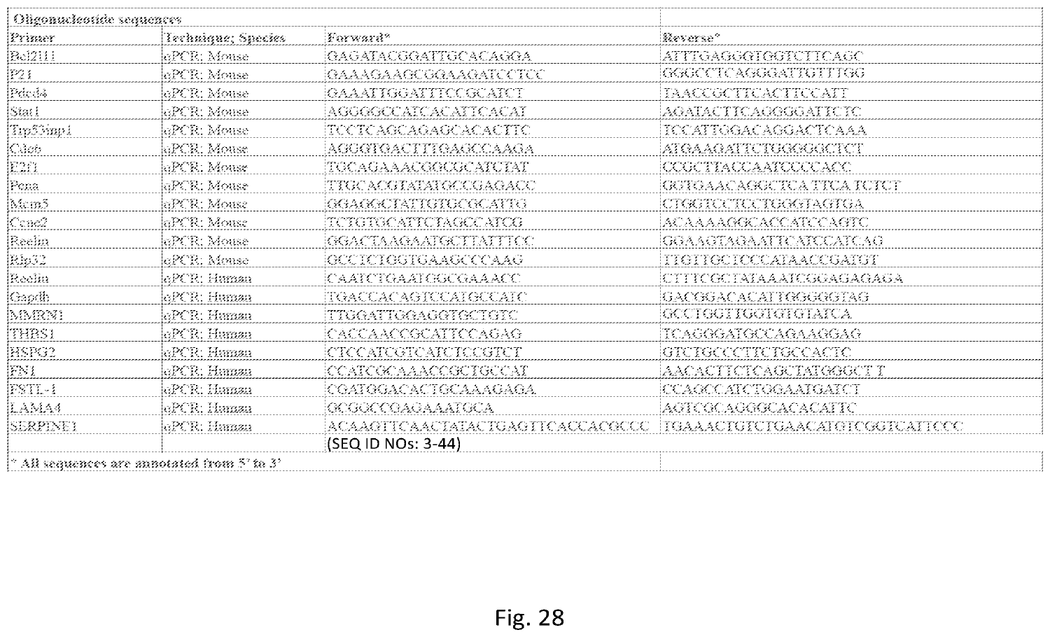

The inventors next assessed whether delivery of Reelin directly into the heart could improve cardiac repair in adult WT mice after MI. The inventors took advantage of well-established bioengineered collagen patches^{28,29} as a scaffold to deliver recombinant REELIN protein into the heart. REELIN-containing patches and control patches were surgically sutured onto approximately 2-month old injured hearts immediately following acute MI (FIG. 4e, f). Cardiac function was evaluated weekly (1-6 weeks after MI) and as shown in FIG. 4g, 21 days after MI ejection fraction (EF) was significantly improved in mice with REELIN patches. Consistent with this improved heart function, 42 days after MI the size of the fibrotic scar in the infarcted area was remarkably reduced in REELIN-patched mice (FIG. 4h). To evaluate whether this improved cardiac function and reduced fibrotic tissue was consequence of increased CM proliferation and/or reduced CM cell death, the inventors performed immunostaining seven days after MI, a stage when increased CM proliferation is normally detected after injury. As seen in FIG. 4i and FIG. 15f, no differences in CM proliferation were observed in the infarcted area between mice with control or REELIN patches as indicated by EdU labeling and Ki67 or pH3 immunostaining. Importantly, CM apoptosis was greatly reduced in the infarcted area of REELIN patched mice (FIG. 4j, FIG. 15g). These data indicate that following adult cardiac injury, REELIN protects CMs from apoptosis, which correlates with a reduced scar and improved heart function.

### DISCUSSION

Using mouse embryos devoid of LECs or Reelin-producing LECs, the inventors demonstrate that their hearts are smaller as a consequence of increased CM apoptosis and reduced CM proliferation. The inventors showed that the percentage of CMs is significantly reduced in E17.5 Prox1^{ΔLEC/ΔLEC} and Reln^{ΔLEC/ΔLEC} hearts, suggesting that communication between LECs and CMs is required for CM survival during cardiac development. The inventors also found that LEC-conditioned medium increases CM survival and prevents CM apoptosis consequence of hypoxia; a result suggesting potent LEC lymphoangiocrine cardioprotective effects. The inventors identified Reelin as a factor performing such functional role likely via the Integrinβ1 signaling pathway, both in vivo and in vitro. Finally, the inventors provide some additional insight about the proposed beneficial roles of lymphatics on cardiac repair by showing that at least partially, is mediated by Reelin activity. The inventors demonstrate Reelin relevance in the endogenous cardiac regenerative ability by showing that following MI at P2, Reelin expression in LECs is particularly reactivated in the MI area of wild-type mice, and that Reln^{-/-} mice do not fully regenerate. The inventors found that Reelin is required for CM proliferative activity at P7, although proliferation was not completely abolished in Reln^{-/-} pups, indicating that other factors contribute to CM proliferation. Cardiomyocytes apoptosis was also increased in Reln^{-/-} mice during an extended period after MI (up to P21), suggesting that in addition to the reduced proliferation, loss of CM protection underlies the inability of Reln^{-/-} postnatal hearts to fully regenerate.

The inventors also demonstrate that exogenously applied Reelin is useful for cardiac repair in the adult heart after MI. Whereas during cardiac growth and in neonatal cardiac regeneration Reelin promotes both, CM proliferation and survival, in the adult heart Reelin beneficial activity on cardiac function seems to be mostly consequence of reduced CM cell death and a smaller scarred myocardial area, both features indicative of a cardioprotective effect.

Although these results argue that Reelin regulation of Integrin mediated signaling is specifically critical for CM proliferation and survival, it is likely that alternative signals or receptors mask similar effects on other cardiac cell types such as fibroblasts and BECs. Furthermore, it is also likely that Reelin and/or other lymphoangiocrine signals play similar homeostatic roles in other organs.

In summary, this study highlights the importance of LECs and REELIN during heart growth and repair, and provides some ideas about possible paths to improve cardiac regeneration and cardio-protection in mammals. These results suggest that the use of REELIN could be a valuable therapeutic approach to improve cardiac function in humans.

Reelin Null Mouse

The experiments above determined that a) Reelin is required for normal cardiac growth during development b) is required for neonatal heart regeneration as Reelin null hearts fail to properly regenerate c) Reelin improves cardiac function after MI as addition of collagen patches containing Reelin after MI in adult mice improve cardiac repair.

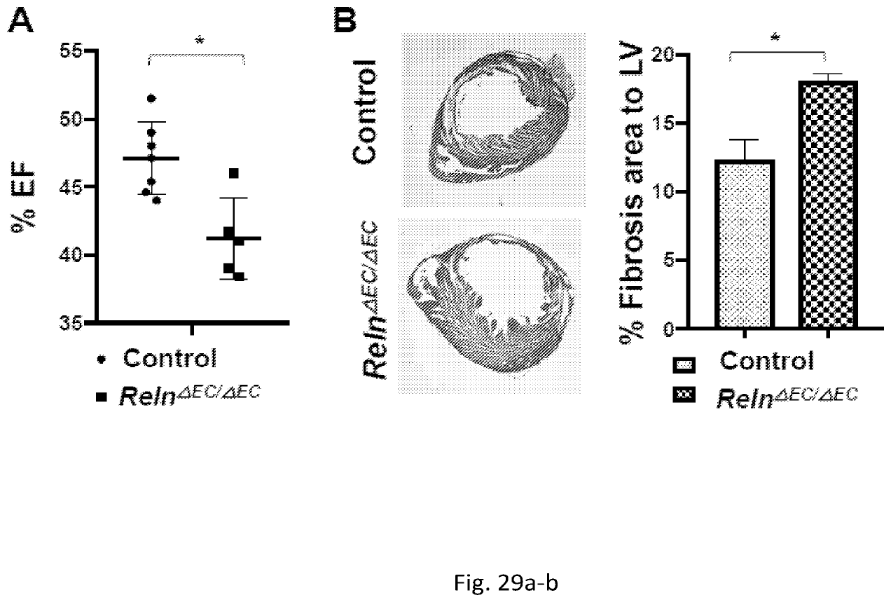

To further validate and expand those results, particularly those about the role of Reelin in cardiac repair in the adult heart after MI, conditional null Reelin adult mice were generated, where Reelin was specifically removed from lymphatic endothelial cells (LECs) by crossing Reln floxed mice with VE-CadCreERT2 mice. Considering that in endothelial cells, Reln is mainly expressed in LECs, that cross generated LEC-specific Reln conditional null adult mice following tamoxifen injections in 6-8 weeks old mice [VE-CadCreERT2,Reln^{f/f}(Reln^{ΔEC/ΔEC})]. Acute myocardial infarction (MI) was performed then two weeks after tamoxifen injections in Reln^{ΔEC/ΔEC} and littermate controls. Four weeks later, echocardiography revealed significantly impaired cardiac function (EF %) in Reln^{ΔEC/ΔEC} mice compared to littermate controls. Masson's trichrome staining also shows increased cardiac fibrotic area in Reln^{ΔEC/ΔEC} mice 4 weeks post-MI. Quantification of the percentage of fibrotic area is shown in the right panel.*p<0.05 by unpaired student t test. FIG. 29.

Methods

Mouse Models

LEC-specific Prox1 deficient mice were generated by crossing Prox1^{f/f} mice7 with Cad5(PAC)-CreER^{T2} mice¹⁰. These mice are maintained in a mixed C57B6 and NMRI background. LEC-specific Reln deficient mice were generated by crossing Reln^{f/f} mice¹⁸ with Prox1CreER^{T2} mice¹⁹. These mice are in a mixed 129, FVB and C57B6 background. Reln^{+/-} mice were kindly provided by Dr. Bianka Brunne and are originally from the Jackson laboratory and are maintained in a mixed Balb/c and C57B6 background. For induction of Cre mediated recombination in Prox1^{ΔLEC/ΔLEC} and Reln^{ΔLEC/ΔLEC} embryos, two consecutive intraperitoneal tamoxifen (TAM) injections of 5 mg/40 g were administered to pregnant dams. IntegrinΔ1^{f/f} mice and MhcCre mice were obtained from the Jackson laboratory and are in a mixed C57B6 and NMRI background. These strains were bred to generate MhcCre; Integrin β1^{f/f} mice that were crossed with Reln^{+/-} mice to obtain MhcCre; Integrinβ1^{f/f}; Reln^{+/-} (β1^{ΔCM/+}; Reln^{+/-}) embryos. Heterozygous mice carrying the kinase-dead Flt4^{Chy} allele (Vegfr^{3kd}) (MRC Harwell) were described previously³⁰ and are maintained in the NMRI background. Twelve-weeks-6 month old mice of both sexes were used for breeding and experiments. Mice were not randomized into experimental groups, but were age and sex-matched and littermates were used whenever possible. All animal husbandry was performed in accordance with protocols approved by Northwestern University and UT Southwestern Medical Center Institutional Animal Care and Use Committee, as well as Animal Experimentation Review Board of the Semmelweis University. Animal facilities are equipped with a 14:10 or 12:12 light cycle. Temperatures are maintained between 18-23° C. with 40-60% humidity.

Mouse Embryonic CM Isolation

CMs were isolated from E15.5-17.5 mouse embryos using the Pierce Primary Cardiomyocyte Isolation Kit (Thermo Fisher). Briefly, ventricles were isolated from embryonic hearts and minced and washed with cold HBSS and further digested according to the manufacture instructions. To examine the relative CM cell size, dissociated cells were cultured in DMEM containing 10% FBS o/n and then cells were fixed in 4% PFA for immunostaining. For any other experiments, primary cells were cultured in DMEM containing 10% FBS and cardiomyocyte growth supplements for 3-4 days before experiments.

Human iPSC Derived-CMs (hiPSC-CMs)

Cardiac differentiation was performed using the CDM3 (chemically defined medium, three components) system as described with slight modifications^{31,32} hiPSCs are split at 1:15 ratios and grown in B8 medium for 4 days reaching ˜80% confluence. On day 0, B8 medium is changed to CDM3³¹, consist ng of RPMI 1640 (Corning, 10-040-CM), 500 μg/ml fatty acid-free bovine serum albumin(GenDEPOT), and 200 μg/ml 1-ascorbic acid 2-phosphate (Wako), supplemented with 6 μM of CHIR99021 (LC Labs, C-6556). After 24 hours (day 1), medium is changed to CDM3. On day 2, medium is changed to CDM3 supplemented with 2 μM of Wnt-C59 (Biobyt, orb181132). Medium is then changed every other day for CDM3 starting on day 4. Contracting cells are noted from day 7. On day 16 of differentiation, CMs are dissociated using DPBS for 20 min at 37° C. followed by 1:200 Liberase TH (Roche) diluted in DPBS for 20 min at 37° C., centrifuged at 300 g for 5 min, and filtered through a 100 μm cell strainer (Flacon). The purity of the differentiated cells was determined by expression of CM cell marker TNNT2 using flow cytometry. Only cell lines that show over 85% are TNNT2+ were used for experiments.

LEC-Conditioned Medium

Human dermal LECs were purchased from Lonza and cultured with endothelial basal medium (EBM) complemented with supplement mix (Lonza). Passages 4 or 5 were cultured in 10 cm dishes until confluent, washed with cold PBS three times and then 8 ml of serum free DMEM (without phenol red) with penicillin/streptomycin was added. Cells were then cultured o/n before collecting the conditioned media that was filtered through a 0.22 μm pore membrane (Millipore). Control conditioned media (DMEM) was prepared in the same way but without LECs.

siRNA Knockdown

Human LECs were transfected as described previously³³. Briefly, P4 human LECs were transfected with scrambled or Reln siRNA (Santa Cruz) with Lipofectamine 2000 (Invitrogen), according to the manufacture's instruction. After 48 h, cells were washed and replaced with DMEM and further cultured o/n to collect the conditioned media. LECs were collected and qPCR was performed to check transfection efficiency.

LEC-Conditioned Media Treatment

To examine the effects of the LECs conditioned media, mouse primary CM or human iPSC-CM were cultured in 12 well plates (about 80% confluence), and cells were treated either with DMEM, conditioned media, conditioned media from scrambled siRNA treated LECs (siCtrl-conditioned), conditioned media from siReln treated LECs (siReln− conditioned) or conditioned media with Integrin 31 blocking antibodies (10 μg/ml, BD Biosciences) o/n. Cells were either fixed in 4% PFA for immunofluorescent staining, or lysed in RIPA buffer for Western blot analysis.

Reelin Conditioned Media and Treatment

HEK-293T cells (ATCC) were cultured in DMEM with 10% fetal bovine serum and transfected with the Reelin cDNA construct pCrl, kindly provided by Dr. Gabriella D'Arcangelo using Lipofectamine 2000 (Invitrogen). Control cells were mock transfected in the same way without adding the vector. Twenty-four hours after transfection, the medium was changed to serum free DMEM and Reelin conditioned medium and mock conditioned media (control) were collected two days after the medium change. The conditioned medium was filtered through a 0.22 μm pore membrane. To examine the effects of the Reelin conditioned media, mouse primary CM were starved o/n with DMEM and stimulated for 30 min with Reelin conditioned media (supernatant from transfected cells) or control media (supernatant from mock-transfected cells). To examine the Reelin/Integrinp 1 pathway, primary CM were treated in the presence or absence of Integrin β1 blocking antibodies (10 μg/ml, BD Biosciences) for 3 h prior to Reelin conditioned media treatment.

Western Blot Analysis

To examine signaling changes in primary CMs or iPSC-CMs, cells were lysed in RIPA buffer and subject to Western blot analysis. The following primary antibodies were used: p-AKT (Rabbit, Cell Signaling, 4060, 1:500), p-ERK (Rabbit, Cell Signaling, 4370, 1:1,000), total AKT (Rabbit, Cell Signaling, 4691, 1:500), total ERK (Rabbit, Cell Signaling, 4695, 1:500), p-Dab1 (Rabbit, Cell Signaling, 3327S, 1:100), p-FAK (Rabbit, Cell Signaling, 3284 1:200), integrin 31 (Mouse, BD, 610467, 1:100), Gapdh (Rabbit, Santa Cruz, sc32233, 1:5,000). Blots were imaged using a ChemiDock imaging system (Bio-Rad) and bands were acquired using Quantity One 1-D software. Quantification of Western blot was analyzed using ImageJ 1.51. Included images are representative blots. All raw data used for the quantifications is included in the Figures.

Mass Spectrometry Analysis of LEC Conditioned-Media

50 ml of LEC-conditioned was collected from five 10-cm dishes of cultured LECs and filtered through a 22 μm pore membrane as mentioned above. LEC-conditioned media was further concentrated into 500 uls using the Protein-Concentrate Kit (Millipore) according to the manufacture's instruction. Protein concentration was then measured by the BCA protein assay (Thermo Fisher). Experiments were repeated three times and three biological samples were submitted to Northwestern Proteomics Core for untargeted quantitative proteomics analyses by Label-free Quantitative Proteomics: Briefly, samples were analyzed using an UltiMate™ 3000 RSLCnano system (ThemoFisher Scientific, CA) that is coupled with electrospray ionization (ESI) to a linear ion trap (LTQ) Orbitrap mass spectrometer (iLTQ-Orbitrap, ThermoFisher, CA). The resulting raw mass spectra from all three replicates were analyzed by the MaxQuant search engine (version 1.6.0.16) using UniprotKB human database with the allowance of up to 2 missed cleavages and precursor mass tolerance of 20 p.p.m. The secretome was acquired using software Scaffold 4 and annotated using Gene Ontology (GO), which assigns putative cellular compartmentalization, biological process and molecular functions.

ELISA

To validate the presence of Reelin in the LECs conditioned media, 3 different batches of commercial LECs were cultured and their conditioned media was collected as described. Sandwich enzyme-linked immunosorbent assay (ELISA) was performed to examine the relative levels of Reelin in the 3 different batches of LECs conditioned media. Briefly, conditioned media were pre-coated to Nunc MaxiSorp™ Flat-Bottom 96-well plates (Invitrogen) o/n and blocked with 5% milk in TBST. Plates were then incubated with Reelin primary antibody (R&D, AF3820, 1:100) and followed by incubation with HRP conjugated Donkey anti-goat antibody (Jackson ImmunoResearch, 705-035-003, 1:1000). Subsequently, plates were washed and the substrate solution (3,3,5,5-tetramethylbenzidine liquid substrate system for ELISA, Abcam) was added. The reaction was stopped by adding 2N H₂SO₄, and plates were measured at 450 nm using the Opsys Mr microplate reader (Dynex Technologies). Relative Reelin levels in different batches of conditioned media were quantified by OD intensity.

FACS Analyses and Sorting

For analysis of percentages of CMs in the heart, whole E17.5 ventricles were dissociated from control and Prox1^{ΔLEC/ΔLEC} hearts using the Pierce Primary Cardiomyocyte Isolation Kit (Thermo Fisher). Cells were fixed and permeabilized using a Permeabilization kit for intracellular staining (eBioscience) following the manufacturer's instruction. Cells were then incubated with Cy3-conjugated mouse anti-cardiac Troponin C antibody (Abcam, ab45931, 1:100) and Hoechst 33342 (Invitrogen, 1:1000) at room temperature for 1 h. Cells were washed and percentage of cTnC+ CMs was determined after 20,000 total cell counts by flow cytometry. Percentage of polyploidy CMs was determined by Hoechst 33342 intensity. Flow data was collected using the flow software BD FACS Diva 8.0.3 and analyzed by FlowJo v10.

For analysis of the purity of differentiated hiPSC-CM, dissociated CMs were fixed with 4% PFA and permeabilized using 0.5% saponin. Cells were then incubated with 647-conjugated mouse anti-cardiac TNNT2 antibody (BD Biosciences, clone 13-11, 1:200) for 1 h. Cells were washed and percentage of TNNT2+CM was determined after 10,000 total cell counts by flow cytometry.

Neonate Myocardial Infarction

Neonatal myocardial infarction was performed in P2 pups. Briefly, P2 pups were anaesthetized under isoflurane anesthesia (1-2%). Once pups did not respond to toe pinch, they were moved to a cold platform to undergo hypothermia anesthesia. Each neonate undergoes acute myocardial infarction by ligation of the left anterior descending coronary artery. Thoracic wall incisions were sutured and the wound closed using skin adhesive. Pups were warmed on a warm pad. After confirmation of spontaneous movement pups received a dose of subcutaneous buprenorphine (0.05 mg/kg). Once neonate recovered from hypothermia, they are moved back to its fostering mother's cage.

Compressed Collagen Patches

Compressed acellular collagen patches were prepared as described previously. Briefly, control collagen patches were prepared by adding 1.1 ml DMEM to 0.9 ml of sterile rat tail type I collagen solution in acetic acid (3.84 mg/ml, Millipore) and neutralized with 0.1 M NaOH (˜50 μl). REELIN collagen patches were prepared by adding 20 μg of recombinant human REELIN protein (R&D) into the collagen mix. Then, 0.9 ml of the collagen solution was added into one well of 24-well plates and placed into a tissue culture incubator for 30 min at 37° C. for polymerization. Polymerized collagen gel was then compressed by application of a static compressive stress of ˜1,400 Pa for 5 min as described²⁸. Each collagen patch was then trimmed to 3 even pieces for application in vivo.

Myocardial Infarction and Insertion of Collagen Patches in Adult Mice

Nine-11 week-old NMRI female mice were anaesthetized using an isoflurane inhalational chamber, endotracheally intubated using a 22-gauge angiocatheter and connected to a small animal volume-control ventilator (Harvard Apparatus, Holliston, MA). All mice underwent acute myocardial infarction by ligation of the left anterior descending coronary artery and ligation was considered successful when the LV wall turned pale. Immediately after ligation, prepared collagen patches (with and without REELIN) were sutured (at two points) onto the surface of the ischaemic myocardium (FIG. 4e). The patch size used was ˜ one-third of the 15.6 mm-diameter collagen gel. Animals were kept on a heating pad until they recovered. After confirmation of spontaneous movement, pups received a dose of subcutaneous buprenorphine (0.05 mg/kg).

Echocardiography

Two-dimensional echocardiograms were measured on a 55 MHz probe using Vevo 3100 micro-ultrasound imaging system (VisualSonics), short axis views of the left ventricles were taken at the level of papillary muscles and used to calculate end-diastolic and—systolic dimensions using Vevo LAB 3.2.6 software (VisualSonics). All echocardiography measurements were performed in a blinded manner.

Histology, Immunohistochemistry and Immunofluorescent Staining

For H&E staining, samples were embedded in paraffin and sectioned longitudinally at 6 um thickness and staining was performed according to standard protocols.

For whole mount heart staining, isolated hearts were fixed in 4% PFA o/n and blocked. Antibodies were used as followed: Lyve1 (Goat, R&D, AF2125, 1:200), Endomucin (Rat, Invitrogen, 14-5851-82, 1:500), Reelin (Goat, R&D, AF3820, 1:50), Prox1 (Goat, R&D, AF2727, 1:100) and Cy3-conjugated α-SMA (Mouse, Sigma, C6198, 1:300). Cy3-conjugated donkey anti-goat (Jackson ImmunoResearch, 705-165-147, 1:300) and Cy5-conjugated donkey anti-rat (Jackson ImmunoResearch, 712-175-150, 1:300) were used for immunofluorescent staining.

For cryosections, embryos or isolated hearts were fixed in 4% PFA o/n and dehydrated in 30% sucrose. Samples were embedded in OCT compound and frontal sectioned at 10-um thickness to show four chambers. Primary antibodies were used as follows: α-Actinin (Mouse, Sigma, A7811, 1:500), cardiac Troponin C (Mouse, Abcam ab8295, 1:1000), Ki67 (Rabbit, Invitrogen, SP6, MA5-14520, 1:200), active Caspase-3 (Rabbit, BD Pharmingen, C92-605, 559565, 1:200), Lyve1 (Goat, R&D, AF2125, 1:200), Reelin (Goat, R&D, AF3820, 1:50), Lyve1 (Rabbit, AngioBio, 11-034, 1:500), Prox1 (Rabbit, AngioBio, 11002, 1:500), Prox1 (Goat, R&D, AF2727, 1:100) and Mef2c (Rabbit, LSBio, LSC356188, 1:1000). Secondary antibodies were used as follows: Alexa 488-conjugated donkey anti-rabbit (Invitrogen, A21206, 1:300); Alexa 488-conjugated donkey anti-goat (Invitrogen, A11055, 1:300); Cy3-conjugated donkey anti-rabbit (Jackson ImmunoResearch, 711-165-152, 1:300); Alexa 488-conjugated donkey antimouse (Invitrogen, A21202, 1:300); Cy3-conjugated donkey anti-goat (Jackson ImmunoResearch, 705-165-147, 1:300) and Cy5-conjugated donkey anti-goat (Jackson ImmunoResearch, 705-495-147, 1:300).

For cell staining, cells were fixed in 4% PFA for 30 min on ice, blocked, and incubated with primary antibody against α-Actinin (Mouse, Sigma, A7811, 1:500), cardiac Troponin C (Mouse, Abcam, ab8295, 1:1000), Ki67 (Rabbit, Invitrogen, SP6, MA5-14520, 1:200), Prox1 (Goat, R&D, AF2727, 1:100) and active Caspase-3 (Rabbit, BD Pharmingen, C92-605, 559565, 1:200). Secondary antibodies were used as follows: Alexa 488-conjugated donkey anti-mouse (Invitrogen, A21202, 1:300) and Cy3-conjugated donkey anti-rabbit (Jackson ImmunoResearch, 711-165-152, 1:300). At least 3 heart samples per genotype were used for whole mount staining and 3 sections per heart per staining for immunohistochemistry and immunofluorescent staining, respectively. Cell staining was repeated at least 3 times.

For Masson's Trichrome staining, mouse hearts were harvested and fixed in 4% PFA and embedded. Paraffin sections were cut from apex to base into serial sections at 0.8 μm thickness. Masson's trichrome staining was performed according to standard procedures (Sigma) and used for detection of fibrosis. Scar size was quantified using NIH ImageJ 1.51 software and the percentage of the fibrosis area was calculated relative to left ventricle area.

EdU Administration

To examine the EdU incorporation in Prox1^{ΔLEC/ΔLEC}, Vegfr3^{kd/kd}, β1^{ΔCM/+}; Reln^{+/-} or Prox1^{ΔLEC/ΔLEC} strains, 5-ethynyl-2′-deoxyuridine (EdU, 3 mg/mouse) was administered into pregnant females by intraperitoneal injections. 2 h after injections, mice were euthanized and hearts, livers and kidney collected and cryosectioned as described above. To examine EdU incorporation in control or REELIN patched treated mice after MI, EdU (3 mg/mouse) was injected intraperitoneally for 3 days starting 4 days after MI. Hearts were collected at day 7 and subjected to EdU immunohistochemistry using Click-iT® EdU Alexa Fluor® 488 Imaging Kit (Life Technologies) according to the manufacture's instruction.

Quantitative Reverse Transcription PCR (qRT-PCR)

Total RNAs was extracted using RNeasy Mini Kit (Qiagen). cDNA was generated (Clontech Laboratories) and 20 ng used for qRT-PCR using Power SYBR Green PCR Master Mix (Life Technologies) on a StepOnePlus Real-Time PCR system (Applied Biosystems). At least three individual samples per group were performed for each run of qPCR. Primer sequences used in this study are listed below.

For mouse qPCR:
Bcl2l11:
(SEQ ID NO: 3)
GAGATACGGATTGCACAGGA,
(SEQ ID NO: 4)
ATTTGAGGGTGGTCTTCAGC;
P21
(SEQ ID NO: 5)
GAAAGAAGCGGAAGATCCTCC,
(SEQ ID NO: 6)
GGGCCTCAGGGATTGTTTGG;
Pdcd4
(SEQ ID NO: 7)
GAAATTGGATTTCCGCATCT,
(SEQ ID NO: 8)
TAACCGCTTCACTTCCATT;
Stat1:
(SEQ ID NO: 9)
AGGGGCCATCACATTCACAT,
(SEQ ID NO: 10)
AGATACTTCAGGGGATTCTC;
Trp53ip1:
(SEQ ID NO: 11)
TCCTCAGCAGAGCACACTTC,
(SEQ ID NO: 12)
TCCATTGGACAGGACTCAAA;
Cdc6:
(SEQ ID NO: 13)
AGGGTGACTTTGAGCCAAGA,
(SEQ ID NO: 14)
ATGAAGATTCTGGGGGCTCT;
E2f1:
(SEQ ID NO: 15)
TGCAGAAACGGCGCATCTAT,
(SEQ ID NO: 16)
CCGCTTACCAATCCCCACC;
Pcna:
(SEQ ID NO: 17)
TTGCACGTATATGCCGAGACC,
(SEQ ID NO: 18)
GGTGAACAGGCTCATTCA TCTCT;
Mcm5:
(SEQ ID NO: 19)
GGAGGCTATTGTGCGCATTG,
(SEQ ID NO: 20)
CTGGTCCTCCTGGGTAGTGA;
Ccne2:
(SEQ ID NO: 21)
TCTGTGCATTCTAGCCATCG,
(SEQ ID NO: 22)
ACAAAAGGCACCATCCAGTC;
Reelin:
(SEQ ID NO: 23)
GGACTAAGAATGCTTATTTCC,
(SEQ ID NO: 24)
GGAAGTAGAATTCATCCATCAG;
Rlp32:
(SEQ ID NO: 25)
GCCTCTGGTGAAGCCCAAG,
(SEQ ID NO: 26)
TTGTTGCTCCCATAACCGATGT,
For human qPCR:
(SEQ ID NO: 27)
Reelin: CAATCTGAATGGCGAAACC,
(SEQ ID NO: 28)
CTTTCGCTATAAATCGGAGAGAGA;
Gapdh:
(SEQ ID NO: 29)
TGACCACAGTCCATGCCATC,
(SEQ ID NO: 30)
GACGGACACATTGGGGGTAG;
MMRN1:
(SEQ ID NO: 31)
TTGGATTGGAGGTGCTGTC,
(SEQ ID NO: 32)
GCCTGGTTGGTGTGTATCA;
THBS1:
(SEQ ID NO: 33)
CACCAACCGCATTCCAGAG,
(SEQ ID NO: 34)
TCAGGGATGCCAGAAGGAG;
HSPG2:
(SEQ ID NO: 35)
CTCCATCGTCATCTCCGTCT,
(SEQ ID NO: 36)
GTCTGCCCTTCTGCCACTC;
FN1:
(SEQ ID NO: 37)
CCATCGCAAACCGCTGCCAT;
(SEQ ID NO: 38)
AACACTTCTCAGCTATGGGCTT;
FSTL1:
(SEQ ID NO: 39)
CGATGGACACTGCAAAGAGA,
(SEQ ID NO: 40)
CCAGCCATCTGGAATGATCT;
LAMA4:
(SEQ ID NO: 41)
GCGGCCGAGAAATGCA,
(SEQ ID NO: 42)
AGTCGCAGGGCACACATTC;
SERPINE1:
(SEQ ID NO: 43)
ACAAGTTCAACTATACTGAGTTCACCACGCCC,
(SEQ ID NO: 44)
TGAAACTGTCTGAACATGTCGGTCATTCCC.

All sequences are included forward and reversed and are annotated from 5′ to 3.

RNAseq

Total RNA was extracted using the RNeasy Mini Kit (Qiagen) according to the manufacturer's instructions. Extracted total RNAs were quantitated by NanoDrop and RNA integrity number value measured with an Agilent Bioanalyzer. In all RNAseq samples, quality control was performed using the 2100 Bioanalyzer (Agilent). RNA library was prepared using the TruSeq mRNA-Seq Library Prep and sequenced using the HiSEQ Next-generation Sequencing System at the NUSeq Core.

Imaging Acquisition and Quantification

Confocal images in FIG. 1a, d-i, FIGS. 5a, 7b, 8b, 10i and 12g were acquired using a Nikon W1 Dual CAM Spinning Disk confocal microscope. All other confocal images were acquired using Zeiss LSM510 laser-scanning confocal microscope. All confocal images represent maximum projection images of z stacks. For quantifications of CM proliferation and apoptosis, images were taken from 3 myocardium regions of frontal heart sections: myocardium nearby left ventricle, myocardium nearby right ventricle and septum. At least 9 images were taken from each heart (at least 3 images/region) and at least 3 hearts from each genotype were quantified. Bright field images were taken using a Leica stereomicroscope. Embryo body length was measured from head to tail (crown-rump) using Image J 1.51 with all the images under the same magnification. CM cell size, as well as fibrosis area were also measured by Image J 1.51 software with all the images under the same magnification.

Statistical Analysis

No statistical analysis was used to predetermine sample size. Statistical analysis was performed using GraphPad Prism 7 and Microsoft Excel 2016. Differences between two groups were determined by two-tailed unpaired t-test, and differences between multiple groups were calculated using one-way ANOVA or two-way ANOVA. Differences with p<0.05 (*), p<0.01 (**), and p<0.001 (***) were considered statistically significant.

RNAseq raw data have been deposited to the Gene Expression Omnibus (GEO) repository with accession number GSE158504.

### REFERENCES

1. Oliver, G., Kipnis, J., Randolph, G. J. & Harvey, N. L. The Lymphatic Vasculature in the 21st Century: Novel Functional Roles in Homeostasis and Disease. Cell 182, 270-296 (2020).
2. Klotz, L. et al. Cardiac lymphatics are heterogeneous in origin and respond to injury. Nature 522, 62-7 (2015).
3. Vuorio, T., Tirronen, A. & Yla-Herttuala, S. Cardiac Lymphatics—A New Avenue for Therapeutics? Trends in Endocrinology and Metabolism 28, 285-296 (2017).
4. Henri, O. et al. Selective Stimulation of Cardiac Lymphangiogenesis Reduces Myocardial Edema and Fibrosis Leading to Improved Cardiac Function Following Myocardial Infarction. Circulation 133, 1484-97; discussion 1497 (2016).
5. Vieira, J. M. et al. The cardiac lymphatic system stimulates resolution of inflammation following myocardial infarction. J. Clin. Invest 128, 3402-3412 (2018).
6. Harrison, M. R. et al. Late developing cardiac lymphatic vasculature supports adult zebrafish heart function and regeneration. Elife 8, pii: e42762 (2019).
7. Harvey, N. L. et al. Lymphatic vascular defects promoted by Prox1 haploinsufficiency cause adult-onset obesity. Nat. Genet. 37, 1072-1081 (2005).
8. Wigle, J. T. & Oliver, G. Prox1 function is required for the development of the murine lymphatic system. Cell 98, 769-778 (1999).
9. Johnson, N. C. et al. Lymphatic endothelial cell identity is reversible and its maintenance requires Prox1 activity. Genes Dev 22, 3282-3291 (2008).
10. Sorensen, I., Adams, R. H. & Gossler, A. DLL1-mediated Notch activation regulates endothelial identity in mouse fetal arteries. Blood 113, 5680-8 (2009).
11. Zhang, L. et al. VEGFR-3 ligand-binding and kinase activity are required for lymphangiogenesis but not for angiogenesis. Cell Res. 20, 1319-1331 (2010).
12. Ross, O. N., Moore, C. M., Suggett, D. J., Macintyre, H. L. & Geider, R. J. Endothelial-Cardiomyocyte Interactions in Cardiac Development and Repair. Annu. Rev Physiol 68:1-736, 1835-1852 (2006).
13. Brutsaert, D. L. & Cotran, R. S. Cardiac endothelial-myocardial signaling: its role in cardiac growth, contractile performance, and rhythmicity. Physiol. Rev. 83, 59-115 (2003).
14. Jossin, Y. Neuronal Migration and the Role of Reelin During Early Development of the Cerebral Cortex. Mol. Neurobiol. 30, 225-252 (2004).
15. D'Arcangelo, G. Reelin in the Years: Controlling Neuronal Migration and Maturation in the Mammalian Brain. Adv. Neurosci. 2014, 1-19 (2014).
16. Lutter, S., Xie, S., Tatin, F. & Makinen, T. Smooth muscle-endothelial cell communication activates Reelin signaling and regulates lymphatic vessel formation. J. Cell Biol. 197, 837-849 (2012).
17. D'Arcangelo, G. et al. A protein related to extracellular matrix proteins deleted in the mouse mutant reeler. Nature 374, 719-723 (1995).
18. Lane-Donovan, C. et al. Reelin protects against amyloid R toxicity in vivo. Sci. Signal. 8, ra67-ra67 (2015).
19. Srinivasan, R. S. et al. Lineage tracing demonstrates the venous origin of the mammalian lymphatic vasculature. Genes and Development 21, 2422-2432 (2007).
20. Trommsdorff, M. et al. Reeler/Disabled-like disruption of neuronal migration in knockout mice lacking the VLDL receptor and ApoE receptor 2. Cell 97, 689-701 (1999).
21. Hiesberger, T. et al. Direct binding of Reelin to VLDL receptor and ApoE receptor 2 induces tyrosine phosphorylation of disabled-1 and modulates tau phosphorylation. Neuron 24, 481-9 (1999).
22. Lin, L. et al. Reelin promotes the adhesion and drug resistance of multiple myeloma cells via integrin β1 signaling and STAT3 Oncotarget 7, (2016).
23. Dulabon, L. et al. Reelin binds alpha3beta1 integrin and inhibits neuronal migration. Neuron 27, 33-44 (2000).
24. Beffert, U. et al. Reelin-mediated Signaling Locally Regulates Protein Kinase B/Akt and Glycogen Synthase Kinase 30. J. Biol. Chem. 277, 49958-49964 (2002).
25. Jossin, Y. & Goffinet, A. M. Reelin signals through phosphatidylinositol 3-kinase and Akt to control cortical development and through mTor to regulate dendritic growth. Mol. Cell. Biol. 27, 7113-24 (2007).
26. Ieda, M. et al. Cardiac fibroblasts regulate myocardial proliferation through beta1 integrin signaling. Dev. Cell 16, 233-44 (2009).
27. Porrello, E. R. et al. Transient Regenerative Potential of the Neonatal Mouse Heart. Science 331, 1078-1080 (2011).
28. Wei, K. et al. Epicardial FSTL1 reconstitution regenerates the adult mammalian heart. Nature 525, 479-485 (2015).
29. Serpooshan, V. et al. The effect of bioengineered acellular collagen patch on cardiac remodeling and ventricular function post myocardial infarction. Biomaterials 34, 9048-9055 (2013).
30. Karkkainen, M. J. et al. A model for gene therapy of human hereditary lymphedema. Proc. Natl. Acad. Sci 98, 12677-82. (2001).
31. Burridge, P. W. et al. Chemically defined generation of human cardiomyocytes. Nat. Methods 11, 855-860 (2014).
32. Burridge, P. W., Holmström, A. & Wu, J. C. Chemically Defined Culture and Cardiomyocyte Differentiation of Human Pluripotent Stem Cells. in Current Protocols in Human Genetics 87, 21.3.1-21.3.15 (John Wiley & Sons, Inc., 2015).
33. Liu, X. et al. Rasip1 controls lymphatic vessel lumen maintenance by regulating endothelial cell junctions. Development 145, dev165092 (2018).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating a disease or condition of the heart in a subject in need thereof, the method comprising: administering to the subject a composition comprising a reelin polypeptide or a variant thereof.

2. The method of claim 1 wherein the disease or conditions of the heart comprises one or more of: (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) and endocarditis (bacterial, viral, or fungal).

3. The method of claim 2, wherein the disease or condition comprise myocardial infarction.

4. The method of claim 1, wherein treatment provides an alleviation of at least one symptom of the cardiac disease or condition sooner than an untreated subject with the same or similar disease or condition.

5. The method of claim 4, wherein the at least one symptom comprises one or more of: (1) arrhythmia; (2) tachycardia; (3) bradycardia; (4) chest pain (angina); (5) fainting; (6) swollen feet or ankles; (7) cyanosis; (8) dizziness; (9) decreased cardiac lymphatics; (10) cardiac fluid accumulation and/or cardiac inflammation; (11) decreased systolic function; (12) atherosclerotic plaque formation; (13) delayed cardiac healing process and cardiac repair; (14) adverse cardiac remodeling; (15) shortness of breath; (16) weakness or fatigue.

6. A method for improving cardiac function in a subject in need thereof, the method comprising: administering an effective amount of reelin polypeptide, or a variant thereof.

7. The method of claim 6, wherein the subject has been diagnosed with a cardiac disease or condition or has suffered a cardiac injury.

8. The method of claim 7, wherein the cardiac disease or cardiac injury comprises a myocardial infarction.

9. The method of claim 8, wherein improved cardiac function includes a measurable improvement in on or more of: (1) arrhythmia; (2) tachycardia; (3) bradycardia; (4) chest pain (angina); (5) fainting; (6) swollen feet or ankles; (7) cyanosis; (8) dizziness; (9) decreased cardiac lymphatics; (10) cardiac fluid accumulation and/or cardiac inflammation; (11) systolic function; (12) atherosclerotic plaque formation; (13) cardiac healing process and cardiac repair; (14) cardiac remodeling; (15) shortness of breath; (16) weakness or fatigue.

10. A method to stimulate the formation of new lymphatics in cardiac tissue in a subject in need thereof, the method comprising administering an effective amount of reelin polypeptide, or a variant thereof.

11. The method of claim 10, wherein the subject in need thereof has suffered or is at risk of a cardiac disease or conditions selected from the group consisting of: (1) coronary artery disease; (2) arrhythmia; (3) bradycardia; (4) tachycardia; (5) congenital heart disease or defect; (6) myocardial infarction (heart attack); (7) cardiomyopathy; (8) heart valve disease; (9) pericardial disease; (10) rheumatic heart disease; (11) stroke; (12) heart failure; (13) ischemia/reperfusion injury; (14) trauma; (15) cardiac inflammation; (16) cardiac edema; (17) and endocarditis (bacterial, viral, or fungal).

12. The method of claim 10, wherein the subject has suffered or is at risk of myocardial infarction.

13. The method of claim 1, wherein the reelin polypeptide comprises SEQ ID NO: 1 or a fragment thereof.

14. The method of claim 1, wherein administration comprises providing the reelin polypeptide to the subject in a collagen patch.

15. The method of claim 1, wherein the reelin polypeptide is recombinant.

16. A composition comprising: a reelin polypeptide embedded in a collagen patch.

17. The composition of claim 16, wherein the reelin polypeptide comprises SEQ ID NO: 1.

18. The method of claim 6, wherein administration comprises providing the reelin polypeptide to the subject in a collagen patch.

19. The method of claim 10, wherein administration comprises providing the reelin polypeptide to the subject in a collagen patch.

20. The method of claim 19, wherein the subject has suffered or is at risk of myocardial infarction.
